(19) United States
(12) Patent Application Publication
(10) Pub. No.: US 2024/0352083 A1
(43) Pub. Date: Oct. 24, 2024

(54) ANTIBODIES COMPRISING A POLYPEPTIDE INSERTED IN FRAMEWORK 3 REGION

(71) Applicant: UCB Biopharma SRL, Brussels (BE)

(72) Inventors: Ralph Adams, Slough (GB); Terence Seward Baker, Slough (GB); Xiaofeng Liu, Slough (GB)

(21) Appl. No.: 18/647,911

(22) Filed: Apr. 26, 2024

Related U.S. Application Data
(63) Continuation of application No. 17/259,090, filed on Jan. 8, 2021, now Pat. No. 11,999,772.

(30) Foreign Application Priority Data
Jul. 11, 2018 (GB) 1811368.8

(51) Int. Cl. C07K 14/54; C07K 14/505; C07K 14/535; C07K 14/55; C07K 16/18; C07K 16/24; C07K 16/28

(52) U.S. Cl. CPC C07K 14/5443; C07K 14/505; C07K 14/535; C07K 14/5428; C07K 14/55; C07K 16/18; C07K 16/244; C07K 16/248; C07K 16/2809; C07K 16/2818; C07K 2317/24; C07K 2317/31; C07K 2317/55; C07K 2317/56; C07K 2317/567; C07K 2317/569; C07K 2317/622; C07K 2317/76

(57) ABSTRACT

The present invention provides antibodies comprising a variable (V) domain and an insert polypeptide, wherein the insert polypeptide is within the framework 3 region of the V domain.

### CROSS-REFERENCE TO RELATED APPLICATION

This application is a continuation of U.S. patent application Ser. No. 17/259,090, filed Jan. 8, 2021, which in turn is a U.S. 35 U.S.C. § 371 national stage under 35 U.S.C. § 371 of International Patent Application No. PCT/EP2019/068570 filed Jul. 10, 2019, which claims the benefit of and priority to Great Britain Application No. 1811368.8, filed on Jul. 11, 2018, the content of each of which is incorporated by reference herein in its entirety and for all purposes.

### INCORPORATION BY REFERENCE OF MATERIAL SUBMITTED ELECTRONICALLY

The application is submitted with a sequence listing in electronic format. The sequence listing is provided as a file entitled “26300A_SubSeqListing.xml,” created Jun. 6, 2024, which is 203,222 bytes in size. The information in the sequence listing is incorporated herein by reference in its entirety.

### FIELD OF THE INVENTION

The present invention relates to antibodies comprising a variable (V) domain and an insert polypeptide within the framework 3 region of the V domain. Such antibodies may provide improved functionality and/or pharmacokinetics for the insert polypeptide.

### BACKGROUND OF THE INVENTION

The high specificity and affinity of antibodies makes them ideal diagnostic and therapeutic agents. Advances in the field of recombinant antibody technology have resulted in the production of antibody fragments, such as Fv, Fab, Fab′ and F(ab′)₂ fragments. These smaller molecules retain the antigen binding activity of whole antibodies and can also exhibit improved tissue penetration and pharmacokinetic properties in comparison to whole immunoglobulin molecules. Indeed, antibody fragments are proving to be versatile therapeutic agents. Whilst such fragments appear to exhibit a number of advantages over whole immunoglobulins, they also suffer from an increased rate of clearance from serum since they lack the Fc domain that imparts a long lifetime in vivo (Medasan et al., 1997, J. Immunol. 158:2211-2217). Previous approaches to improving the serum half-life of antibody molecules include conjugation to agents or domains which interact with the FcRn receptor, such as human serum albumin (HSA). PEG has also been shown to improve serum half-life.

Antibodies with dual specificity, i.e. which bind to two different antigens have been previously described (for reviews, see Segal et al., 1999, Curr. Opin. Immunol. 11:558-562; Pluckthun & Pack, 1997, Immunotechnology, 3:83-105; Fischer and Leger, 2007, Pathobiology, 74: 3-14). Previous approaches to making hetero-bispecific antibody-based molecules have generally employed chemical cross-linking or protein engineering techniques. Chemical cross-linking suffers from poor yields of hetero- and homo-dimer formation, and the requirement for their subsequent chromatographic separation. Protein engineering approaches have either been highly elaborate (e.g. knobs-into-holes engineering; Ridgway et al., 1996, Protein Eng. 9(7):617-621) or have used molecules with inappropriate stability characteristics (e.g. diabodies, scFv). In some cases, bispecific antibodies can also suffer from steric hindrance problems such that both antigens cannot bind simultaneously to each antibody arm. Single variable domain antibodies also known as single domain antibodies or sdAbs, correspond to the variable regions of either the heavy (VH) or light (VL) chain of an antibody. Murine single-domain antibodies were described by Ward et al., 1989, Nature, 341, 544-546. Human and ‘camelised’ human single domain antibodies have also been described (Holt et al., 2003, Trends in Biotechnology, 21, 484-490). Single domain antibodies have also been obtained from the camelids (camels and llamas) and cartilaginous fish (wobbegong and nurse sharks). These organisms have evolved high affinity single V-like domains (called VHH in camelids and V-NAR in sharks), mounted on an Fc-equivalent constant domain framework as an integral and crucial component of their immune system (see Holliger & Hudson, for a review; 2005, Nature Biotechnology, 23(9):1126-1136).

As such, there remains a need to provide further engineered antibodies such as fusion proteins comprising an antibody portion which exhibits one or more desired properties, such as increased functionality (e.g. multi-valent binding) or improved pharmacokinetics (e.g. increased half-life in serum). It is the object of the present invention to provide a novel engineered antibody format which exhibit increased functionality (e.g. increased binding to its antigen, bi-specific binding, or multi-valent binding) or improved pharmacokinetics (e.g. increased half-life in serum). In particular, the present invention provides a novel bispecific antibody format, in particular stable and capable of simultaneously binding two antigens.

### SUMMARY OF THE INVENTION

In one aspect, the present invention provides an antibody comprising a variable (V) domain and an insert polypeptide, wherein the insert polypeptide is within the framework 3 region of the V domain.

In some embodiments, the V domain is a VH domain, and the antibody may further comprise a VL domain.

In some embodiments, the V domain is a VL domain, and the antibody may further comprise a VH domain.

In some embodiments, the insert polypeptide is between amino acid residues 73 and 76 of the VH domain. For example, the insert polypeptide may be in the VH domain: (i) between amino acid residues 73 and 74; (ii) between amino acid residues 74 and 75; or (iii) between amino acid residues 75 and 76.

In some embodiments, one or more amino acid residues between 73 and 76 of the VH domain are replaced by the insert polypeptide.

In some embodiments, the insert polypeptide is between amino acid residues 67 and 70 of the VL domain. For example, the insert polypeptide may be in the VL domain: (i) between amino acid residues 67 and 68; (ii) between amino acid residues 68 and 69; or (iii) between amino acid residues 69 and 70.

In some embodiments, one or more amino acid residues between 67 and 70 of the VL domain are replaced by the insert polypeptide.

In some embodiments, the antibody of the invention further comprises a linker sequence, preferably of at least one, two, three, four, five, six, seven, eight, nine or ten amino acids joining the N- and/or C-terminal end of the insert polypeptide to the framework 3 region.

In some embodiments, the antibody of the invention is a full-length antibody or a binding fragment thereof. In some embodiments, the antibody is a full-length IgG or a binding fragment thereof. In some embodiments, the antibody is a Fab, Fab′, F(ab′)₂, VHH, or scFv, in particular dsscFv.

In some embodiments, the antibody of the invention is humanised.

In some embodiments, the antibody of the invention comprises a V domain which binds to human serum albumin (HSA). In one embodiment, the antibody comprises a VL domain comprising CDR-L1 having the sequence SEQ ID NO: 3; CDR-L2 having the sequence SEQ ID NO: 4; CDR-L3 having the sequence SEQ ID NO: 5. In one embodiment, the antibody comprises a VL domain comprising SEQ ID NO: 2. In one embodiment, the antibody comprises a light (L) chain comprising SEQ ID NO: 1. In one embodiment, the antibody comprises a VH domain comprising CDR-H1 having the sequence SEQ ID NO: 8; CDR-H2 having the sequence SEQ ID NO: 9; CDR-H3 having the sequence SEQ ID NO: 10. In one embodiment, the antibody comprises a VH domain comprising SEQ ID NO: 7. In one embodiment, the antibody comprises a heavy (H) chain comprising SEQ ID NO: 6.

In some embodiments, the insert polypeptide is a cytokine, preferably wherein the cytokine is selected from IL-10, IL-15, IL-2, G-CSF, GM-CSF and EPO. In other embodiments, the insert polypeptide is sclerostin. In other embodiments, the insert polypeptide is a second antibody, preferably wherein the second antibody is an scFv, in particular a dsscFv, or a domain antibody such as a VH or VL or VHH. The VHH may comprise a sequence selected from SEQ ID NOs: 67, 69 and 89. In such embodiments, the antibody may bind its cognate antigen by said V domain with a binding affinity which is at least 50%, 60%, 70%, 80% or 90% when compared to the antibody without the insert polypeptide present.

In some embodiments, the second antibody binds to the same antigen as the V domain. Preferably, the second antibody binds to a different epitope as the V domain. In such embodiments, the antibody may bind its cognate antigen with a binding affinity which is greater than when compared to the antibody without the second antibody present. In some embodiments, the second antibody binds to a different antigen to the first antibody, i-e the antibody comprising a V domain. In some embodiments, the second antibody binds to IL-17 and the first antibody, i-e the antibody comprising a V domain, binds to HSA. In some embodiments, the second antibody binds to CD3 and the first antibody, i-e the antibody comprising a V domain, binds to CD28. In some embodiments, the second antibody binds to CD28 and the first antibody, i-e the antibody comprising a V domain binds to CD3.

The present invention also provides isolated polynucleotide(s), such as a DNA molecule(s), and cloning or expression vector(s) which encodes an antibody disclosed herein. Also provided is a host cell comprising said polynucleotide(s) or vector(s). The present invention further provides a process for the production of an antibody as disclosed herein, comprising culturing said host cell and isolating the antibody.

In another aspect, the present invention also provides a pharmaceutical composition comprising an antibody comprising a V domain and an insert polypeptide as disclosed herein in combination with one or more of a pharmaceutically acceptable excipient, diluent or carrier.

Also provided is said antibody or said pharmaceutical composition for use in therapy. Also provided is a method of therapy comprising administering said antibody or said pharmaceutical composition to a subject in need of the therapy. Also provided is the use of said antibody for the manufacture of a medicament.

### BRIEF DESCRIPTION OF THE DRAWINGS

FIG. 1: (A) Typical Fab structure, highlighting framework 3 loop region; (B) highlighting certain residues in the heavy chain variable domain framework 3 region; (C) highlighting certain residues in the light chain variable domain framework 3 region.

FIG. 2: (A) SPR sensorgram showing CA645/IL-15_{Fwk3} binding to IL-15 receptor chains; (B) Modelled structure showing the IL-15 insert into framework 3 of the CA645 VH domain.

FIG. 3: (A) SPR sensorgram showing CA645/IL-2_{Fwk3} binding to IL-2 receptor 3 chain with and without added IL-15; (B) Modelled structure showing the IL-2 insert into framework 3 of the CA645 VH domain.

FIG. 4: (A) SPR sensorgram showing CA645/G-CSF_{Fwk3} binding to HSA and/or G-CSF receptor; (B) Modelled structure showing the G-CSF insert into framework 3 of the CA645 VH domain.

FIG. 5: (A) SPR sensorgram showing CA645/GM-CSF_{Fwk3} (human) binding to GM-CSF receptor, with comparison to a CA645/GM-CSF_{CH1} (human) construct; (B) Modelled structure showing the GM-CSF insert into framework 3 of the CA645 VH domain.

FIG. 6: SPR sensorgram showing CA645/GM-CSF_{Fwk3} (murine) binding to GM-CSF receptor, with comparison to a CA645/GM-CSF_{CH1} (murine) construct.

FIG. 7: (A) SPR sensorgram showing CA645/EPO_{Fwk3} binding to EPO receptor with and without added EPO; (B) Modelled structure showing the EPO insert into framework 3 of the CA645 VH domain.

FIG. 8: (A) SPR sensorgram showing CA645/VHH6_{Fwk3} binding to IL-6, HSA, IL-6:gp80 with or without HSA; (B) Modelled structure showing the VHH6 insert into framework 3 of the CA645 VH domain.

FIG. 9: (A) SPR sensorgram showing CA645/VHH15_{Fwk3} binding to HSA and/or IL-6; (B) Modelled structure showing the VHH15 insert into framework 3 of the CA645 VH domain.

FIG. 10: (A) SPR sensorgram showing CA645/Sclerostin_{Fwk3} (core or full-length) binding to LRP6 extracellular domain with various linker constructs; (B) Modelled structure showing the sclerostin core insert into framework 3 of the CA645 VH domain.

FIG. 11: SPR sensorgram showing CA645/Sclerostin_{Fwk3} (core or full-length) binding to LRP4 extracellular domain with various linker constructs.

FIG. 12: (A) SPR sensorgram showing CA645/(IL-10_{Fwk3})₂ having different linker sequences binding to IL-10 receptor; (B) Modelled structure showing the IL-10 dimer insert into framework 3 of the CA645 VH domain.

FIG. 13: SPR sensorgram showing CA497/IL-15_{Fwk3} binding to IL-2 receptor R chain with and without added IL-15.

FIG. 14: SPR sensorgram showing CA240/IL-15_{Fwk3} binding to IL-2 receptor R chain with and without added IL-15.

FIG. 15: SPR sensorgram showing dilution series for HyHEL5/VHH D3L11_{Fwk3} binding to lysozyme.

FIG. 16: SPR sensorgram showing dilution series for HyHEL5/VHH15_{Fwk3} binding to lysozyme.

FIG. 17: SPR sensorgram showing dilution series for CA645/VHH D3L11_{Fwk3} binding to lysozyme.

FIG. 18: Representation of potential binding schematic of HyHEL5/VHH D3L11_{Fwk3} to lysozyme.

FIG. 19: SPR sensorgram showing CA645/IL-15_{Fwk3} having different linker sequences binding to IL-15 receptor chain.

FIG. 20: DSC curves (solid line: CA645 Fab; dotted line: CA497 dsscFv; inverted triangle: CA645 Fab-Fwk3 CA497 dsscFv (vLvH); triangle: CA645 Fab-Fwk3 CA497 dsscFv (vHvL)).

FIG. 21: Inhibition curves of IL-17A in absence (solid circle) or in presence (open circle) of HSA; (A) CA497 IgG4; (B) CA645 Fab-Fwk3 CA497 dsscFv (vHvL); (C) CA645 Fab-Fwk3 CA497 dsscFv (vLvH).

FIG. 22: SPR analysis showing the binding of IL-17A and HSA to immobilized CA645 IgG4P comprising an anti-IL-17 CA497 dsscFv (vHvL) inserted into Fwk3 region.

FIG. 23: DSC curves. (A) IgG4P anti-CD28 comprising a dsscFv anti-CD3 within the Fwk3 region (solid line); IgG4P anti-CD3 comprising a dsscFv anti-CD3 within the Fwk3 region (dotted line). (B) Fab anti-CD28 comprising a dsscFv anti-CD3 within the Fwk3 region (solid line); Fab anti-CD3 comprising a dsscFv anti-CD3 within the Fwk3 region (dotted line).

FIG. 24: T cell activation assay in PBMC. Open triangle: anti-CD28 Fab Fwk3-anti-CD3 dsscFv; solid circle: anti-CD3 Fab; solid inverted triangle: anti-CD28 Fab; solid circle: anti-CD3 Fab Fwk3-anti-CD3 dsscFv; open square: anti-CD28 IgG4P Fwk3-anti-CD3 dsscFv; solid triangle: anti-CD3 IgG4P-Fwk3 anti-CD3 dsscFv; * symbol: OKT3; open circle: Cells only control. (A) CD4 T cell activation (patient 1) (B) CD4 T cell activation (patient 2) (C) CD8 T cell activation (patient 1) (D) CD8 T cell activation (patient 2).

### DETAILED DESCRIPTION

### Antibody

The present invention provides an antibody comprising a variable (V) domain and an insert polypeptide, wherein the insert polypeptide is within the framework 3 (Fwk3) region of the V domain.

The antibody of the present invention is typically constructed by incorporating the insert polypeptide into the Fwk3 region of a V domain of an antibody. The V domain may for example be a VH domain or a VL domain. The antibody into which the insert polypeptide is incorporated may be considered a scaffold antibody and/or where the insert polypeptide is also an antibody, the antibody into which the insert polypeptide is incorporated may alternatively be termed the first antibody while the insert polypeptide is termed the second antibody. The terms “scaffold antibody” and “first antibody” can be used interchangeably in the context of the present disclosure to refer to the antibody into which the insert polypeptide is incorporated.

In some embodiments, the antibody of the invention is a full-length antibody or a binding fragment thereof. In some embodiments, the antibody is a full-length IgG or a binding fragment thereof. In some embodiments, the antibody is a full-length IgG1. In some embodiments, the antibody is a full-length IgG4. In some embodiments, the antibody is a full-length IgG4P, wherein the serine at position 241 (numbered according to the Kabat numbering system) has been changed to proline. In some embodiments, the antibody is a Fab, Fab′, F(ab′)₂, VHH, or scFv. In some embodiments, the antibody is a disulphide stabilised scFv or “dsscFv”. “Disulphide-stabilised single chain variable fragment” or “dsscFv” as employed herein refer to a single chain variable fragment which is stabilised by a peptide linker between the V_{H} and V_{L} variable domain and also includes an inter-domain disulphide bond between V_{H} and V_{L}. (see for example, Weatherill et al., Protein Engineering, Design & Selection, 25 (321-329), 2012; WO2007109254. In some embodiments, the inter-domain disulphide bond between V_{H} and V_{L} is formed between positions V_{H}44 and V_{L}100 (numbering according to Kabat numbering system).

The term ‘antibody’ as used herein generally relates to full length (intact, whole) antibodies, i.e. comprising elements of two heavy chains and two light chains in the case of an IgG antibody.

The terms “antibody construct”, “engineered antibody”, “antibody fusion protein” can be used interchangeably in the context of the present invention to refer to an antibody of the invention.

Binding fragments of antibodies generally comprise at least one variable light (VL) or variable heavy (VH) domain and include: single chain antibodies (e.g. a full length heavy chain or light chain), Fab, modified Fab, Fab′, modified Fab′, F(ab′)₂, Fv, Fab-Fv, Fab-dsFv, single domain antibodies (sdAb, e.g. VH or VL or VHH), scFv, dsscFv, Bis-scFv, diabodies, tribodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody binding fragments are well known in the art (see for example Verma et al., 1998, Journal of Immunological Methods, 216, 165-181). For example, antibody binding fragments may be obtained from any whole antibody, especially a whole monoclonal antibody, using any suitable enzymatic cleavage and/or digestion techniques, e.g. treatment with pepsin. Alternatively, the antibody starting material may be prepared by the use of recombinant DNA techniques involving the manipulation and re-expression of DNA encoding antibody variable and/or constant regions. Standard molecular biology techniques may be used to modify, add or delete amino acids or domains as desired. Any alterations to the variable or constant regions are still encompassed by the terms ‘variable’ and ‘constant’ regions as used herein. The antibody fragment starting material may be obtained from any species including, for example, mouse, rat, rabbit, hamster, camel, llama, goat or human. Parts of the antibody fragment may be obtained from more than one species; for example, the antibody fragments may be chimeric. In one example, the constant regions are from one species and the variable regions from another. The antibody fragment starting material may also be modified. In another example, the variable region of the antibody fragment has been created using recombinant DNA engineering techniques. Such engineered versions include those created for example from natural antibody variable regions by insertions, deletions or changes in or to the amino acid sequences of the natural antibodies. Particular examples of this type include those engineered variable region domains containing at least one CDR and, optionally, one or more framework amino acids from one antibody and the remainder of the variable region domain from a second antibody. The Fab-Fv format was first disclosed in WO2009/040562, and the disulphide stabilised version thereof, the Fab-dsFv, was first disclosed in WO2010/035012. Other antibody fragments include the Fab and Fab′ fragments described in International patent applications WO2005/003169, WO2005/003170 and WO2005/003171. Multi-valent antibodies may comprise multiple specificities e.g. bispecific or may be monospecific (see for example WO 92/22583 and WO05/113605). One such example of the latter is a Tri-Fab (or TFM) as described in WO92/22583. Another multi-specific format is described in WO2015/197772.

A typical Fab′ molecule comprises a heavy and a light chain pair in which the heavy chain comprises a variable region VH, a constant domain CH1 and a natural or modified hinge region and the light chain comprises a variable region VL and a constant domain CL.

A binding domain of an antibody typically comprises 6 CDRs, three from a heavy chain and three from a light chain. In such an antibody, the CDRs are in a framework and together form a variable region. Thus, in one embodiment the antibody comprises a binding domain specific for an antigen comprising a light chain variable region and a heavy chain variable region. In other embodiments, for example wherein the antibody is a single domain antibody, the binding domain may only comprise the 3 CDRs from a single V domain of either a heavy chain or a light chain. Thus, in one embodiment the antibody comprises a binding domain specific for an antigen comprising one of a light chain variable region (VL domain) or a heavy chain variable region (VH or VHH domain). It will be appreciated that the V domain which binds the antigen may do so alone, or co-operatively with another V domain, for example as a complementary VH/VL pair. Accordingly, the antibody comprising a V domain may bind its antigen co-operatively with a complementary V (VH or VL) domain. In one embodiment, the V domain is a VH domain, and the antibody of the invention further comprises a VL domain, wherein the VH domain binds its antigen co-operatively with the VL domain as a VH/VL pair, i-e the antibody of the invention comprises a VH domain and binds its antigen co-operatively with a complementary VL domain. In one embodiment, the V domain is a VL domain, and the antibody of the invention further comprises a VH domain, wherein the VL binds its antigen co-operatively with the VH domain as a VH/VL pair, i-e the antibody of the invention comprises a VL domain and binds its antigen co-operatively with a complementary VH domain.

The binding domain of the antibody of the invention typically refers to the VH, VL, or pair of VH/VL which binds the antigen.

Antibodies contain generally one or more binding domains. For example, an unmodified Fab fragment comprises one binding domain formed by a VH/VL pair, and a whole IgG comprises two binding domains, each one being formed by a VH/VL pair, wherein the two binding domains are identical (monospecific antibody) or different (bispecific or bivalent antibody). In one example, the antibody of the invention may be a Fab fragment comprising a VH domain and an insert polypeptide within the Fwk3 region of the VH domain, and further comprising a VL domain which forms a complementary pair with the VH domain. In one example, the VL domain may be unmodified (i-e the antibody of the invention is a Fab fragment comprising a single insert polypeptide within the Fwk3 region of the VH domain) or may comprise an insert polypeptide in its Fwk3 region (i-e the antibody of the invention is a Fab fragment comprising two insert polypeptides, identical, similar or different, one inserted within the Fwk3 region of the VH domain and the other one inserted within the Fwk3 region of the VL domain). In one example, the antibody of the invention may be a Fab fragment comprising a VL domain and an insert polypeptide within the Fwk3 region of the VL domain, and further comprising a VH domain which forms a complementary pair with the VL domain. In one example, the VH domain may be unmodified (i-e the antibody of the invention is a Fab fragment comprising a single insert polypeptide within the Fwk3 region of the VL domain) or may comprise an insert polypeptide in its Fwk3 region (i-e the antibody of the invention is a Fab fragment comprising two insert polypeptides, identical, similar or different, one inserted within the Fwk3 region of the VL domain and the other one inserted within the Fwk3 region of the VH domain). In another example, the antibody of the invention is a full IgG comprising a VH domain and an insert polypeptide within the Fwk3 region of the VH domain, and further comprising a VL domain which forms a complementary pair with the VH domain. In one example, the VL domain may be unmodified (i-e the antibody of the invention is an IgG comprising two insert polypeptides, each one being inserted into one of the two VH domains of the IgG) or may comprise an insert polypeptide in its Fwk3 region (i-e the antibody of the invention is an IgG comprising four insert polypeptides, identical, similar or different, each of them being inserted into the Fwk3 region of each one of its two VH and two VL domains). In another example, the antibody of the invention is a full IgG comprising a VL domain and an insert polypeptide within the Fwk3 region of the VL domain, and further comprising a VH domain which forms a complementary pair with the VL. The VH domain may be unmodified (i-e the antibody of the invention is an IgG comprising two insert polypeptides, each one being inserted into one of the two VL domains of the IgG) or may comprise an insert polypeptide in its Fwk3 region (i-e the antibody of the invention is an IgG comprising four insert polypeptides, identical, similar or different, each of them being inserted into the Fwk3 region of each one of its two VL and two VH domains).

The residues in antibody variable domains are conventionally numbered according to a system devised by Kabat et al. This system is set forth in Kabat et al., 1987, in Sequences of Proteins of Immunological Interest, US Department of Health and Human Services, NIH, USA. This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence by methods known in the art.

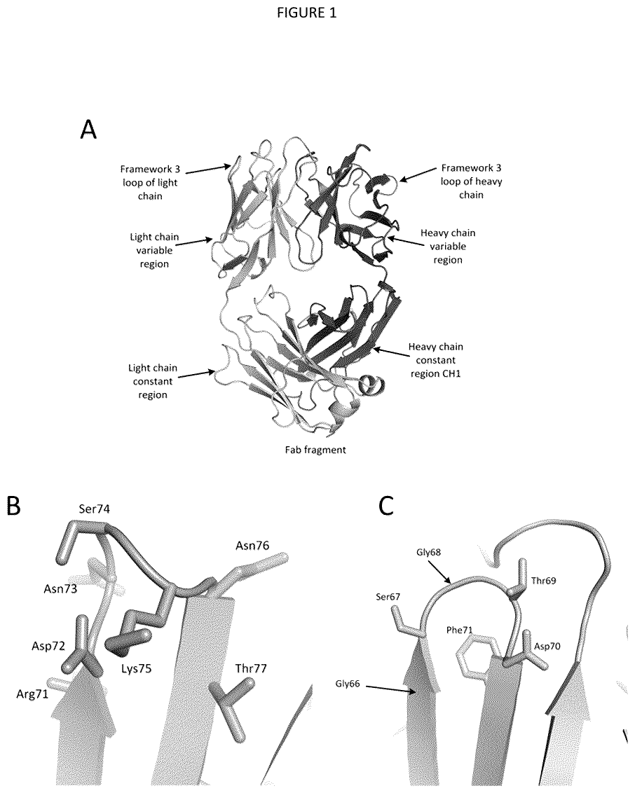

The CDRs of the heavy chain variable domain are located at residues 31-35 (CDR-H1), residues 50-65 (CDR-H2) and residues 95-102 (CDR-H3) according to the Kabat numbering system. The CDRs of the light chain variable domain are located at residues 24-34 (CDR-L1), residues 50-56 (CDR-L2) and residues 89-97 (CDR-L3) according to the Kabat numbering system. In addition to the CDR loops, a fourth loop exists between CDR-2 and CDR-3 which is formed by framework 3 (Fwk3). FIG. 1A provides a representation of the framework 3 loop in the context of a Fab having a light and a heavy chain. FIGS. 1B and 1C also shows representative residues from these loops. The Kabat numbering system defines framework 3 as positions 66-94 in a heavy chain and positions 57-88 in a light chain.

Thus, in one embodiment, an antibody of the present invention comprises a variable (V) domain and an insert polypeptide, wherein the insert polypeptide is within positions 66-94 when the V domain is a VH domain, or positions 57-88 when the V domain is a VL domain, in accordance with the Kabat numbering system.

In some embodiments, the insert polypeptide is between amino acid residues 73 and 76 of the VH domain, in accordance with the Kabat numbering system. Preferably, the insert polypeptide may be between amino acid residues 73 and 74, 74 and 75, or 75 and 76 of the VH domain, in accordance with the Kabat numbering system. In some embodiments, the insert polypeptide is between amino acid residues 76 and 77, or 77 and 78 of the VH domain, in accordance with the Kabat numbering system. In some embodiments, one or more amino acid residues of the VH domain are replaced by the insert polypeptide.

In some embodiments, the insert polypeptide is between amino acid residues 67 and 70 of the VL domain, in accordance with the Kabat numbering system. Preferably, the insert polypeptide may be between amino acid residues 67 and 68, 68 and 69, or 69 and 70 of the VL domain, in accordance with the Kabat numbering system. In some embodiments, one or more amino acid residues of the VL domain are replaced by the insert polypeptide.

The antibody of the present invention may further comprise constant region domains. The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when antibody effector functions are required. Alternatively, IgG2 and IgG4 isotypes may be used when antibody effector functions are not required. It will be appreciated that sequence variants of these constant region domains may also be used. For example, IgG4 molecules in which the serine at position 241 has been changed to proline as described in Angal et al. Molecular Immunology, 1993, 30 (1), 105-108 may be used. It will also be understood by one skilled in the art that antibodies may undergo a variety of post-translational modifications. The type and extent of these modifications often depends on the host cell line used to express the antibody as well as the culture conditions.

Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995).

Antibodies for use in the present invention may be fully human antibodies, humanised antibodies, or chimeric antibodies.

In one embodiment, the antibody of the present invention is not a murine antibody. The term “murine antibody” refers to an antibody having heavy and light chains derived exclusively from murine B cells. This antibody is thus constituted of murine amino acid sequences, whatever the origin of the cell which enables the production thereof.

In one embodiment, the antibody of the present invention is a chimeric antibody. The term “chimeric antibody” refers to an antibody or antigen-binding fragment thereof wherein each light chain and/or each heavy chain sequence of which it is constituted comprises or consists of a hybrid sequence derived from at least two different animals, notably mammals, in particular, an antibody that combines the rabbit, murine, rat, or cynomolgus variable region with the human constant region.

In one embodiment, the antibody of the present invention is a humanised antibody. Humanised antibodies are antibody molecules from non-human species having one or more complementarity determining regions (CDRs) from the non-human species and a framework region from a human immunoglobulin molecule (see, for example, U.S. Pat. No. 5,585,089). In particular, the term “humanised” refers to an antibody or antigen-binding fragment thereof wherein the heavy and/or light chain contains one or more CDRs from a non-human antibody (such as a rabbit, murine, rat, cynomolgus or llama monoclonal antibody) grafted into a heavy and/or light chain variable region framework of an acceptor antibody such as a human antibody. Rather than the entire CDR being transferred, only one or more of the specificity determining residues from any one of the CDRs described herein above can be transferred to the human antibody framework (see for example, Kashmiri et al., 2005, Methods, 36, 25-34). Selected framework residues of the humanised antibody can be substituted with corresponding residues (so-called donor residues) from a non-human antibody (e.g. the antibody from which the CDRs or the specificity determining residues are derived) e.g. to restore or improve the antibody specificity or affinity.

Fully human antibodies are those antibodies in which the variable region(s) and the constant region(s) (where present) of both the heavy and/or the light chains are all of human origin, or substantially identical to sequences of human origin, not necessarily from the same antibody. Examples of fully human antibodies may include antibodies produced for example by phage display methods and antibodies produced by mice in which the murine immunoglobulin variable and/or constant region genes have been replaced by their human counterparts, e.g. as described in general terms in EP0546073 B1, U.S. Pat. Nos. 5,545,806, 5,569,825, 5,625,126, 5,633,425, 5,661,016, 5,770,429, EP0438474 B1 and EP0463151 B1.

The antibodies comprising a V domain and an insert polypeptide as disclosed herein may be further modified, for example chemically conjugated to further compounds which may affect e.g. its pharmacokinetic properties or antigen binding.

In one example the antibodies of the present invention are attached to poly(ethyleneglycol) (PEG) moieties. Antibodies linked to PEG may display further increased serum half-life, for example. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example, the antibody of the present invention is a modified Fab fragment wherein the modification is the addition to the C-terminal end of its heavy chain of one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules may be covalently linked through a thiol group of at least one cysteine residue located in the antibody fragment. Each polymer molecule attached to the modified antibody fragment may be covalently linked to the sulphur atom of a cysteine residue located in the antibody fragment. The covalent linkage will generally be a disulphide bond or, in particular, a sulphur-carbon bond. Where a thiol group is used as the point of attachment, appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, AL, USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

Preferably, the antibody of the present invention has a binding affinity (as measured by its dissociation constant K_{D}) for its cognate antigen of 10⁻⁵ M or less, 10⁻⁶ M or less, 10⁻⁷ M or less, 10⁻⁸ M or less, 10⁻⁹ M or less, 10⁻¹⁰M or less, or 10⁻¹¹ M or less. Affinity can be measured by known techniques such as surface plasmon resonance techniques including Biacore™ Affinity may be measured at room temperature, 25° C. or 37° C. Affinity may be measured at physiological pH, i.e. at about pH 7.4.

It will be appreciated that the affinity of antibodies provided by the present invention may be altered using any suitable method known in the art. The present invention therefore also relates to variants of the antibody molecules of the present invention, which have an improved affinity for albumin. Such variants can be obtained by a number of affinity maturation protocols including mutating the CDRs (Yang et al., J. Mol. Biol, 254. 392-403, 1995), chain shuffling (Marks et al., Bio/Technology, 10, 779-783, 1992), use of mutator strains of E. coli (Low et al., J. Mol. Biol., 260. 359-368, 1996), DNA shuffling (Patten et al., Curr. Opin. BiotechnoL, 8, 724-733, 1997), phage display (Thompson et al., J. Mol. Biol, 256. 77-88, 1996) and sexual PCR (Crameri et al. Nature, 391. 288-291, 1998).

### Human Serum Albumin-Binding Antibody

In one embodiment, the antibody of the invention comprises a V domain which binds to human serum albumin (HSA). In such an embodiment, the antibody may be useful in extending the serum half-life of the insert polypeptide. It will be appreciated that the V domain which binds albumin may do so alone, or co-operatively with another V domain, for example as a complementary VH/VL pair. Accordingly, in some embodiments, the antibody comprising a V domain binds albumin co-operatively with a complementary VH or VL domain. In one embodiment, the V domain is a VH domain, and the antibody of the invention further comprises a VL domain, wherein the VH binds albumin co-operatively with the VL domain as a VH/VL pair, i-e the antibody of the invention comprises a VH domain and binds to albumin co-operatively with a complementary VL. In one embodiment, the V domain is a VL domain, and the antibody of the invention further comprises a VH domain, wherein the VL binds albumin co-operatively with the VH domain as a VH/VL pair, i-e the antibody of the invention comprises a VL domain and binds to albumin co-operatively with a complementary VH. Where the antibody of the invention binds albumin, the VH/VL is typically termed “albumin binding domain”. As such, the albumin binding domain of the antibody of the invention refers to the VH, VL, or pair of VH/VL which binds to albumin.

In one embodiment, the antibody of the present invention binds cynomolgus serum albumin, murine serum albumin and/or rat serum albumin.

In one embodiment, the antibody of the present invention comprising a V domain which binds to human serum albumin is humanised. In one embodiment, the antibody of the present invention comprising an albumin binding domain is a humanised antibody wherein the heavy and/or light chain contains one or more CDRs from a rabbit, murine, or rat monoclonal antibody grafted into a heavy and/or light chain variable region framework of a human antibody. In one embodiment, the antibody of the present invention comprising an albumin binding domain is a humanised antibody wherein the heavy and/or light chain contains one or more CDRs from a rabbit antibody grafted into a heavy and/or light chain variable region framework of a human antibody, and wherein the heavy and/or light chain variable region framework comprises at least one amino acid substitution, such as an amino acid which is a donor residue.

In one embodiment, the antibody of the present invention, comprising a V domain which binds to human serum albumin, comprises a light and/or heavy chain sequence as shown below. (Variable domain underlined; CDRs in bold and italics.) In one embodiment, the antibody of the present invention, comprising a V domain which binds to human serum albumin, comprises a light and/or heavy chain sequence selected from below. In one embodiment, the antibody of the present invention, comprising a V domain which binds to human serum albumin, comprises a light and/or heavy chain variable domain sequence selected from below. In one embodiment, the antibody of the present invention, comprising a V domain which binds to human serum albumin, comprises at least one of CDR-L1, CDR-L2, and CDR-L3 sequence and/or at least one of CDR-H1, CDR-H2, and CDR-H3 sequence selected from below. In one embodiment, the antibody of the present invention, comprising a V domain which binds to human serum albumin, comprises a light and/or heavy chain sequence; and/or a light and/or heavy chain variable domain sequence; and/or at least one of CDR-L1, CDR-L2, and CDR-L3 sequence; and/or at least one of CDR-H1, CDR-H2, and CDR-H3 sequence selected from below.

CA645 Light Chain (gL5)
(SEQ ID NO: 1)
DIQMTQSPSSVSASVGDRVTITCQSSPSVWSNFLSWYQQKPGKAPKLLI
YEASKLTSGVPSRFSGSGSGTDFTLTISSLQPEDFATYYCGGGYSSISD
TTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYA
CEVTHQGLSSPVTKSFNRGEC
VL domain (gL5):
(SEQ ID NO: 2)
DIQMTQSPSSVSASVGDRVTITCQSSPSVWSNFLSWYQQKPGKAPKLLI
YEASKLTSGVPSRFSGSGSGTDFTLTISSLQPEDFATYYCGGGYSSISD
TTFGGGTKVEIK
CDR-L1:
(SEQ ID NO: 3)
QSSPSVWSNFLS
CDR-L2:
(SEQ ID NO: 4)
EASKLTS
CDR-L3:
(SEQ ID NO: 5)
GGGYSSISDTT
CA645 Heavy Chain (gH5)
(SEQ ID NO: 6)
EVQLLESGGGLVQPGGSLRLSCAVSGIDLSNYAINWVRQAPGKGLEWIG
IIWASGTTFYATWAKGRFTISRDNSKNTVYLQMNSLRAEDTAVYYCART
VPGYSTAPYFDLWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSC
VH domain (gH5):
(SEQ ID NO: 7)
EVQLLESGGGLVQPGGSLRLSCAVSGIDLSNYAINWVRQAPGKGLEWIG
IIWASGTTFYATWAKGRFTISRDNSKNTVYLQMNSLRAEDTAVYYCART
VPGYSTAPYFDLWGQGTLVTVSS
CDR-H1:
(SEQ ID NO: 8)
GIDLSNYAIN
CDR-H2:
(SEQ ID NO: 9)
IIWASGTTFYATWAKG
CDR-H3:
(SEQ ID NO: 10)
TVPGYSTAPYFDL

In some embodiments, the antibody of the present invention comprises variants of the VL and VH domains which bind human serum albumin as described above (SEQ ID NO: 2 and 7 respectively) that comprise an additional cysteine residue such that a disulphide bond may be formed between the VL and VH domains. The additional cysteine-containing variants may have the following sequences (wherein the additional cysteine residues are underlined):

CA645-Cys VL domain (gL5):
(SEQ ID NO: 102)
DIQMTQSPSSVSASVGDRVTITCQSSPSVWSNFLSWYQQKPGKAPKLLI
YEASKLTSGVPSRFSGSGSGTDFTLTISSLQPEDFATYYCGGGYSSISD
TTFGCGTKVEIK
CA645-Cys VH domain (gH5):
(SEQ ID NO: 103)
EVQLLESGGGLVQPGGSLRLSCAVSGIDLSNYAINWVRQAPGKCLEWIG
IIWASGTTFYATWAKGRFTISRDNSKNTVYLQMNSLRAEDTAVYYCART
VPGYSTAPYFDLWGQGTLVTVSS

Additional examples of VL and VH domains which bind human serum albumin, and which may be used in the antibody of the invention comprise SEQ ID NO: 105, 106, 107, 108 and 109 as described below (CDRs in bold and italics).

CA645 VH domain (gH1):
(SEQ ID NO: 105)
EVQLLESGGGLVQPGGSLRLSCAVSGIDLSNYAINWVRQAPGKGLEWIG
IIWASGTTFYATWAKGRFTISRDSTTVYLQMNSLRAEDTAVYYCARTVP
GYSTAPYFDLWGQGTLVTVSS
CA645 VH domain (gH37):
(SEQ ID NO: 106)
EVQLLESGGGLVQPGGSLRLSCAVSGIDLSNYAINWVRQAPGKGLEWIG
IIWASGTTAYATWAKGRFTISRDNSKNTVYLQMNSLRAEDTAVYYCART
VPGYSTAPYFDLWGQGTLVTVSS
CA645 VH domain (gH47):
(SEQ ID NO: 107)
EVQLLESGGGLVQPGGSLRLSCAVSGIDLSNYAINWVRQAPGKGLEWIG
IIWASGTTFYATWAKGRFTISRDNSKNTVYLQMNSLRAEDAVYYCARTV
PGYSAAPYFDLWGQGTLVTVSS
CA645 VL domain (gL1):
(SEQ ID NO: 108)
DIVMTQSPSSVSASVGDRVTITCQSSPSVWSNFLSWYQQKPGKAPKLLI
YEASKLTSGVPSRFKGSGSGTDFTLTISSLQPEDFATYYCGGGYSSISD
TTFGGGTKVEIK
CA645 VL domain (gL4):
(SEQ ID NO: 109)
DIQMTQSPSSVSASVGDRVTITCQSSPSVWSNFLSWYQQKPGKAPKLLI
YEASKLTSGVPSRFSGSGSGTDFTLTISSLQPEDFATYYC GGGYSSIS
D TTFGGGTKVEIK

In some embodiments, the antibody of the present invention comprises variants of the VL and VH domains which bind human serum albumin as described above (SEQ ID NO: 105 to SEQ ID NO: 109) that comprise an additional cysteine residue such that a disulphide bond may be formed between the VL and VH domains. The additional cysteine-containing variants may have the following sequence (wherein the additional cysteine residue is underlined):

CA645-Cys VL (gL4):
(SEQ ID NO: 110)
DIQMTQSPSSVSASVGDRVTITCQSSPSVWSNFLSWYQQKPGKAPKLLI
YEASKLTSGVPSRFSGSGSGTDFTLTISSLQPEDFATYYCGGGYSSISD
TTFGCGTKVEIKRT

In some embodiments, the VH framework of the albumin binding domain is human (for example VH3, such as VH3 1-3 3-23), and comprises for example 1, 2, 3, 4, 5 or 6 amino acid substitutions, such as amino acids which are donor residues. In such embodiments, the VH may have a sequence shown in SEQ ID NO: 7, SEQ ID NO: 103, SEQ ID NO: 105, SEQ ID NO: 106, SEQ ID NO: 107 or a variant of any one of the same with at least 95, 96, 97, 98 or 99% similarity of identity.

In some embodiments, the VL framework of the albumin binding domain is human (for example VK1, such as 2-1- (1) L5), and comprises for example 1, 2, 3, 4, 5 or 6 amino acid substitutions, such as amino acids which are donor residues. In such embodiments, the VL may have a sequence shown in SEQ ID NO: 2, SEQ ID NO: 102, SEQ ID NO: 108, SEQ ID NO: 109, SEQ ID NO: 110, or a variant of any one of the same with at least 95, 96, 97, 98 or 99% similarity of identity.

In some embodiments, the albumin binding domain comprises VH and VL sequences selected from the combinations SEQ ID NO: 7&2, 7&102, 7&108, 7&109, 7&110, 103&2, 103&102, 103&108, 103&109, 103&110, 105&2, 105&102, 105&108, 105&109, 105&110, 106&2, 106&102, 106&108, 106&109, 106&110, 107&2, 107&102, 107&108, 107&109, 107&110 or a variant or variants of any of the same with at least 95, 96, 97, 98 or 99% similarity or identity.

In some embodiments, the VL and VH sequences of the albumin binding domain are SEQ ID NO:102 and SEQ ID NO: 103, respectively. In some embodiments, the VL and VH sequences of the albumin binding domain are SEQ ID NO:2 and SEQ ID NO: 7, respectively.

In one embodiment, the antibody comprising a V domain which binds human serum albumin is a Fab or scFv. The VL and VH domains of SEQ ID NOs: 2 and 7 are preferred for the Fab format. In one embodiment, the antibody comprising a V domain which binds human serum albumin is a scFv which binds albumin cooperatively with a complementary V (VL or VH) domain. In such embodiment, the scFv may comprise any one of the VH domains and any one of the VL domains as described above, linked in the VH-VL or VL-VH orientation by a (G4S)₄ linker (SEQ ID NO:16). The VL and VH domains of SEQ ID NOs: 2 and 7 are preferred for the scFv format. The VL domain of SEQ ID NO: 2 may comprise the additional two amino acids RT at the C-terminus. In one embodiment, the antibody comprising a V domain which binds human serum albumin is a scFv which comprises the VL and VH domains of SEQ ID NOs: 2 and 7 separated by a (G4S)₄ linker. In one example, the scFv is VH-VL oriented. In another example, the scFv is VL-VH oriented. In one embodiment, the antibody comprising a V domain which binds human serum albumin is a scFv which comprises or has the SEQ ID NO: 143 as shown below. In one particular embodiment, the antibody comprising a V domain which binds human serum albumin is a dsscFv. In one embodiment, the antibody comprising a V domain which binds human serum albumin is a dsscFv which binds albumin cooperatively with a complementary V (VL or VH) domain. In such embodiment, the dsscFv may comprise any one of the VH domains and any one of the VL domains as described above, comprising, if not already present, an additional cysteine residue such that a disulphide bond may be formed between the VL and VH domains, wherein the VL and VH domains are linked in the VH-VL or VL-VH orientation by a (G4S)₄ linker. The VL and VH domains of SEQ ID NOs: 102 and 103 are preferred for the dsscFv format. The VL domain of SEQ ID NO: 102 may comprise the additional two amino acids RT at the C-terminus. In one embodiment, the antibody comprising a V domain which binds human serum albumin is a dsscFv which comprises the VL and VH domains of SEQ ID NOs: 102 and 103 separated by a (G4S)₄ linker (SEQ ID NO:16). In one example, the dsscFv is VH-VL oriented. In another example, the dsscFv is VL-VH oriented. In one embodiment, the antibody comprising a V domain which binds human serum albumin is a dsscFv which comprises or has the SEQ ID NO: 144 as shown below.

CA645 scFv (VH-VL):
(SEQ ID NO: 143)
EVQLLESGGGLVQPGGSLRLSCAVSGIDLSNYAINWVRQAPGKGLEWIG
IIWASGTTFYATWAKGRFTISRDNSKNTVYLQMNSLRAEDTAVYYCART
VPGYSTAPYFDLWGQGTLVTVSSGGGGSGGGGSGGGGSGGGGSDIQMTQ
SPSSVSASVGDRVTITCQSSPSVWSNFLSWYQQKPGKAPKLLIYEASKL
TSGVPSRFSGSGSGTDFTLTISSLQPEDFATYYCGGGYSSISDTTFGQG
TKVEIK
CA645 dsscFv (VH-VL) wherein the additional
cysteine residues are underlined:
(SEQ ID NO: 144)
EVQLLESGGGLVQPGGSLRLSCAVSGIDLSNYAINWVRQAPGKCLEWIG
IIWASGTTFYATWAKGRFTISRDNSKNTVYLQMNSLRAEDTAVYYCART
VPGYSTAPYFDLWGQGTLVTVSSGGGGSGGGGSGGGGSGGGGSDIQMTQ
SPSSVSASVGDRVTITCQSSPSVWSNFLSWYQQKPGKAPKLLIYEASKL
TSGVPSRFSGSGSGTDFTLTISSLQPEDFATYYCGGGYSSISDTTFGCG
TKVEIK

In another embodiment, the antibody comprising a V domain which binds human serum albumin is a full-length IgG. The VL and VH domains of SEQ ID NOs: 2 and 7 are preferred for the IgG format. In one embodiment, the antibody comprising a V domain which binds human serum albumin is an IgG1. In one embodiment, the antibody comprising a V domain which binds human serum albumin is an IgG4. In one embodiment, the antibody comprising a V domain which binds human serum albumin is an IgG4P.

It will be appreciated that one or more (for example 1, 2, 3 or 4) amino acid substitutions, additions and/or deletions may be made to the CDRs or other sequences (e.g. variable domains) provided by the present invention without significantly altering the ability of the antibody to bind to its cognate antigen (e.g. HSA). The effect of any amino acid substitutions, additions and/or deletions can be readily tested by one skilled in the art. For example, one or more (for example 1, 2, 3 or 4) amino acid substitutions, additions and/or deletions may be made to the framework region employed in the antibody or fragment provided by the present invention, wherein binding affinity to the cognate antigen is retained or increased. In some embodiments, the amino acid at position 72 of the framework 3 of the VH domain is wild type amino acid, naturally present at position 72 of said VH domain.

As such, the present invention also includes antibodies which comprise sequences which are at least 90%, 91%, 92%, 93%, 94%, 95%, 96%, 97%, 98% or 99% similar or identical to a sequence given herein. “Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656).

The antibody of the present invention may bind to albumin with a binding affinity sufficient to extend the half-life of the insert polypeptide, such as a therapeutic protein or a second antibody, in vivo. In one embodiment, the insert polypeptide binds to HSA. In such embodiment, the insert polypeptide may bind to albumin with a binding affinity sufficient to extend the half-life of the scaffold antibody. It has been reported that a K_{D} for albumin of less than or equal to 2.5 μM will extend half-life in vivo (Nguyen, A. et al (2006) Protein Engineering, Design & Selection, 19(7), 291-297). In one example the antibody of the present invention may bind to albumin with a high binding affinity which is nanomolar or micromolar, for example a K_{D} equal to or less than 3 nM. Affinity may be measured using any suitable method known in the art, including surface plasmon resonance using natural or recombinant serum albumin.

In some embodiments, the insert polypeptide is an albumin binding peptide. Examples of albumin binding peptides are provided in WO2007/106120 and include:

| SEQ ID NO: | SEQUENCE |
|---|---|
| 111 | DLCLRDWGCLW |
| 112 | DICLPRWGCLW |
| 113 | MEDICLPRWGCLWGD |
| 114 | QRLMEDICLPRWGCLWEDDE |
| 115 | QGLIGDICLPRWGCLWGRSV |
| 116 | QGLIGDICLPRWGCLWGRSVK |
| 117 | EDICLPRWGCLWEDD |
| 118 | RLMEDICLPRWGCLWEDD |
| 119 | MEDICLPRWGCLWEDD |
| 120 | MEDICLPRWGCLWED |
| 121 | RLMEDICLARWGCLWEDD |
| 122 | EVRSFCTRWPAEKSCKPLRG |
| 123 | RAPESFVCYWETICFERSEQ |
| 124 | EMCYFPGICWM |

In one embodiment, the insert polypeptide is a second antibody which binds to HSA. In such embodiment, the insert polypeptide may have the features described above for the antibody comprising a V domain, i-e the scaffold antibody. In some embodiments, the insert polypeptide may comprise at least one of the CDRs, at least one of the VL or VH domains, or at least one of the light or heavy chains which bind albumin, as described above. In one embodiment, the insert polypeptide comprises at least one of the CDR-L1, CDR-L2 and CDR-L3 of sequence SEQ ID NO: 3, SEQ ID NO: 4 and SEQ ID NO: 5 respectively and/or one of the CDR-H1, CDR-H2 and CDR-H3 of sequence SEQ ID NO: 8, SEQ ID NO: 9 and SEQ ID NO: 10 respectively.

### Insert Polypeptide

In accordance with the present invention, the present disclosure is directed to antibodies comprising an insert polypeptide within a framework 3 region in a V domain. The V domain may be a VL or a VH domain. In one example, the antibody of the invention may comprise an insert polypeptide within a framework 3 region of both VL and VH, i-e the antibody of the invention may comprise two insert polypeptides, wherein one insert polypeptide is inserted into the framework 3 region of a VL and one insert polypeptide is inserted into the framework 3 region of a VH, and wherein the insert polypeptides are identical, similar or different. In one embodiment, the insert polypeptide is heterologous to the antibody. As used herein, “heterologous” generally means derived from a genotypically distinct entity from the rest of the entity to which it is being compared. In this context, it means that the inserted polypeptide would not normally be present in the native antibody sequence. In one embodiment, the insert polypeptide is a full-length protein. In one embodiment, the insert polypeptide is a fragment of a full-length protein. In one embodiment, the insert polypeptide is a functional fragment of a full-length protein. In one embodiment, the insert polypeptide is functional when expressed on its own. In one embodiment, the insert polypeptide is functional and/or retains functionality when present in the framework 3 region in a V domain of the antibody of the invention. Thus, in one embodiment, the present invention provides an antibody comprising a variable (V) domain and an insert polypeptide, wherein the insert polypeptide is within the framework 3 (Fwk3) region of the V domain and wherein the insert polypeptide is functional and/or retains functionality when present in the framework 3 region in the V domain of the antibody of the invention.

In one embodiment, the insert polypeptide comprises at least 10, 15, 20, 25, 30, 35, 40, 45, 50, 55, 60, 65, 70, 75, 80, 85, 90, 95, 100, 150, or 200 amino acids. In one embodiment, the insert polypeptide comprises less than 250, 300 or 350 amino acids. In one embodiment, the insert polypeptide comprises less than 250 amino acids. In one embodiment, the insert polypeptide comprises at least 10, 15, 20, 25, 30, 35, 40, 45, 50, 55, 60, 65, 70, 75, 80, 85, 90, 95, 100, 150, or 200 amino acids and less than 250 amino acids. In one embodiment, the insert polypeptide comprises between 50 and 250, preferably 100 and 250, more preferably between 150 and 250 amino acids.

In one embodiment, the insert polypeptide may be a therapeutic or diagnostic polypeptide. Suitable therapeutic polypeptides may include, for example, receptor agonists or antagonists, enzyme inhibitors, metal chelators, anti-viral agents, anti-fungal agents, cardiovascular drugs and chemotherapeutic drugs. In some embodiments, the insert polypeptide is a cytokine. The cytokine may be selected from, for example, IL-10, IL-15, IL-2, G-CSF, GM-CSF, EPO. In other embodiments, the insert polypeptide is sclerostin. In some embodiments, the insert polypeptide comprises more than one polypeptide sequence such as two copies of the same polypeptide, optionally linked. Advantageously in such embodiment, the insert polypeptide may form a multimer, in particular a functional multimer. In some embodiments the insert polypeptide comprises two, three or four polypeptide sequences and is able to form a dimer, trimer or tetramer of the polypeptide. In some embodiments. the insert polypeptide forms a homodimer, a homotrimer or a homotetramer. In some embodiments, the insert polypeptide comprises at least two cytokines. In one embodiment, the insert polypeptide comprises two human IL-10 sequences, optionally separated by a peptide linker, e.g. (G4S)₃ (SEQ ID NO: 15), i-e two human IL-10 sequences are inserted into the Fwk3 region of a V domain of the antibody of the invention. In such embodiment, the two human IL-10 sequences form an homodimer of IL-10 incorporated into the antibody of the invention.

In some embodiments, the insert polypeptide is a chemokine. The chemokine may be selected from, for example, CCLs (C-C motif chemokine ligands), CXCLs (CXC motif chemokine ligands). In some embodiments, the insert polypeptide is a hormone. The hormone may be selected from, for example, insulin or leptin. In some embodiments, the insert polypeptide is a growth factor.

It has been surprisingly found by the present inventors that insertion of a polypeptide into an antibody in the framework 3 loop region of a V domain does not significantly hinder the functionality of either the antibody or the insert polypeptide. In particular, the examples herein show that specific and cognate binding to the respective binding partners of both the antibody and the insert polypeptide is seen for several tested fusion protein constructs.

In some embodiments, the antibody of the invention binds its cognate antigen by said V domain with a binding affinity which is at least 50%, 60%, 70%, 80% or 90% when compared to the antibody without the insert polypeptide present. It will be appreciated that the V domain which binds the antigen may do so alone, or co-operatively with another V domain depending on the antibody format, for example as a complementary VH/VL pair. Accordingly, the antibody comprising a V domain may bind its antigen co-operatively with a complementary V (VH or VL) domain. In some embodiments, the antibody of the invention binds its cognate antigen with a binding affinity which is at least 50%, 60%, 70%, 80% or 90% when compared to the antibody without the insert polypeptide present. In some embodiments, the antibody of the invention comprises a VH domain and binds its antigen co-operatively with a complementary VL with a binding affinity which is at least 50%, 60%, 70%, 80% or 90% when compared to the antibody without the insert polypeptide present. In some embodiments, the antibody of the invention comprises a VL domain and binds its antigen co-operatively with a complementary VH with a binding affinity which is at least 50%, 60%, 70%, 80% or 90% when compared to the antibody without the insert polypeptide present. In some embodiments, the insert polypeptide part of the antibody binds its cognate binding partner with a binding affinity which is at least 50%, 60%, 70%, 80% or 90% when compared to the free polypeptide when not linked to the antibody.

Therefore, in one embodiment, the antibody of the invention comprises a variable (V) domain and an insert polypeptide, wherein the insert polypeptide is within the framework 3 (Fwk3) region of the V domain and wherein both the antibody comprising a V domain and the insert polypeptide are functional and/or retain functionality when the insert polypeptide is present. In such embodiments, the antibody of the invention has functionality of both the scaffold antibody and the insert polypeptide. Advantageously, the antibody of the invention may exercise simultaneously the functionality of both the scaffold antibody and the insert polypeptide.

The antibody comprising a variable (V) domain is considered functional and/or retaining functionality when the insert polypeptide is present in the framework 3 region of the V domain, where the antibody of the invention exhibits at least 50%, 60%, 70%, 80% or 90% of at least one of the functions of the antibody comprising a variable (V) domain without the insert polypeptide present. The insert polypeptide is considered functional and/or retaining functionality when it is present in the framework 3 region of the V domain, where the insert polypeptide exhibits at least 50%, 60%, 70%, 80% or 90% of at least one of the functions of the insert polypeptide when expressed on its own, i-e when compared to the free polypeptide when not linked to the antibody.

Functional or functionality of the antibody and/or the insert polypeptide refers to at least one biological activity of said antibody and/or insert polypeptide including for example target binding (e.g. antigen binding), binding affinity, cross-reactivity, neutralisation, half-life in serum, activation of cell surface proteins, e.g. T cell surface receptors. Methods for measuring functionality, e.g. binding affinity, neutralisation and activation of target cells are well known in the art and include for example the methods described in the Examples provided herein. For example, standard assays such as Scatchard analysis, or surface plasmon resonance technique (e.g. using BIACORE®) may be used to determine target binding (e.g. amigen binding) and binding affinity.

Surprisingly, the V domain (VL or VH domain) of the antibody of the invention can still form a functional binding domain with its corresponding or complementary V domain (VH or VL), where present.

In some embodiments, the insert polypeptide may be a second antibody, or binding fragment thereof. In such embodiments, the insert polypeptide may have any of the features of antibodies as described above. In particular, in some embodiments, the insert polypeptide is a scFv, a dsscFv, a single domain antibody such as a VH or VL or VHH. In some embodiments, the insert polypeptide may be a fully human, humanised, or chimeric antibody. The insert polypeptide may be comprised of one or more CDRs or may consist of one or more CDRs. For example, the insert polypeptide may comprise or consist of at least one of CDR-H1, CDR-H2, CDR-H3 and/or CDR-L1, CDR-L2, CDR-L3, optionally linked.

It will be appreciated by those skilled in the art that this would function as a bivalent antibody, or bispecific antibody. Thus, in one aspect, the invention provides a novel bivalent or bispecific antibody format, capable of simultaneous binding two different epitopes. In some aspects, the epitopes are distinct epitopes present on the same antigen. In another aspect, the invention provides a novel bispecific antibody format capable of simultaneous binding two different antigens.

In one embodiment, an antigen of interest bound by the first or the second antibody or antibody fragment may be a cell-associated protein, for example a cell surface protein on cells such as bacterial cells, yeast cells, T-cells, endothelial cells or tumour cells, or it may be a soluble protein. Antigens of interest may also be any medically relevant protein such as those proteins upregulated during disease or infection, for example receptors and/or their corresponding ligands. Particular examples of cell surface proteins include adhesion molecules, for example integrins such as β1 integrins e.g. VLA-4, E-selectin, P selectin or L-selectin, CD2, CD3, CD4, CD5, CD7, CD8, CDlla, CDllb, GDIS, CD19, CD20, CD23, CD25, CD28, CD33, CD38, CD40, CD45, CDW52, CD69, CD134 (OX40), ICOS, BCMP7, CD137, CD27L, CDCP1, DPCR1, DPCR1, dudulin2, FLJ20584, FLJ40787, HEK2, KIAA0634, KIAA0659, KIAA1246, KIAA1455, FTBP2, FTK, MAF2, MRP2, nectin-like2, NKCC1, PTK7, RAIG1, TCAM1, SC6, BCMP101, BCMP84, BCMP11, DTD, carcinoembryonic antigen (CEA), human milk fat globulin (HMFG1 and 2), MHC Class I and MHC Class II antigens, and VEGF, and where appropriate, receptors thereof. Soluble antigens include interleukins such as IL-1, IL-2, IL-3, IL-4, IL-5, IL-6, IL-8, IL-12, IL-16 or IL-17, viral antigens for example respiratory syncytial virus or cytomegalovirus antigens, immunoglobulins, such as IgE, interferons such as interferon α, interferon β or interferon γ, tumour necrosis factor-α, tumour necrosis factor-β, colony stimulating factors such as G-CSF or GM-CSF, and platelet derived growth factors such as PDGF-α, and PDGF-β and where appropriate receptors thereof. Other antigens include bacterial cell surface antigens, bacterial toxins, viruses such as influenza, EBV, Hep A, B and C, bioterrorism agents, radionuclides and heavy metals, and snake and spider venoms and toxins.

In some embodiments, neither the scaffold antibody nor the insert polypeptide binds the hapten (4-hydroxy-3-nitrophenyl) acetyl (NP) or hapten (4-hydroxy-5-iodo-3-nitrophenyl) acetyl (NIP). In some embodiments, the antibody is different from BI-8 murine antibody.

In one embodiment, the second antibody binds to a different antigen to the V domain of the antibody.

In other embodiments, the second antibody binds to the same antigen as the V domain of the antibody. Preferably, the second antibody binds to a different epitope compared to the V domain of the antibody. In such embodiments, the antibody may bind its cognate antigen with a binding affinity which is greater than when compared to the antibody without the second antibody present.

In one embodiment, the insert polypeptide is a second antibody which binds IL-17, in particular IL-17A. In one embodiment, the insert polypeptide is a second antibody which binds IL-17AA, IL-17AF, and IL-17FF. In one embodiment, the second antibody which binds IL-17 is a scFv. In one embodiment, the second antibody which binds IL-17 is a dsscFv. In one embodiment, the antibody of the present invention is a Fab fragment that binds to both HSA and IL-17, in particular the antibody comprising a V domain binds to HSA and the insert polypeptide is a dsscFv which binds IL-17 i.e. the scaffold antibody is a Fab fragment which binds HSA and the second antibody inserted into the Fwk3 region of the V domain of the scaffold antibody is a dsscFv which binds IL-17. In another embodiment, the antibody of the invention is a whole IgG that binds to both HSA and IL-17, in particular the antibody comprising a V domain binds to HSA and the insert polypeptide is a dsscFv which binds IL-17 i.e. the scaffold antibody is a full IgG which binds HSA and the second antibody inserted into the Fwk3 region of the V domain of the scaffold antibody is a dsscFv which binds IL-17. In one embodiment, the antibody of the invention is a Fab fragment which comprises or has the heavy and light chains of SEQ ID NO: 129 (CA645 Fab heavy chain with CA497 dsscFv (vHvL) inserted into framework 3) and SEQ ID NO: 1 respectively, or a variant or variants of any of the same with at least 90, 91, 92, 93, 94, 95, 96, 97, 98 or 99% similarity or identity provided that it retains its functionality, e.g. binding to HSA and/or IL-17. In one embodiment, the antibody of the invention is a Fab fragment which comprises or has the heavy and light chains of SEQ ID NO: 130 (CA645 Fab heavy chain with CA497 dsscFv (vLvH) inserted into framework 3) and SEQ ID NO: 1 respectively or a variant or variants of any of the same with at least 90, 91, 92, 93, 94, 95, 96, 97, 98 or 99% similarity or identity provided that it retains its functionality e.g. binding to HSA and/or IL-17. In one embodiment, the antibody of the invention is a whole IgG which comprises or has the heavy and light chains of SEQ ID NO: 131 (CA645 IgG4P heavy chain with CA497 dsscFv (vHvL) inserted into framework 3) and SEQ ID NO: 1 respectively, or a variant or variants of any of the same with at least 90, 91, 92, 93, 94, 95, 96, 97, 98 or 99% similarity or identity provided that it retains its functionality, e.g. binding to HSA and/or IL-17. In one embodiment, the antibody of the invention is a whole IgG which comprises or has the heavy and light chains of SEQ ID NO: 132 (CA645 IgG4P heavy chain with CA497 dsscFv (vLvH) inserted into framework 3) and SEQ ID NO: 1 respectively, or a variant or variants of any of the same with at least 90, 91, 92, 93, 94, 95, 96, 97, 98 or 99% similarity or identity provided that it retains its functionality, e.g. binding to HSA and/or IL-17.

In one embodiment, the antibody of the invention binds to both CD3 and CD28. In one embodiment, the antibody of the invention is a Fab fragment that binds to both CD3 and CD28, in particular the antibody comprising a V domain binds to CD3 and the insert polypeptide is a dsscFv which binds CD28 i.e. the scaffold antibody is a Fab fragment which binds CD3 and the second antibody inserted into the Fwk3 region of the V domain of the scaffold antibody is a dsscFv which binds CD28. In one embodiment, the antibody of the invention is a Fab fragment that binds to both CD3 and CD28, in particular the antibody comprising a V domain binds to CD28 and the insert polypeptide is a dsscFv which binds CD3 i.e. the scaffold antibody is a Fab fragment which binds CD28 and the second antibody inserted into the Fwk3 region of the V domain of the scaffold antibody is a dsscFv which binds CD3. In another embodiment, the antibody of the invention is a whole IgG that binds to both CD3 and CD28, in particular the antibody comprising a V domain binds to CD3 and the insert polypeptide is a dsscFv which binds CD28 i.e. the scaffold antibody is a full IgG which binds CD3 and the second antibody inserted into the Fwk3 region of the V domain of the scaffold antibody is a dsscFv which binds CD28. In another embodiment, the antibody of the invention is a whole IgG that binds to both CD3 and CD28, in particular the antibody comprising a V domain binds to CD28 and the insert polypeptide is a dsscFv which binds CD3 i.e. the scaffold antibody is a full IgG which binds CD28 and the second antibody inserted into the Fwk3 region of the V domain of the scaffold antibody is a dsscFv which binds CD3. In one embodiment, the antibody of the invention is a Fab fragment which comprises or has the heavy and light chains of SEQ ID NO: 139 (anti-CD28 Fab heavy chain with anti-CD3 dsscFv inserted into framework 3) and SEQ ID NO: 136 respectively, or a variant or variants of any of the same with at least 90, 91, 92, 93, 94, 95, 96, 97, 98 or 99% similarity or identity provided that it retains its functionality, e.g. binding to CD3 and/or CD28. In another embodiment, the antibody of the invention is a whole IgG which comprises or has the heavy and light chains of SEQ ID NO: 141 (anti-CD28 IgG4P heavy chain with anti-CD3 dsscFv inserted into framework 3) and SEQ ID NO: 136 respectively or a variant or variants of any of the same with at least 90, 91, 92, 93, 94, 95, 96, 97, 98 or 99% similarity or identity provided that it retains its functionality e.g. binding to CD3 and/or CD28.

Example sequences for use in the present invention include SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 105, SEQ ID NO: 106, SEQ ID NO: 107, SEQ ID NO: 108, SEQ ID NO: 109, SEQ ID NO: 110, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143 and SEQ ID NO: 144. Example combinations of heavy and light chains for use in the Fab fragments of the present invention include SEQ ID NO: 54 and SEQ ID NO: 1, SEQ ID NO: 56 and SEQ ID NO: 1, SEQ ID NO: 58 and SEQ ID NO: 1, SEQ ID NO: 60 and SEQ ID NO: 1, SEQ ID NO: 61 and SEQ ID NO: 1, SEQ ID NO: 63 and SEQ ID NO: 1, SEQ ID NO: 64 and SEQ ID NO: 1, SEQ ID NO: 66 and SEQ ID NO: 1, SEQ ID NO: 68 and SEQ ID NO: 1, SEQ ID NO: 70 and SEQ ID NO: 1, SEQ ID NO: 73 and SEQ ID NO: 1, SEQ ID NO: 74 and SEQ ID NO: 1, SEQ ID NO: 75 and SEQ ID NO: 1, SEQ ID NO: 76 and SEQ ID NO: 1, SEQ ID NO: 93 and SEQ ID NO: 1, SEQ ID NO: 94 and SEQ ID NO: 1, SEQ ID NO: 95 and SEQ ID NO: 1, SEQ ID NO: 101 and SEQ ID NO: 1, SEQ ID NO: 129 and SEQ ID NO: 1, SEQ ID NO: 130 and SEQ ID NO: 1, SEQ ID NO: 139 and SEQ ID NO: 136, SEQ ID NO: 140 and SEQ ID NO: 133. Example combinations of heavy and light chains for use in the whole IgG of the present invention include SEQ ID NO: 131 and SEQ ID NO: 1, SEQ ID NO: 132 and SEQ ID NO: 1, SEQ ID NO: 141 and SEQ ID NO: 136, SEQ ID NO: 142 and SEQ ID NO: 133.

### Linker Sequence

In some embodiments, the insert polypeptide is linked directly to the framework 3 region of the antibody. Linked directly as employed herein is intended to refer to the fact that the N- and C-terminal amino acids of the heterologous insert polypeptide would be linked by a peptide bond to the framework 3 region of the V domain of the antibody of the present invention.

In some embodiments, the antibody of the invention further comprises a linker sequence. In such embodiments, the linker sequence is preferably heterologous to both the antibody and insert polypeptide, that is the linker sequence would not normally be present in the native antibody sequence or insert polypeptide at that position.

In some embodiments, the linker sequence comprises at least one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty or thirty-one amino acids joining the N- and/or C-terminal end of the insert polypeptide to the framework 3 region. In some embodiments, the linker sequence comprises between one and thirty-one, or between five and thirty-one, or between six and twenty-eight, or between ten and twenty-six, or between fifteen and twenty-six, or between ten and twenty-one or between five and ten amino acids joining the N- and/or C-terminal end of the insert polypeptide to the framework 3 region.

In some embodiments, the linker sequence comprises at most thirty amino acids joining the N- and/or C-terminal end of the insert polypeptide to the framework 3 region.

In some embodiments, the linker at the N-terminus of the insert polypeptide is different in composition and/or length to the linker at the C-terminal end of the insert polypeptide. In some embodiments, the linker at the N-terminus of the insert polypeptide is identical or similar in composition and/or length to the linker at the C-terminal end of the insert polypeptide. In some embodiments, the linker sequence is derived from a human sequence. In some embodiments, the linker is a human linker. In some embodiments, the linker sequence is derived from a non-human sequence. In some embodiments, the linker is derived from a bovine sequence. In some embodiments, the linker is a bovine linker.

Examples of suitable linker regions for linking the insert polypeptide to the framework 3 region of a V domain include, but are not limited to, flexible linker sequences and rigid linker sequences. Flexible linker sequences include those disclosed in Huston et al., 1988, PNAS 85:5879-5883; Wright & Deonarain, Mol. Immunol., 2007, 44(11):2860-2869; Alfthan et al., Prot. Eng., 1995, 8(7):725-731; Luo et al., J. Biochem., 1995, 118(4):825-831; Tang et al., 1996, J. Biol. Chem. 271(26):15682-15686; and Turner et al., 1997, JIMM 205, 42-54 (see Table 1 below for representative examples).

TABLE 1
Examples of Flexible Linker Sequences

| SEQ ID NO: | SEQUENCE |
|---|---|
| 11 | SGGGGSE |
| 12 | DKTHTS |
| 13 | (S)GGGGS |
| 14 | (S)GGGGSGGGGS |
| 15 | (S)GGGGSGGGGSGGGGS |
| 16 | (S)GGGGSGGGGSGGGGSGGGGS |
| 17 | (S)GGGGSGGGGSGGGGSGGGGSGGGGS |
| 18 | AAAGSG-GASAS |
| 19 | AAAGSG-XGGGS-GASAS |
| 20 | AAAGSG-XGGGSXGGGS-GASAS |
| 21 | AAAGSG-XGGGSXGGGSXGGGS-GASAS |
| 22 | AAAGSG-XGGGSXGGGSXGGGSXGGGS-GASAS |
| 23 | AAAGSG-XS-GASAS |
| 24 | PGGNRGTTTTRRPATTTGSSPGPTQSHY |
| 25 | ATTTGSSPGPT |
| 26 | ATTTGS |
| — | GS |
| 27 | EPSGPISTINSPPSKESHKSP |
| 28 | GTVAAPSVFIFPPSD |
| 29 | GGGGIAPSMVGGGGS |
| 30 | GGGGKVEGAGGGGGS |
| 31 | GGGGSMKSHDGGGGS |
| 32 | GGGGNLITIVGGGGS |
| 33 | GGGGVVPSLPGGGGS |
| 34 | GGEKSIPGGGGS |
| 35 | RPLSYRPPFPFGFPSVRP |
| 36 | YPRSIYIRRRHPSPSLTT |
| 37 | TPSHLSHILPSFGLPTEN |
| 38 | RPVSPFTFPRLSNSWLPA |
| 39 | SPAAHFPRSIPRPGPIRT |
| 40 | APGPSAPSHRSLPSRAFG |
| 41 | PRNSIHFLHPLLVAPLGA |
| 42 | MPSLSGVLQVRYLSPPDL |
| 43 | SPQYPSPLTLTLPPHPSL |
| 44 | NPSLNPPSYLHRAPSRIS |
| 45 | LPWRTSLLPSLPLRRRP |
| 46 | PPLFAKGPVGLLSRSFPP |
| 47 | VPPAPVVSLRSAHARPPY |
| 48 | LRPTPPRVRSYTCCPTP- |
| 49 | PNVAHVLPLLTVPWDNLR |
| 50 | CNPLLPLCARSPAVRTFP |
| 77 | KYQSGGSGMG |
| 78 | SGGSYTY |
| 79 | GGGSGGGG |
| 80 | GSSSSGS |
| — | GGG |
| 81 | SGGGG |
| — | S |

(S) is optional in sequences 13 to 17.
X represents any residue.

In particular embodiments, the antibody comprises the linker sequences SEQ ID NOs: 106 and 107.

Examples of rigid linkers include the peptide sequences GAPAPAAPAPA (SEQ ID NO:51), PPPP (SEQ ID NO:52), PPP, IPFTV (SEQ ID NO: 82) and EYHGLQ (SEQ ID NO: 104). Further examples include sequences derived from the elongated stalk of CDRH3 of bovine antibodies (Wang et al., Cell, 2013, 153:1379-1393); preferable sequences include long linker sequences such as TSVHQETKKYQS (SEQ ID NO: 96) and SYTYNYEWHVDV (SEQ ID NO: 97) (comprising both solvent exposed and buried residues from a bovine CDH3 stalk); and short linker sequences such as ETKKYQS (SEQ ID NO: 98) and SYTYNYE (SEQ ID NO: 99) (comprising only the solvent exposed residues from a bovine CDH3 stalk).

In some embodiments, variants of any of the linker sequences disclosed herein may be used, for example, variants where in one, two, three, four or more amino acid residues have been substituted, inserted, or removed with respect to the original linker sequence. Variants of linker sequences may also include repeat variants, where the linker sequence is elongated by repeating the linker sequence. In one embodiment, a preferred linker sequence to be repeated is SEQ ID NO: 13, leading to the motif: (GₘS)ₙ, where m is 4 and n can be any integer (for example 1, 2 or 3). In other embodiments, m can be a different integer from 1 to 10.

Whether or not the insert polypeptide is linked directly or via a linker sequence, in some embodiments, one or more amino acids in the framework 3 region may be deleted as a result of linking the polypeptide insert. In some embodiments, the insert polypeptide is between amino acid residues 73 and 75 of the VH domain, in accordance with the Kabat numbering system and the amino acid in position 74 is deleted.

### Pharmaceutical Compositions and Methods of Use

Antibodies of the invention and as described herein are useful in the treatment of diseases or disorders including inflammatory diseases and disorders, immune diseases and disorders, fibrotic disorders and cancers.

The term “inflammatory disease” or “disorder” and “immune disease or disorder” includes rheumatoid arthritis, psoriatic arthritis, still's disease, Muckle Wells disease, psoriasis, Crohn's disease, ulcerative colitis, SLE (Systemic Lupus Erythematosus), asthma, allergic rhinitis, atopic dermatitis, multiple sclerosis, vasculitis, Type I diabetes mellitus, transplantation and graft-versus-host disease.

The term “fibrotic disorder” includes idiopathic pulmonary fibrosis (IPF), systemic sclerosis (or scleroderma), kidney fibrosis, diabetic nephropathy, IgA nephropathy, hypertension, end-stage renal disease, peritoneal fibrosis (continuous ambulatory peritoneal dialysis), liver cirrhosis, age-related macular degeneration (ARMD), retinopathy, cardiac reactive fibrosis, scarring, keloids, bums, skin ulcers, angioplasty, coronary bypass surgery, arthroplasty and cataract surgery.

The term “cancer” includes a malignant new growth that arises from epithelium, found in skin or, more commonly, the lining of body organs, for example: breast, ovary, prostate, lung, kidney, pancreas, stomach, bladder or bowel. Cancers tend to infiltrate into adjacent tissue and spread (metastasise) to distant organs, for example: to bone, liver, lung or the brain.

Thus, according to a further aspect of the invention, there is provided a pharmaceutical composition which comprises an antibody of the invention in association with one or more pharmaceutically acceptable carriers, excipients or diluents.

Also provided is the use of an antibody of the invention for the manufacture of a medicament for the treatment of a disease or disorder. Most preferably, the disease or disorder is an inflammatory disease or disorder.

Pharmaceutical compositions according to the invention may, for example, take a form suitable for parenteral, subcutaneous, or ophthalmic administration. Where appropriate, for example if the antibody binds to albumin via its V domain, it may be desirable to pre-formulate the antibody of the invention with human or recombinant serum albumin, using any suitable method known in the art.

Where the pharmaceutical formulation is a liquid, for example a solution or suspension, then the formulation may further comprise albumin, for example human serum albumin, in particular recombinant albumin such as recombinant human serum albumin. Suitable amounts may be in the range of less than 2% w/w of the total formulation, in particular less than 1, 0.5, or 0.1% w/w. This may assist in stabilizing the antibody component in the formulation. The pharmaceutical composition may be lyophilized for reconstitution later, with an aqueous solvent. In one embodiment there is provided a unit dose container, such as a vial, comprising a lyophilized antibody according to the invention.

The antibodies of the invention may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

The compositions may, if desired, be presented in a pack or dispenser device which may contain one or more unit dosage forms containing the active ingredient. The pack or dispensing device may be accompanied by instructions for administration.

The antibody formats of the invention can be delivered dispersed in a solvent, e.g., in the form of a solution or a suspension. It can be suspended in an appropriate physiological solution, e.g., saline or other pharmacologically acceptable solvent or a buffered solution. Buffered solutions known in the art may contain 0.05 mg to 0.15 mg disodium edetate, 8.0 mg to 9.0 mg NaCl, 0.15 mg to 0.25 mg polysorbate, 0.25 mg to 0.30 mg anhydrous citric acid, and 0.45 mg to 0.55 mg sodium citrate per 1 ml of water so as to achieve a pH of about 4.0 to 5.0. A suspension can employ, for example, lyophilised antibody.

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes.

This may include production and sterilization by filtration of the buffered solvent/solution used for the formulation, aseptic suspension of the antibody in the sterile buffered solvent solution, and dispensing of the formulation into sterile receptacles by methods familiar to those of ordinary skill in the art.

The quantity of an antibody of the invention required for the prophylaxis or treatment of a particular condition will vary depending on the antibody and the condition to be treated. In general, however, daily dosages may range from around 10 ng/kg to 1000 mg/kg, from 100 ng/kg to 100 mg/kg, or from 0.1 mg/kg to 10 mg/kg body weight.

The present invention also provides for methods and compositions for the delivery of the antibodies as described herein by gene therapy, particularly by adeno-associated virus (AAV) vector.

Hence, the present invention provides for a pharmaceutical composition comprising a viral vector having a viral capsid and an artificial genome comprising an expression cassette flanked by inverted terminal repeats (ITRs) wherein the expression cassette comprises a transgene comprising a polynucleotide sequence encoding the antibody as described herein. The ITRs sequences may be used for packaging the artificial genome comprising the polynucleotide sequences encoding the antibody as described herein into the virion of the viral vector.

The transgene in the expression cassette is operably linked to expression control elements such as promoters that will control expression of the transgene in human cells.

The viral vector is preferably AAV based viral vectors. A variety of AAV capsids have been described in the art. Methods of generating AAV vectors have also been described extensively in the literature (e.g., WO 2003/042397; WO 2005/033321, WO 2006/110689; U.S. Pat. No. 7,588,772 B2). The source of AAV capsids may be selected from an AAV which targets a desired tissue. For example, suitable AAV may include, e.g., AAV9 (U.S. Pat. No. 7,906,111; US2011-0236353-A1), rh10 (WO 2003/042397) and/or hu37 (U.S. Pat. No. 7,906,111B2; US20110236353). However, other AAV, including, e.g., AAV1, AAV2, AAV-TT, AAV3, AAV4, AAV5, AAV6, AAV7, AAV8 and AAV.PHP.B (or variants thereof) and others may also be selected.

Methods for generating and isolating AAV viral vectors suitable for delivery to a subject are known in the art (U.S. Pat. No. 7,790,449B2; U.S. Pat. No. 7,282,199B2; WO 2003/042397; WO 2005/033321, WO 2006/110689; and U.S. Pat. No. 7,588,772 B2).

The pharmaceutical composition comprising a viral vector having a viral capsid and an artificial genome according to the invention is designed to ensure that both the heavy and light chains are expressed. The polynucleotide sequences of the heavy and light chain of the antibodies according to the present invention may be engineered in a single construct and separated by, for example, a cleavable linker or internal ribosome entry site (IRES) element so that the heavy and light chains are separately expressed. In some embodiments, the cleavable linker is a self-cleaving furin/F2A linker (Fang et al., Nature Biotechnology 23: 584-590, 2005 and Fang et al. Mol Ther 15: 1153-9, 2007). For example, a furin/F2A linker may be incorporated into an expression cassette to separate the heavy and light chain polynucleotide coding sequences.

In some other embodiments, the polynucleotide sequence of the heavy and light chains of the antibodies according to the present invention are connected via a flexible, non-cleavable linker.

In some embodiments such linker may be a flexible peptide linker such as those encoding scFv (composed of Gly and Ser) so that the heavy and light chain domains are free to move relative to one another.

The invention will now be described with reference to the following examples, which are merely illustrative and should not in any way be construed as limiting the scope of the present invention.

### Examples

### General Methodology

### Framework 3 Insertion

In addition to three CDR loops, antibody light and heavy chains, both conventional and single-chain camelid VHH, have a fourth loop which is formed by framework 3 (FIG. 1A). The Kabat numbering system defines framework 3 as positions 66-94 in a heavy chain and positions 57-88 in a light chain.

Gene Design and Transient Cell Expression Genes encoding light and heavy chain V-regions, including variants with a framework 3 insertion, were designed and constructed by an automated synthesis approach (ATUM). The genes were cloned into expression vectors containing DNA encoding human Cx region (Km3 allotype) and heavy chain 71 CH1 region, respectively. Heavy and light chain expression vectors were then co-transfected into HEK293 cells and the recombinant antibody/Fab fusion protein molecules screened using an SPR binding assay.

The light V-region or light chain of the CA645 Fab fusion proteins described in the following Examples comprised or had SEQ ID NO: 2 (CA645 VL domain (gL5)) or SEQ ID NO: 1 (CA645 light chain gL5) respectively. Alternative light chains or light V-regions may be used, for example light V-regions comprising the VL domain of SEQ ID NO: 108 or SEQ ID NO: 109.

### Determination of Antibody/Fab Fusion Protein Binding Potency by Surface Plasmon Resonance

All surface plasmon resonance (SPR) experiments were carried out at 25° C. on a Biacore 3000 system using a pH 7.4 running buffer containing 10 mM HEPES, 150 mM NaCl, EDTA 2 mM and 0.005% (v/v) P20 (HBS-EP buffer).

Polyclonal goat F(ab)₂ fragment anti-human F(ab)₂, (Jackson Labs product code #109-006-097) was immobilized to the surface of CM5 sensor chips (GE Healthcare) by amine coupling. Briefly, the carboxymethyl dextran surface was activated with a fresh 1:1 (v/v) mixture of 50 mM N-hydroxysuccimide and 200 mM 1-ethyl-3-(3-dimethylaminopropyl)-carbodiimide for 5 minutes at a flow rate of 10 μL/min. The anti-human F(ab)₂ in 10 mM acetate pH 5.0 buffer was covalently coupled to chips at 50 μg/mL in a 10 minute pulse at the same flow rate. Finally the surface was deactivated with a 10 minute pulse of 1 M ethanolamine.HCl pH 8.5. A reference flow cell was prepared on the chip by omitting the protein from the above procedure.

Supernatants containing Fab-loop insert constructs, wild type Fab or control supernatants were diluted 1 in 5 in HPS-EP buffer prior to testing for binding of their respective ligand or receptor.

Human serum albumin (HSA) was supplied by Jackson ImmunoResearch (catalog #009-000-051) and was diluted in HBS-EP buffer to 1000 nM. The following cytokines and receptors were obtained from R&D Systems Ltd.: IL-15, IL-15-Rα-hFc, IL-2, IL-2-sRβ, G-CSF, G-CSF-Rα, GM-CSF, GM-CSF-Rα, EPO, EPO-R, IL-6, LRP4(ecd)-hFc, LRP6(ecd)-hFc, Lysozyme, KLK5. These proteins were reconstituted at typically 100 μg/mL and stored at −20° C. For experimental purposes they were freshly diluted in HBS-EP buffer to typically 50, 100 or 200 nM.

A typical SPR cycle comprised a flow rate of 10 μL/min with HBS-EP as running buffer and diluted supernatant was injected for 1 min to capture the antibody/Fab fusion protein. This was followed by a 30 μl injection of the diluted cognate ligand or receptor or buffer control. The subsequent dissociation phase was monitored for at least 3 min. In some experiments an additional 30 μL injection of the relevant cognate antigen of the antibody portion was included, HSA in the case of fusion protein constructs using CA645 Fab as the antibody polypeptide portion. In some experiments a specificity control cycle was included where the cognate receptor/ligand (the insert portion of the antibody/Fab fusion protein) was pre-mixed with a 2-fold molar excess of its binding partner. Each cycle terminated with a regeneration step comprising two 10 μL injections of 40 mM HCl interspersed with a 5 μL injection of 5 mM NaOH.

Report points were collected 10 s before supernatant and ligand/receptor injections for measuring fusion protein construct capture and at 5 s before and 15 s after the end of the ligand/receptor injection for assessing binding of cognate ligand/receptor. Report point and sensorgram data collected represented the difference in resonance units (RU) between the polyclonal capture flow cell and the reference flow cell.

Report point and sensorgram data were processed by subtracting the respective control cycle from the ligand/receptor cycle to account for any slow dissociation of the fusion protein construct from the capture surface. In every experiment the respective wild type Fab supernatant was included to check for any non-specific binding of ligand/receptor. If significant this was subtracted from the above.

In examples where dissociation constants (K_{D}) were estimated, the above method was modified by increasing the flow rate to 30 μl/min. Following capture of the Fab construct, 90 μl of the cognate ligand or buffer control was injected followed by a dissociation phase of 180 sec. A ligand binding over a range of concentrations (0.1, 0.2, 0.5, 1, 2, 5, & 10 nM) was tested. Sensorgrams were corrected for drift by subtraction of a zero ligand sensorgram and for non-specific interaction by subtraction of the wild type Fab corrected sensorgram. Affinity constants were calculated from separate dissociation and association rate constants that were fitted to the corrected sensorgrams using the Langmuir model of the BIAevaluation software (version 4.1.1).

### Example 1—CA645 Fab with IL-15 Insert Polypeptide

Interleukin 15 (IL-15), is a cytokine which plays a role in the immune response, and functions to promote the proliferation of T cells and natural killer (NK) cells. It is structurally similar to IL-2, and its receptor shares common chains with the IL-2 receptor.

A fusion protein was generated and expressed in accordance with the above methods to insert the human IL-15 sequence into the framework 3 region of the CA645 Fab construct, as shown below.

Human IL-15 sequence:
(SEQ ID NO: 53)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFIN
CA645 Fab heavy chain with human IL-15 (bold and
italics) inserted into framework 3, linkers
underlined:
(SEQ ID NO: 54)
EVQLLESGGGLVQPGGSLRLSCAVSGIDLSNYAINWVRQAPGKGLEWIGI
IWASGTTFYATWAKGRFTISRDNKYQSGGSGMGNWVNVISDLKKIEDLIQ
SMHIDATLYTESDVHPSCKVTAMKCFLLELQVISLESGDASIHDTVENLI
ILANNSLSSNGNVTESGCKECEELEEKNIKEFLQSFVHIVQMFINSGGSY
TYKNTVYLQMNSLRAEDTAVYYCARTVPGYSTAPYFDLWGQGTLVTVSSA
STKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPK
SC

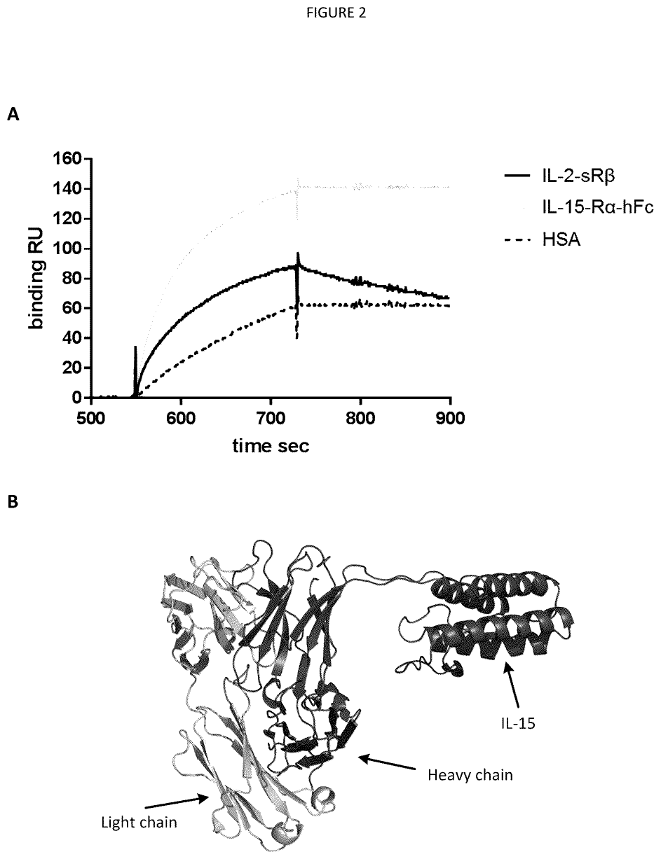

FIG. 2A shows the Biacore analysis of binding potency for this CA645/IL-15 antibody fusion protein when tested with 100 nM IL-2-Rp, IL-15-Ra (both of which are binding partners for IL-15) and 1000 nM HSA (ligand for CA645). As is clearly seen in FIG. 2A, binding was seen to all three ligands showing that functionality is retained in both the CA645 Fab portion as well as the IL-15 graft in the framework 3 region. FIG. 2B shows a modelled representation of the CA645/IL-15_{Fwk3} protein.

### Example 2—CA645 Fab with IL-2 Insert Polypeptide

Interleukin 2 (IL-2) is a pluripotent cytokine produced primarily by activated CD4+ T cells, which plays a crucial role in generating a normal immune response. IL-2 promotes proliferation and expansion of activated T lymphocytes, potentiates B cell growth, and activates monocytes and natural killer cells.

A fusion protein was generated and expressed in accordance with the above methods to insert the human IL-2 sequence into the framework 3 region of the CA645 Fab construct, as shown below.

Human IL-2 sequence:
(SEQ ID NO: 55)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT
CA645 Fab heavy chain with human IL-2 (bold and
italics) inserted into framework 3, linkers
underlined:
(SEQ ID NO: 56)
EVQLLESGGGLVQPGGSLRLSCAVSGIDLSNYAINWVRQAPGKGLEWIGI
IWASGTTFYATWAKGRFTISRDNSGGGSGGGGAPTSSSTKKTQLQLEHLL
LDLQMILNGINNYKNPKLTRMLTFKFYMPKKATELKHLQCLEEELKPLEE
VLNLAQSKNFHLRPRDLISNINVIVLELKGSETTFMCEYADETATIVEFL
NRWITFCQSIISTLTGGGKNTVYLQMNSLRAEDTAVYYCARTVPGYSTAP
YFDLWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSC

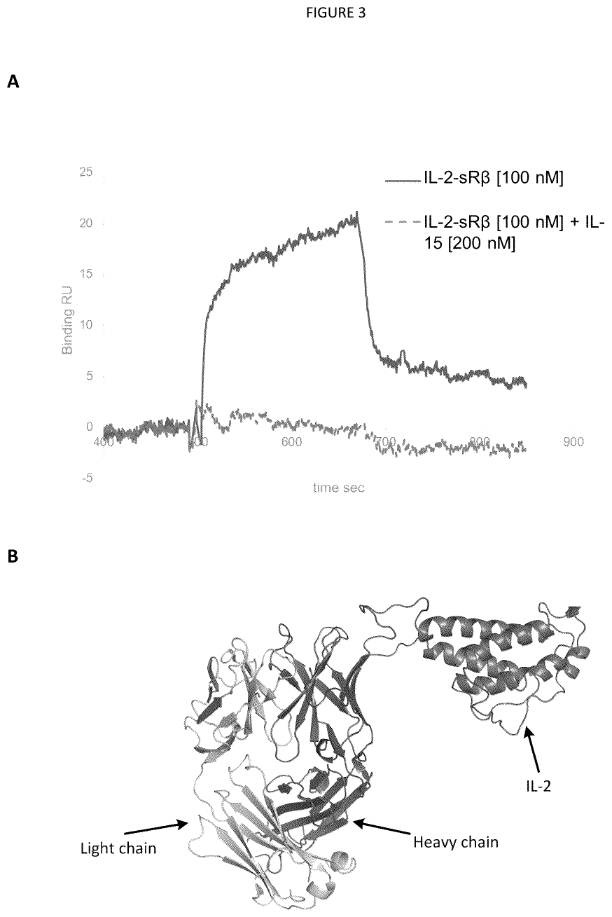

FIG. 3A shows the Biacore analysis of binding potency for this CA645/IL-2 antibody fusion protein when tested with 100 nM IL-2-Rβ (binding partner for IL-2), with and without prior incubation with 200 nM IL-15 (a competing ligand). As is shown in FIG. 3A, binding seen to IL-2-Rβ is abolished in the presence of the competing ligand in molar excess, showing that the binding seen to the IL-2 portion in framework 3 of the fusion protein is a genuine functional interaction and not non-specific. FIG. 3B shows a modelled representation of the CA645/IL-2_{Fwk3} protein.

### Example 3—CA645 Fab with G-CSF Insert Polypeptide

Granulocyte-colony stimulating factor (G-CSF) is a cytokine produced by a number of different cell types (e.g. endothelium, macrophages) which acts to simulate the production of mature ganulocytes from bone marrow.

A fusion protein was generated and expressed in accordance with the above methods to insert the human G-CSF sequence into the framework 3 region of the CA645 Fab construct, as shown below.

Human G-CSF sequence:
(SEQ ID NO: 57)
ATPLGPASSLPQSFLLKCLEQVRKIQGDGAALQEKLVSECATYKLCHPEE
LVLLGHSLGIPWAPLSSCPSQALQLAGCLSQLHSGLFLYQGLLQALEGIS
PELGPTLDTLQLDVADFATTIWQQMEELGMAPALQPTQGAMPAFASAFQR
RAGGVLVASHLQSFLEVSYRVLRHLAQP
CA645 Fab heavy chain with human G-CSF (bold and
italics) inserted into framework 3, Gly-Ser
linkers underlined:
(SEQ ID NO: 58)
EVQLLESGGGLVQPGGSLRLSCAVSGIDLSNYAINWVRQAPGKGLEWIGI
IWASGTTFYATWAKGRFTISRDNSGGGGSATPLGPASSLPQSFLLKCLEQ
VRKIQGDGAALQEKLVSECATYKLCHPEELVLLGHSLGIPWAPLSSCPSQ
ALQLAGCLSQLHSGLFLYQGLLQALEGISPELGPTLDTLQLDVADFATTI
WQQMEELGMAPALQPTQGAMPAFASAFQRRAGGVLVASHLQSFLEVSYRV
LRHLAQPSGGGGKNTVYLQMNSLRAEDTAVYYCARTVPGYSTAPYFDLWG
QGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSC

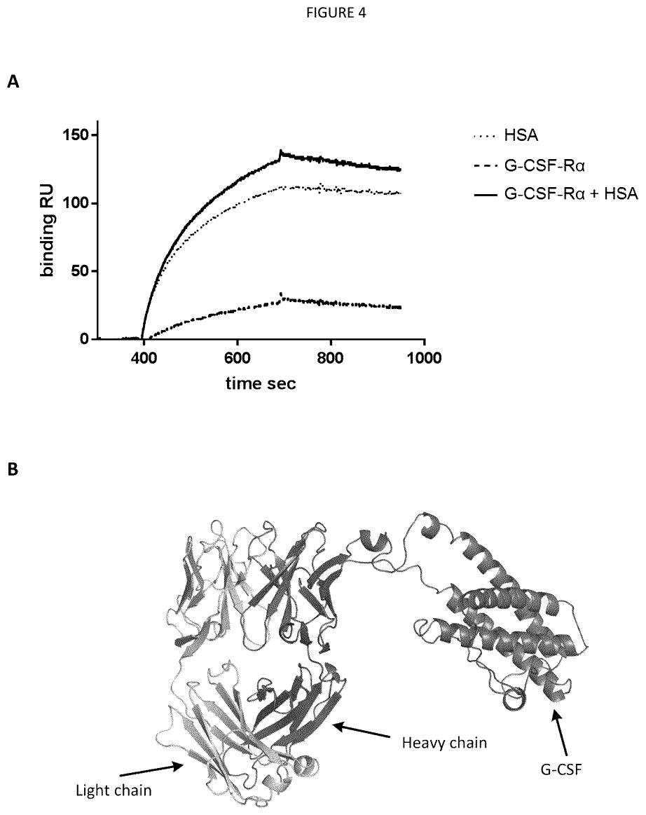

FIG. 4A shows the Biacore analysis of binding potency for this CA645/G-CSF antibody fusion protein when tested with 100 nM G-CSF-Rα (binding partner for G-CSF), 1000 nM HSA (ligand for CA645), and both G-CSF-Rα and HSA. FIG. 4A shows in line with the other Examples, both the G-CSF and Fab portions of the fusion protein are functionally active and are able to bind their respective binding partners. FIG. 4A further shows that in the presence of both binding partners, the signal seen from the SPR trace is additive from the single-binder traces, showing that binding to both functional portions can occur simultaneously. FIG. 4B shows a modelled representation of the CA645/G-CSF_{Fwk3} protein.

### Example 4—CA645 Fab with GM-CSF Insert Polypeptide

GM-CSF is a cytokine which stimulates stem cells to produce granulocytes (neutrophils, eosinophils, and basophils) and monocytes.

A fusion protein was generated and expressed in accordance with the above methods to insert the human and the murine GM-CSF sequences into the framework 3 region of the CA645 Fab construct, as shown below.

Human GM-CSF (hGM-CSF) sequence:
(SEQ ID NO: 59)
APARSPSPSTQPWEHVNAIQEARRLLNLSRDTAAEMNETVEVISEMFDLQ
REPTCLQTLELYKQGLRGSLTKLKGPLTMMASHYKQHCPPTPETSCATQI
ITFESFKENLKDFLLVIPFDCWEPVQE
CA645 Fab heavy chain with human GM-CSF (bold and
italics) inserted into framework 3, Gly-Ser linkers
underlined:
(SEQ ID NO: 60)
EVQLLESGGGLVQPGGSLRLSCAVSGIDLSNYAINWVRQAPGKGLEWIGI
IWASGTTFYATWAKGRFTISRDNSGGGSGGGGAPARSPSPSTQPWEHVNA
IQEARRLLNLSRDTAAEMNETVEVISEMFDLQEPTCLQTRLELYKQGLRG
SLTKLKGPLTMMASHYKQHCPPTPETSCATQIITFESFKENLKDFLLVIP
FDCWEPVQEGGGKNTVYLQMNSLRAEDTAVYYCARTVPGYSTAPYFDLWG
QGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSN
TKVDKKVEPKSC
CA645 Fab heavy chain with human GM-CSF (bold and
italics) at C-terminus of CH1, Gly-Ser linker
underlined (for comparison purposes):
(SEQ ID NO: 61)
EVQLLESGGGLVQPGGSLRLSCAVSGIDLSNYAINWVRQAPGKGLEWIGI
IWASGTTFYATWAKGRFTISRDNSKNTVYLQMNSLRAEDTAVYYCARTVP
GYSTAPYFDLWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCGGGSGGGGAPARSPSPSTQPWEHVNA
IQEARRLLNLSRDTAAEMNETVEVISEMFDLQEPTCLQTRLELYKQGLRG
SLTKLKGPLTMMASHYKQHCPPTPETSCATQIITFESFKENLKDFLLVIP
FDCWEPVQE
Murine GM-CSF (mGM-CSF) sequence:
(SEQ ID NO: 62)
APTRSPITVTRPWKHVEAIKEALNLLDDMPVTLNEEVEVVSNEFSFKKLT
CVQTRLKIFEQGLRGNFTKLKGALNMTASYYQTYCPPTPETDCETQVTTY
ADFIDSLKTFLTDIPFECKKPGQK
CA645 Fab heavy chain with murine GM-CSF (bold and
italics) inserted into framework 3, Gly-Ser linkers
underlined:
(SEQ ID NO: 63)
EVQLLESGGGLVQPGGSLRLSCAVSGIDLSNYAINWVRQAPGKGLEWIGI
IWASGTTFYATWAKGRFTISRDNSGGGSGGGGAPTRSPITVTRPWKHVEA
IKEALNLLDDMPVTLNEEVEVVSNEFSFKKLTCVQTRLKIFEQGLRGNFT
KLKGALNMTASYYQTYCPPTPETDCETQVTTYADFIDSLKTFLTDIPFEC
KKPGQKGGGKNTVYLQMNSLRAEDTAVYYCARTVPGYSTAPYFDLWGQGT
LVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKV
DKKVEPKSC
CA645 Fab heavy chain with murine GM-CSF (bold and
italics) at C-terminus of CH1, Gly-Ser linkers
underlined (for comparison purposes):
(SEQ ID NO: 64)
EVQLLESGGGLVQPGGSLRLSCAVSGIDLSNYAINWVRQAPGKGLEWIGI
IWASGTTFYATWAKGRFTISRDNSKNTVYLQMNSLRAEDTAVYYCARTVP
GYSTAPYFDLWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCGGGSGGGGAPTRSPITVTRPWKHVEA
IKEALNLLDDMPVTLNEEVEVVSNEFSFKKLTCVQTRLKIFEQGLRGNFT
KLKGALNMTASYYQTYCPPTPETDCETQVTTYADFIDSLKTFLTDIPFEC
KKPGQK

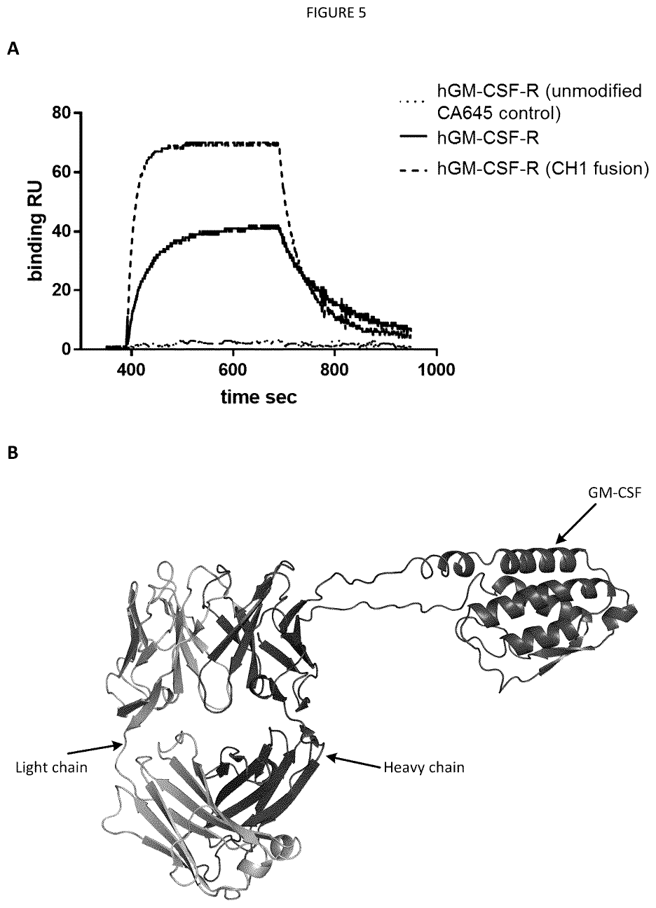

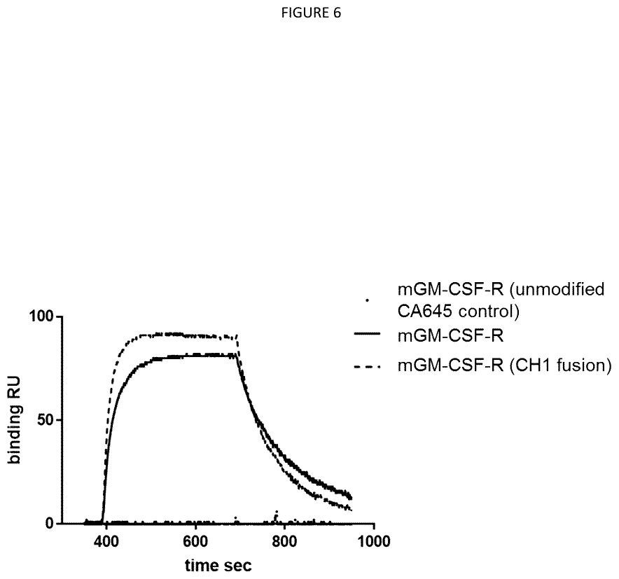

FIGS. 5A and 6 shows the Biacore analysis of binding potency for the CA645/hGM-CSF and CA645/mGM-CSF antibody fusion proteins (respectively) when tested with the cognate GM-CSF receptor (at 100 nM). The results demonstrate that the binding to the GM-CSF receptor is specific and dependent upon the presence of the insert polypeptide. The Figures also show a separate trace for a constant domain (CH1) fusion of the GM-CSF peptide, showing that the framework 3 insert functions in a comparable manner. FIG. 5B shows a modelled representation of the CA645/GM-CSFFwk3 protein.

### Example 5—CA645 Fab with EPO Insert Polypeptide

Erythropoietin (EPO) is a cytokine released by the kidney in response to cellular hypoxia which stimulates red blood cell production in the bone marrow.

A fusion protein was generated and expressed in accordance with the above methods to insert the human EPO sequence into the framework 3 region of the CA645 Fab construct, as shown below.

Human EPO sequence:
(SEQ ID NO: 65)
APPRLICDSRVLERYLLEAKEAENITTGCAEHCSLNENITVPDTKVNFYA
WKRMEVGQQAVEVWQGLALLSEAVLRGQALLVNSSQPWEPLQLHVDKAVS
GLRSLTTLLRALGAQKEAISPPDAASAAPLRTITADTFRKLFRVYSNFLR
GKLKLYTGEACRTGDR
CA645 Fab heavy chain with human EPO (bold and
italics) inserted into framework 3, Gly-Ser
linkers underlined:
(SEQ ID NO: 66)
EVQLLESGGGLVQPGGSLRLSCAVSGIDLSNYAINWVRQAPGKGLEWIGI
IWASGTTFYATWAKGRFTISRDNSGGGSGGGGAPPRLICDSRVLERYLLE
AKEAENITTGCAEHCSLNENITVPDTKVNFYAWKRMEVGQQAVEVWQGLA
LLSEAVLRGQALLVNSSQPWEPLQLHVDKAVSGLRSLTTLLRALGAQKEA
ISPPDAASAAPLRTITADTFRKLFRVYSNFLRGKLKLYTGEACRTGDRGG
GKNTVYLQMNSLRAEDTAVYYCARTVPGYSTAPYFDLWGQGTLVTVSSAS
TKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKS
C

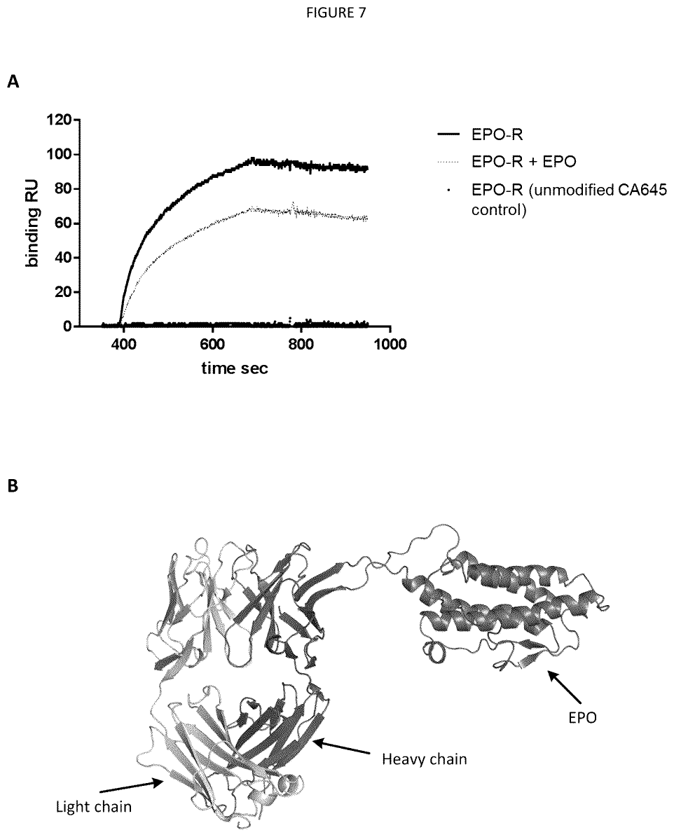

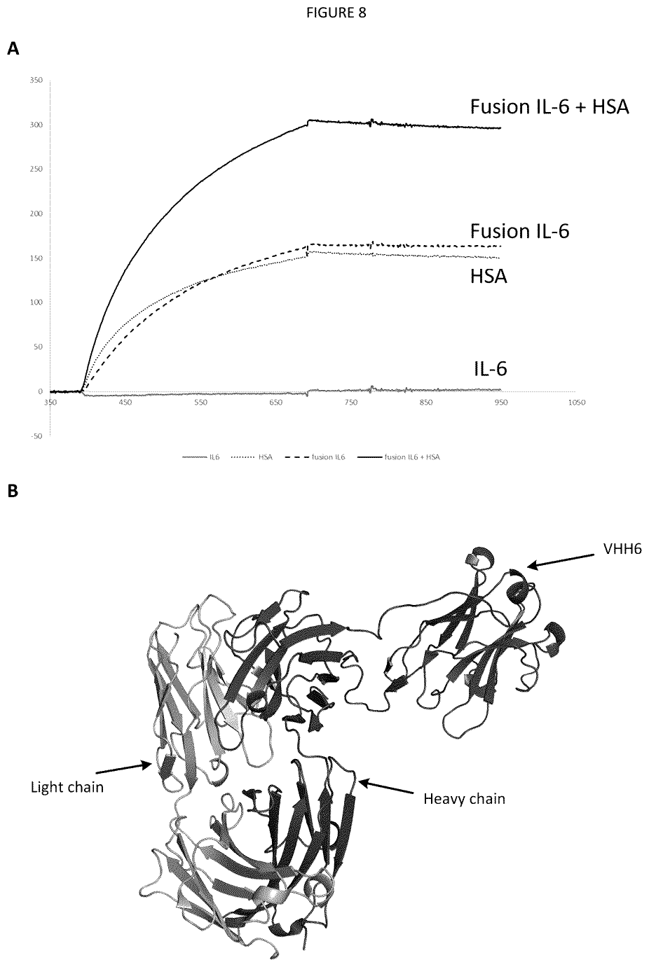

FIG. 7A shows the Biacore analysis of binding potency for this CA645/EPO antibody fusion protein when tested with 50 nM EPO-R (binding partner for EPO), with and without prior incubation with 500 nM soluble EPO at (competing ligand). FIG. 7A shows in line with the other Examples, the EPO portion of the fusion protein is functionally active and is able to bind its cognate receptor. Furthermore, the decrease in signal seen with the prior incubation of EPO shows the specificity of the interaction, while the unmodified CA645 Fab acts as a further control to demonstrate that the binding seen is derived from the EPO graft. FIG. 7B shows a modelled representation of the CA645/EPO_{Fwk3} protein.

### Example 6—Antibody/Fab Fusion Protein with CA645 Fab with VHH6 Insert

VHH6 is an engineered single-domain heavy-chain camelid antibody which binds a junctional epitope in that it is able to bind the IL-6:gp80 complex, but not the individual components alone.

A fusion protein was generated and expressed in accordance with the above methods to insert the VHH6 sequence into the framework 3 region of the CA645 Fab construct, as shown below.

Camelid VHH6 sequence:
(SEQ ID NO: 67)
DVQFVESGGGSVHAGGSLRLNCATSGYIYSTYCMGWFRQAPGKEREGVAH
IYTNSGRTYYADSVKGRFTISQDNAKNTVYLQMNSLKPEDTAIYYCAARP
SIRCASFSATEYKDWGQGTQVTVSS
CA645 Fab heavy chain with VHH6 (bold and italics)
inserted into framework 3, Gly-Ser linkers
underlined:
(SEQ ID NO: 68)
EVQLLESGGGLVQPGGSLRLSCAVSGIDLSNYAINWVRQAPGKGLEWIGI
IWASGTTFYATWAKGRFTISRDNSGGGGSGGGGSDVQFVESGGGSVHAGG
SLRLNCATSGYIYSTYCMGWFRQAPGKEREGVAHIYTNSGRTYYADSVKG
RFTISQDNAKNTVYLQMNSLKPEDTAIYYCAARPSIRCASFSATEYKDWG
QGTQVTVSSGGGGSGGGGSKNTVYLQMNSLRAEDTAVYYCARTVPGYSTA
PYFDLWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSC

FIG. 8A shows the Biacore analysis of binding potency for this CA645/VHH6 antibody fusion protein when tested with 100 nM IL-6 (negative control for VHH6 as VHH6 solely binds the complex), 100 nM IL-6/gp80 fusion (binding partner for VHH6), 1000 nM HSA (binding partner for CA645) and both the IL-6 fusion and HSA together. FIG. 8A shows in line with the other Examples, both the VHH6 and CA645 parts of the antibody fusion protein are functionally active as binding fragments and are able to bind their respective binding partners. FIG. 8A further shows that in the presence of both binding partners, the signal seen from the SPR trace is additive from the single-binder traces, showing that binding to both functional portions can occur simultaneously. FIG. 8B shows a modelled representation of the CA645/VHH6Fwk3 protein.

### Example 7—Antibody/Fab Fusion Protein with CA645 Fab with VHH15 Insert

VHH15 is an engineered single domain heavy-chain camelid antibody which recognises and binds to IL-6.

A fusion protein was generated and expressed in accordance with the above methods to insert the VHH15 sequence into the framework 3 region of the CA645 Fab construct, as shown below.

Camelid VHH15 sequence:
(SEQ ID NO: 69)
QVQLVESGGGSVQAGGSLRLSCVAASGYTGCTYDMRWYRQAPGKEREFVS
GIDSDGRATYADSVKGRFTISQSNAKIAVYLQMDSLKLEDTAMYYCNLQC
LRYPGEYYWGQGTQVTVSS
CA645 Fab heavy chain with VHH15, an anti-IL-6
camelid domain antibody (bold and italics),
inserted into framework 3, Gly-Ser linkers
underlined:
(SEQ ID NO: 70)
EVQLLESGGGLVQPGGSLRLSCAVSGIDLSNYAINWVRQAPGKGLEWIGI
IWASGTTFYATWAKGRFTISRDNSGGGGSGGGGSQVQLVESGGGSVQAGG
SLRLSCVAASGYTGCTYDMRWYRQAPGKEREFVSGIDSDGRATYADSVKG
RFTISQSNAKIAVYLQMDSLKLEDTAMYYCNLQCLRYPGEYYWGQGTQVT
VSSGGGGSGGGGSKNTVYLQMNSLRAEDTAVYYCARTVPGYSTAPYFDLW
GQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPS
NTKVDKKVEPKSC

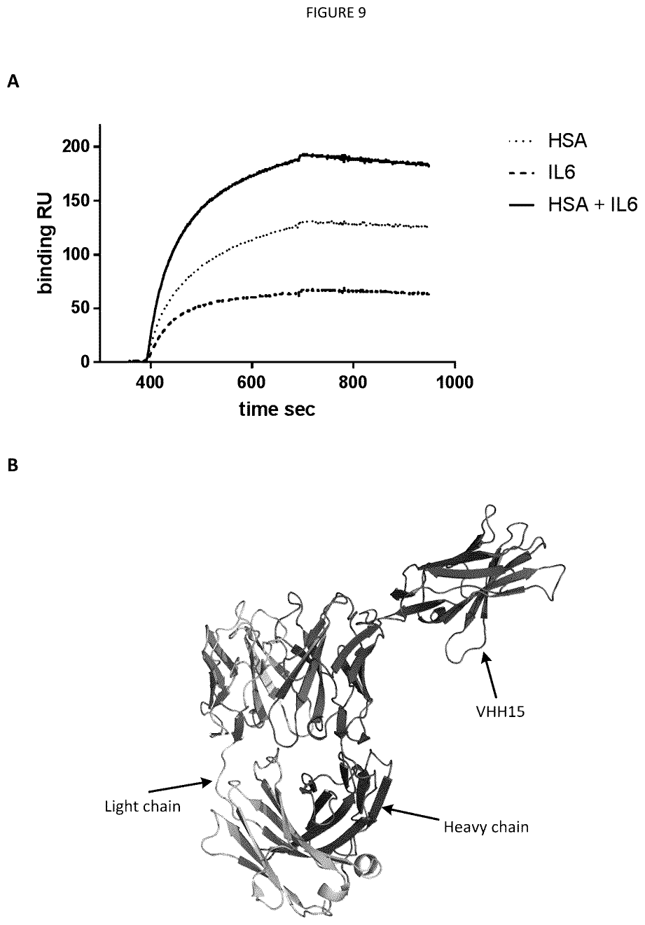

FIG. 9A shows the Biacore analysis of binding potency for this CA645/VHH15 antibody fusion protein when tested with 100 nM IL-6 (ligand for VHH15), 1000 nM HSA (ligand for CA645), and both IL-6 and HSA. FIG. 9A shows in line with the other Examples, both the VHH15 and Fab portions of the antibody fusion protein are functionally active as binding fragments and are able to bind their respective binding partners. FIG. 9A further shows that in the presence of both binding partners, the signal seen from the SPR trace is additive from the single-binder traces, showing that binding to both functional portions can occur simultaneously. FIG. 9B shows a modelled representation of the CA645/VHH15_{Fwk3} protein.

### Example 8—CA645 Fab with Sclerostin Insert Polypeptide

Sclerostin is the product of the SOST gene in humans and is involved in the process of bone formation, being largely expressed in osteocytes. Sclerostin functions as a regulatory protein in the Wnt signalling pathway through binding to the LRP-type co-receptors.

Fusion proteins were generated and expressed in accordance with the above methods to insert the core or the whole Sclerostin sequence into the framework 3 region of the CA645 Fab construct, as shown below.

Human mature full-length sclerostin sequence:
(SEQ ID NO: 71)
QGWQAFKNDATEIIPELGEYPEPPPELENNKTMNRAENGGRPPHHPFETK
DVSEYSCRELHFTRYVTDGPCRSAKPVTELVCSGQCGPARLLPNAIGRGK
WWRPSGPDFRCIPDRYRAQRVQLLCPGGEAPRARKVRLVASCKCKRLTRF
HNQSELKDFGTEAARPQKGRKPRPRARSAKANQAELENAY
Sequence of core domain of human sclerostin:
(SEQ ID NO: 72)
CRELHFTRYVTDGPCRSAKPVTELVCSGQCGPARLLPNAIGRGKWWRPSG
PDFRCIPDRYRAQRVQLLCPGGEAPRARKVRLVASCKC
CA645 Fab heavy chain with human sclerostin core
domain (bold and italics) inserted into framework
3, single S linkers underlined:
(SEQ ID NO: 73)
EVQLLESGGGLVQPGGSLRLSCAVSGIDLSNYAINWVRQAPGKGLEWIGI
IWASGTTFYATWAKGRFTISRDNSSCRELHFTRYVTDGPCRSAKPVTELV
CSGQCGPARLLPNAIGRGKWWRPSGPDFRCIPDRYRAQRVQLLCPGGEAP
RARKVRLVASCKCSKNTVYLQMNSLRAEDTAVYYCARTVPGYSTAPYFDL
WGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDKKVEPKSC
CA645 Fab heavy chain with human sclerostin core
domain (bold and italics) inserted into framework
3, Gly-Ser (2X G₄S) linkers underlined:
(SEQ ID NO: 74)
EVQLLESGGGLVQPGGSLRLSCAVSGIDLSNYAINWVRQAPGKGLEWIGI
IWASGTTFYATWAKGRFTISRDNSGGGGSGGGGSCRELHFTRYVTDGPCR
SAKPVTELVCSGQCGPARLLPNAIGRGKWWRPSGPDFRCIPDRYRAQRVQ
LLCPGGEAPRARKVRLVASCKCSGGGGSGGGGSKNTVYLQMNSLRAEDTA
VYYCARTVPGYSTAPYFDLWGQGTLVTVSSASTKGPSVFPLAPSSKSTSG
GTAALGCLVKDYFPEPVTVSWNSGALTSGVHTFPAVLQSSGLYSLSSVVT
VPSSSLGTQTYICNVNHKPSNTKVDKKVEPKSC
CA645 Fab heavy chain with human sclerostin core
domain (bold and italics) inserted into framework
3, rigid linkers underlined:
(SEQ ID NO: 75)
EVQLLESGGGLVQPGGSLRLSCAVSGIDLSNYAINWVRQAPGKGLEWIGI
IWASGTTFYATWAKGRFTISRDNSIPFTVCRELHFTRYVTDGPCRSAKPV
TELVCSGQCGPARLLPNAIGRGKWWRPSGPDFRCIPDRYRAQRVQLLCPG
GEAPRARKVRLVASCKCEYHGLQKNTVYLQMNSLRAEDTAVYYCARTVPG
YSTAPYFDLWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSC
CA645 Fab heavy chain with human mature full-
length sclerostin (bold and italics) inserted into
framework 3, Gly-Ser (1X G₄S) linkers underlined:
(SEQ ID NO: 76)
EVQLLESGGGLVQPGGSLRLSCAVSGIDLSNYAINWVRQAPGKGLEWIGI
IWASGTTFYATWAKGRFTISRDNSGGGGSQGWQAFKNDATEIIPELGEYP
EPPPELENNKTMNRAENGGRPPHHPFETKDVSEYSCRELHFTRYVTDGPC
RSAKPVTELVCSGQCGPARLLPNAIGRGKWWRPSGPDFRCIPDRYRAQRV
QLLCPGGEAPRARKVRLVASCKCKRLTRFHNQSELKDFGTEAARPQKGRK
PRPRARSAKANQAELENAYSGGGGSKNTVYLQMNSLRAEDTAVYYCARTV
PGYSTAPYFDLWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSC

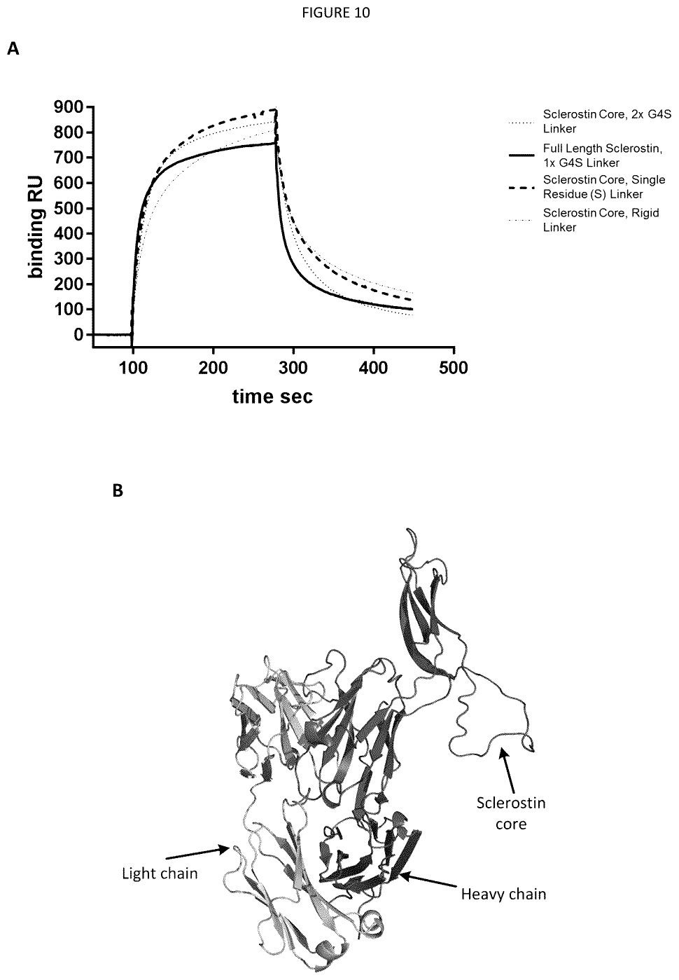

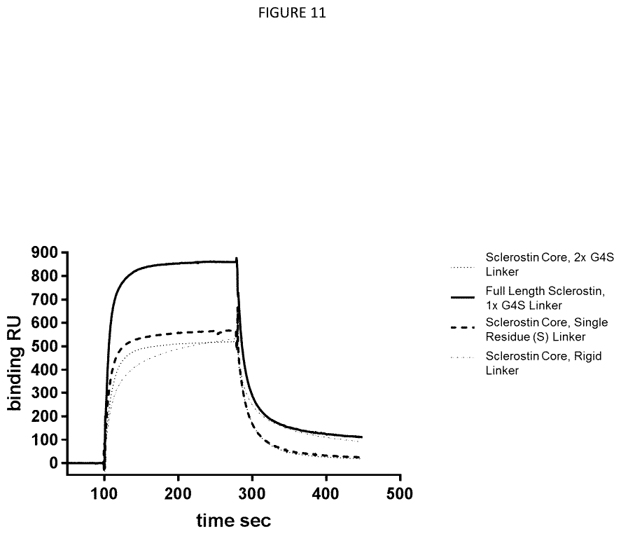

FIGS. 10A and 11 show the Biacore analysis of potency for this CA645/sclerostin antibody fusion protein when tested as 1 in 5 diluted supernatant for binding to immobilized extracellular domains of LRP6 and LRP4 respectively (binding partners for Sclerostin). This experiment also investigated the effects of different linker region constructs. It was noted that all of the tested constructs showed binding to LRP6 and LRP4. FIG. 10B shows a modelled representation of the CA645/Sclerostin_{core-Fwk3} protein.

### Example 9—CA645 Fab with IL-10 Insert Polypeptide (IL-10 Dimer)

Interleukin 10 (IL-10), also known as human cytokine synthesis inhibitory factor (CSIF), is an anti-inflammatory cytokine. It functions generally as a homodimer.

A fusion protein was generated and expressed in accordance with the above methods to insert two human IL-10 sequences into the framework 3 region of the CA645 Fab construct, as shown below.

Human IL-10 monomer sequence:
(SEQ ID NO: 100)
NCWLRQAKNGRCQVLYKTELSKEECCSTGRLSTSWTEEDVNDNTLFKWMI
FNGGAPNCIPCKEPRCVCAPDCSNITWKGPVCGLDGKTYRNECALLKARC
KEQPELEVQYQGRCKKTCRDVFCPGSSTCVVDQTNNAYCVTCNRICPEPA
SSEQYLCGNDGVTYSSACHLRKATCLLGRSIGLAGEYK
CA645 Fab heavy chain with two human IL-10 (bold
and italics) monomers inserted into framework 3,
linkers underlined:
(SEQ ID NO: 101)
EVQLLESGGGLVQPGGSLRLSCAVSGIDLSNYAINWVRQAPGKGLEWIGI
IWASGTTFYATWAKGRFTISRDNSGGGGSGSNCWLRQAKNGRCQVLYKTE
LSKEECCSTGRLSTSWTEEDVNDNTLFKWMIFNGGAPNCIPCKEPRCVCA
PDCSNITWKGPVCGLDGKTYRNECALLKARCKEQPELEVQYQGRCKKTCR
DVFCPGSSTCVVDQTNNAYCVTCNRICPEPASSEQYLCGNDGVTYSSACH
LRKATCLLGRSIGLAGEYKGGGGSGGGGSGGGGSNCWLRQAKNGRCQVLY
KTELSKEECCSTGRLSTSWTEEDVNDNTLFKWMIFNGGAPNCIPCKEPRC
VCAPDCSNITWKGPVCGLDGKTYRNECALLKARCKEQPELEVQYQGRCKK
TCRDVFCPGSSTCVVDQTNNAYCVTCNRICPEPASSEQYLCGNDGVTYSS
ACHLRKATCLLGRSIGLAYEGGGGSGSKNTVYLQMNSLRAEDTAVYYCAR
TVPGYSTAPYFDLWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSC

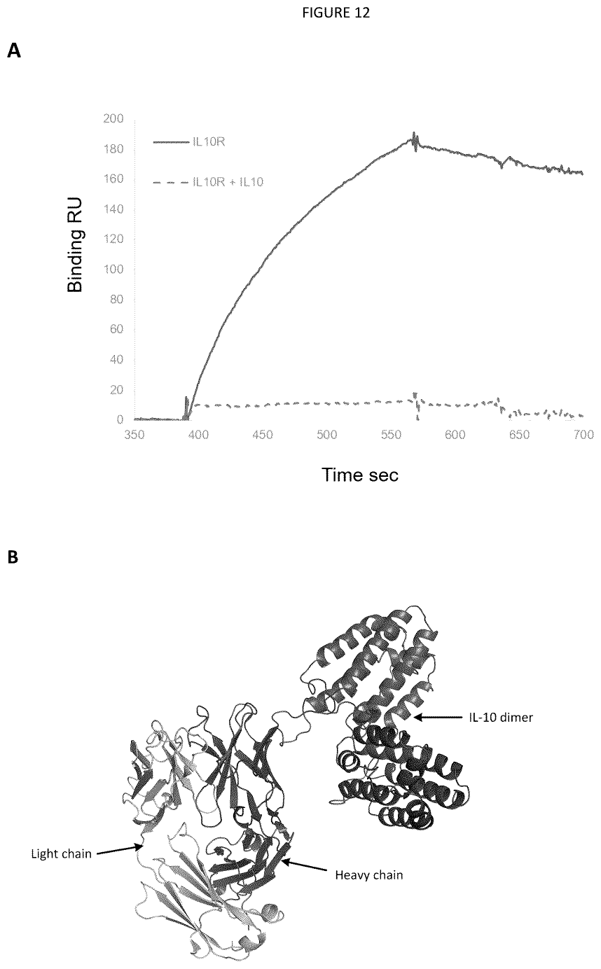

FIG. 12A shows the Biacore analysis of binding potency for this CA645/IL-10 antibody fusion protein when tested with 100 nM IL-10 receptor (binding partner for IL-10), with and without prior incubation with 200 nM IL-10 (competing ligand). As is shown in FIG. 12A, binding seen to IL-10 receptor is abolished in the presence of the competing ligand in molar excess, showing that the binding seen to the IL-10 portion in framework 3 of the fusion protein is a genuine functional interaction and not non-specific. This result also suggests that the inserted IL-10 monomers are also able to form a functional dimer within the framework 3 construct. FIG. 12B shows a modelled representation of the CA645/IL-10Fwk3 protein.

### Example 10—Antibody-Framework 3 Insertion Constructs with Other Fabs

Fusion proteins were generated and expressed in accordance with the above methods to insert the human IL-15 sequence into the framework 3 region of the CA497 and CA240 Fab constructs, as shown below.

Anti-IL-17A CA497 Fab heavy chain sequence (Fab,
CH1 from IgG1; Variable domain underlined; CDRs
in bold and italics.)
(SEQ ID NO: 125)
EVQLVESGGGLVKPGGSLRLSCAASGVIFSDYYMAWVRQAPGKGLEWVA
SINFNADISYYRESVKGRFTISRDDSKNTLYLQMNSLKTEDTAVYYCTT
DANRQNYDWFAYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSC
Anti-IL-17A CA497 Fab heavy chain with human
IL-15 (bold and italics) inserted into framework
3, Gly-Ser linkers underlined:
(SEQ ID NO: 83)
EVQLVESGGGLVKPGGSLRLSCAASGVIFSDYYMAWVRQAPGKGLEWVA
SINFNADISYYRESVKGRFTISRDDSGGGSGGGGNWVNVISDLKKIEDL
IQSMHIDATLYTESDVHPSCKVTAMKCFLLELQVISLESGDASIHDTVE
NLIILANNSLSSNGNVTESGCKECEELEEKNIKEFLQSFVHIVQMFING
GGKNTLYLQMNSLKTEDTAVYYCTTDANRQNYDWFAYWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSC
Anti-IL-17A CA497 Fab light chain sequence:
(SEQ ID NO: 84)
AIQLTQSPSSLSASVGDRVTITCKASESVSSSMYSYMHWYQQKPGKAPK
LLIYRASNLESGVPSRFSGSGSGTDFTLTISSLQPEDFATYYCQQSWTA
PRTFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC
Anti-IL-6 CA240 Fab heavy chain sequence (Fab,
CH1 from IgG1; Variable domain underlined;
CDRs in bold and italics):
(SEQ ID NO: 126)
EVQLVESGGGLVQPGGSLRLSCAASGFNFNDYFMNWVRQAPGKGLEWVA
QMRNKNYQYGTYYAESLEGRFTISRDDSKNSLYLQMNSLKTEDTAVYYC
ARESYYGFTSYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSC
Anti-IL-6 CA240 Fab heavy chain with human IL-15
(bold and italics) inserted into framework 3,
Gly-Ser linkers underlined:
(SEQ ID NO: 85)
EVQLVESGGGLVQPGGSLRLSCAASGFNFNDYFMNWVRQAPGKGLEWVA
QMRNKNYQYGTYYAESLEGRFTISRDDSGGGSGGGGNWVNVISDLKKIE
DLIQSMHIDATLYTESDVHPSCKVTAMKCFLLELQVISLESGDASIHDT
VENLIILANNSLSSNGNVTESGCKECEELEEKNIKEFLQSFVHIVQMFI
NGGGKNSLYLQMNSLKTEDTAVYYCARESYYGFTSYWGQGTLVTVSSAS
TKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEP
KSC
Anti-IL-6 CA240 Fab light chain sequence:
(SEQ ID NO: 86)
DIQMTQSPSSLSASVGDRVTITCQASQDIGISLSWYQQKPGKAPKLLIY
NANNLADGVPSRFSGSGSGTDFTLTISSLQPEDFATYYCLQHNSAPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

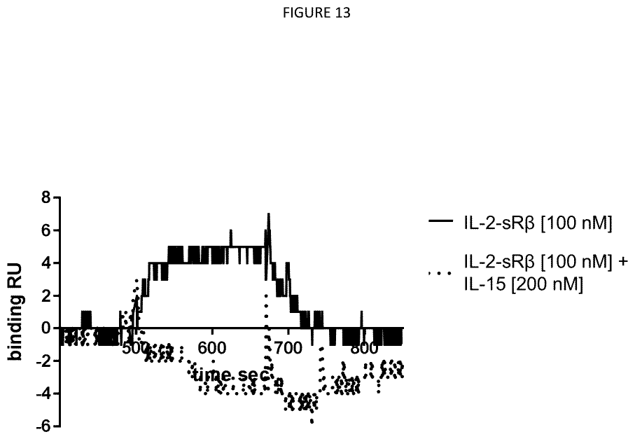

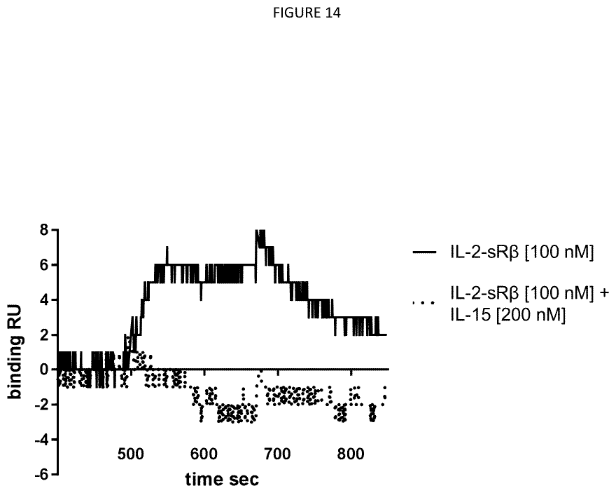

FIGS. 13 and 14 show the Biacore analysis of binding potency for the CA497/IL-15 and CA240/IL-15 (respectively) antibody fusion proteins when tested with 100 nM IL-2-Rβ (binding partner for IL-15), with and without prior incubation with IL-15 (a competing ligand). As is shown in both experiments, binding seen to IL-2-Rβ is abolished in the presence of the competing ligand in molar excess, showing that the binding seen to the IL-15 portion in framework 3 of the fusion proteins is a genuine functional interaction and not non-specific.

### Example 11—Fab Constructs with a VHH Inserted into Framework 3

HyHEL5 heavy chain:
(SEQ ID NO: 87)
EVQLQQSGAELMKPGASVKISCKASGYTFSDYWIEWVKQRPGHGLEWIG
EILPGSGSTNYHERFKGKATFTADTSSSTAYMQLNSLTSEDSGVYYCLH
GNYDFDGWGQGTTLTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSC
HyHEL5 light chain:
(SEQ ID NO: 88)
DIVLTQSPAIMSASPGEKVTMTCSASSSVNYMYWYQQKSGTSPKRWIYD
TSKLASGVPVRFSGSGSGTSYSLTISSMETEDAATYYCQQWGRNPTFGG
GTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTH
QGLSSPVTKSFNRGEC
D3L11:
(SEQ ID NO: 89)
DVQLVESGGGSVQAGGSLRLSCAASGSTDSIEYMTWFRQAPGKAREGVA
ALYTHTGNTYYTDSVKGRFTISQDKAKNMAYLRMDSVKSEDTAIYTCGA
TRKYVPVRFALDQSSYDYWGQGTQVTVSS

The following fusion constructs were generated to test whether inserting a second antibody molecule into the framework 3 region of a first antibody where the antibodies bound the same target antigen would result in a greater affinity for the antigen than either of the individual antibodies.

Anti-lysozyme HyHEL5 light chain with anti-
lysozyme VHH D3L11 (bold) inserted into framework
3, Gly-Ser linkers underlined:
(SEQ ID NO: 90)
DIVLTQSPAIMSASPGEKVTMTCSASSSVNYMYWYQQKSGTSPKRWIYD
TSKLASGVPVRFSGSGSGSGGGGSDVQLVESGGGSVQAGGSLRLSCAAS
GSTDSIEYMTWFRQAPGKAREGVAALYTHTGNTYYTDSVKGRFTISQDK
AKNMAYLRMDSVKSEDTAIYTCGATRKYVPVRFALDQSSYDYWGQGTQV
TVSSGGGGSGGGGSGGGGSTSYSLTISSMETEDAATYYCQQWGRNPTFG
GGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
Anti-lysozyme HyHEL5 light chain with anti-IL-6
VHH15 (bold) inserted into framework 3, Gly-Ser
linkers underlined:
(SEQ ID NO: 91)
DIVLTQSPAIMSASPGEKVTMTCSASSSVNYMYWYQQKSGTSPKRWIYD
TSKLASGVPVRFSGSGSGSGGGGSQVQLVESGGGSVQAGGSLRLSCVAA
SGYTGCTYDMRWYRQAPGKEREFVSGIDSDGRATYADSVKGRFTISQSN
AKIAVYLQMDSLKLEDTAMYYCNLQCLRYPGEYYWGQGTQVTVSSGGGG
SGGGGSGGGGSTSYSLTISSMETEDAATYYCQQWGRNPTFGGGTKLEIK
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC
CA645 light chain with anti-lysozyme VHH D3L11
(bold) inserted into framework 3, Gly-Ser linkers
underlined:
(SEQ ID NO: 92)
DIQMTQSPSSVSASVGDRVTITCQSSPSVWSNFLSWYQQKPGKAPKLLI
YEASKLTSGVPSRFSGSGSGSGGGGSDVQLVESGGGSVQAGGSLRLSCA
ASGSTDSIEYMTWFRQAPGKAREGVAALYTHTGNTYYTDSVKGRFTISQ
DKAKNMAYLRMDSVKSEDTAIYTCGATRKYVPVRFALDQSSYDYWGQGT
QVTVSSGGGGSGGGGSGGGGSTDFTLTISSLQPEDFATYYCGGGYSSIS
DTTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

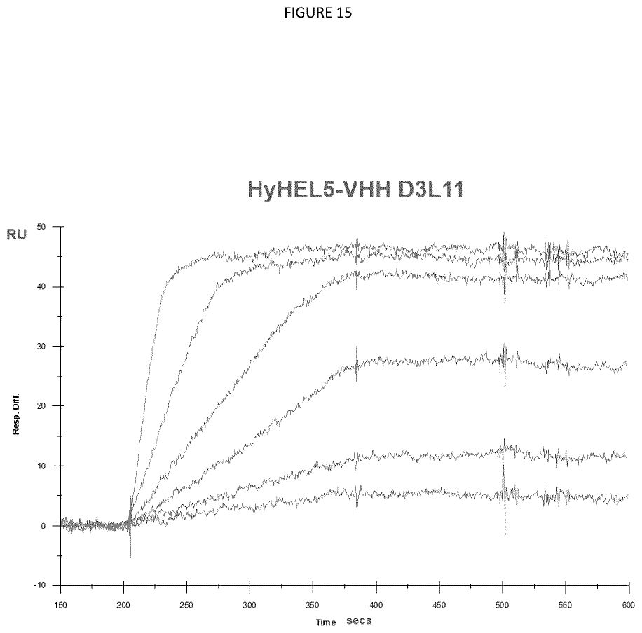

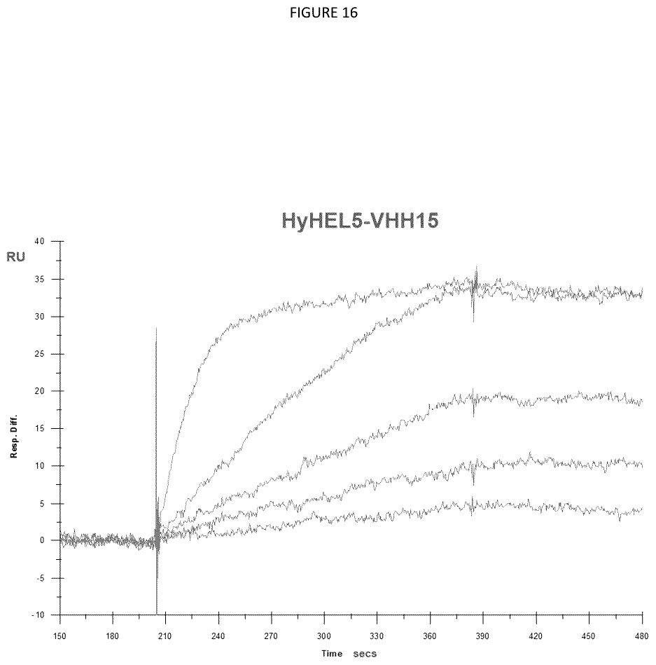

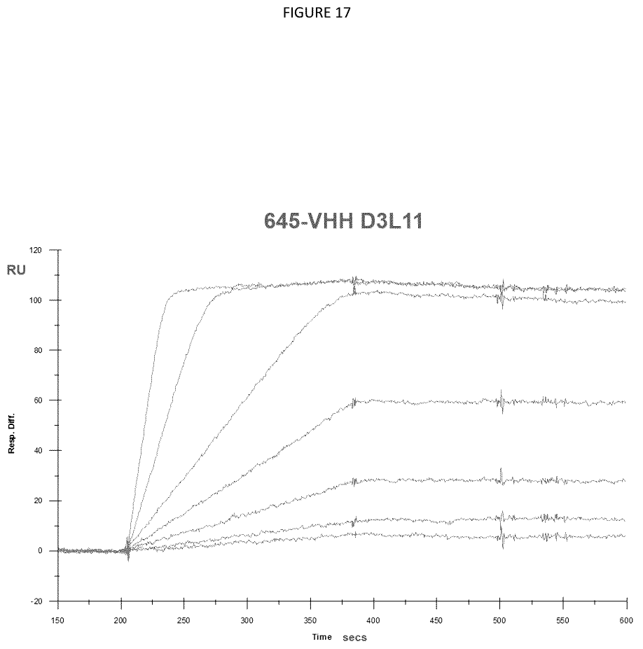

The SPR traces from the dilution series conducted for each of the constructs when tested for binding to lysozyme is shown in FIGS. 15 to 17. From the subsequent analysis, using the methods described above, the following table reveals the binding affinity for lysozyme calculated for each construct:

TABLE 2
Binding affinities for antibody constructs

| | | | Binding |
|---|---|---|---|
| | | | Affinity for |
| | Fab | VHH | lysozyme |
| construct | specificity | specificity | (K_{D}) |
| Antibody: HyHEL5 Fab | lysozyme | lysozyme | 2.6 × 10⁻¹¹M |
| Framework 3 Insert: VHH D3L11 | | | |
| Antibody: HyHEL5 Fab | lysozyme | IL-6 | 3.1 × 10⁻¹⁰M |
| Framework 3 Insert: VHH15 | | | |
| Antibody: CA645 Fab | Human | lysozyme | 4.8 × 10⁻¹⁰M |
| Framework 3 Insert: VHH D3L11 | serum | | |
| | albumin | | |

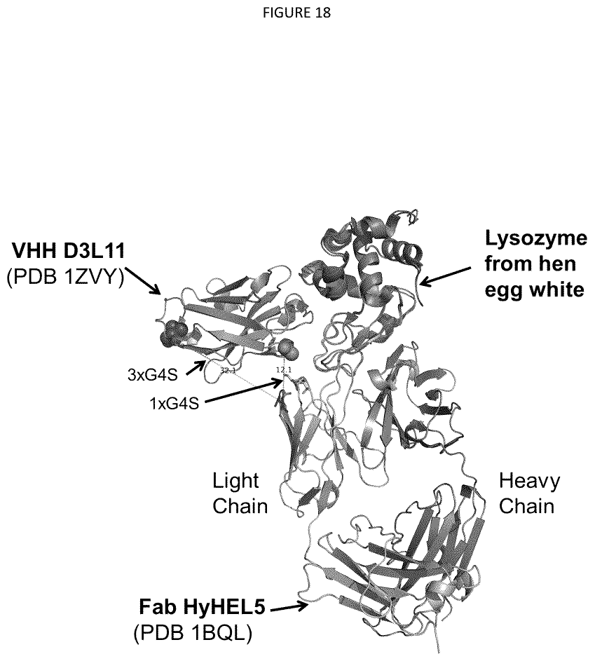

As is clearly seen from the above results, the binding affinity for lysozyme increases (i.e. a decrease is seen in the K_{D}) for the construct where the Fab and the VHH insert both bound to lysozyme. FIG. 18 illustrates how this may happen at a molecular level. This also hypothesises that the linker length may be important to enable multivalent interactions on the same target molecule in such antibodies.

### Example 12—CA645 Fab with IL-15 Insert Polypeptide Using Bovine Linkers

Variants were generated on the CA645/IL-15Fwk3 protein described in Example 1, where the linkers were changed to other types of linkers including bovine linkers, and their effects on binding tested. As well as the original heavy chain construct (SEQ ID NO: 54), further constructs were generated as shown below:

CA645 Fab heavy chain with human IL-15 (bold and
italics) inserted into framework 3 with bovine
long linker (underlined)
(SEQ ID NO: 93)
EVQLLESGGGLVQPGGSLRLSCAVSGIDLSNYAINWVRQAPGKGLEWIGI
IWASGTTFYATWAKGRFTISRDNSTSVHQETKKYQSNWVNVISDLKKIED
LIQSMHIDATLYTESDVHPSCKVTAMKCFLLELQVISLESGDASIHDTVE
NLIILANNSLSSNGNVTESGCKECEELEEKNIKEFLQSFVHIVQMFINSY
TYNYEWHVDVKNTVYLQMNSLRAEDTAVYYCARTVPGYSTAPYFDLWGQG
TLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTK
VDKKVEPKSC
CA645 Fab heavy chain with human IL-15 (bold and
italics) inserted into framework 3 with bovine
short linker (underlined)
(SEQ ID NO: 94)
EVQLLESGGGLVQPGGSLRLSCAVSGIDLSNYAINWVRQAPGKGLEWIGI
IWASGTTFYATWAKGRFTISRDNSETKKYQSNWVNVISDLKKIEDLIQSM
HIDATLYTESDVHPSCKVTAMKCFLLELQVISLESGDASIHDTVENLIIL
ANNSLSSNGNVTESGCKECEELEEKNIKEFLQSFVHIVQMFINSYTYNYE
KNTVYLQMNSLRAEDTAVYYCARTVPGYSTAPYFDLWGQGTLVTVSSAST
KGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSC
CA645 Fab heavy chain with human IL-15 (bold and
italics) inserted into framework 3 with 2xG₄S
linker (underlined)
(SEQ ID NO: 95)
EVQLLESGGGLVQPGGSLRLSCAVSGIDLSNYAINWVRQAPGKGLEWIGI
IWASGTTFYATWAKGRFTISRDNSGGGGSGGGGSNWVNVISDLKKIEDLI
QSMHIDATLYTESDVHPSCKVTAMKCFLLELQVISLESGDASIHDTVENL
IILANNSLSSNGNVTESGCKECEELEEKNIKEFLQSFVHIVQMFINGGGG
SGGGGSKNTVYLQMNSLRAEDTAVYYCARTVPGYSTAPYFDLWGQGTLVT
VSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKK
VEPKSC

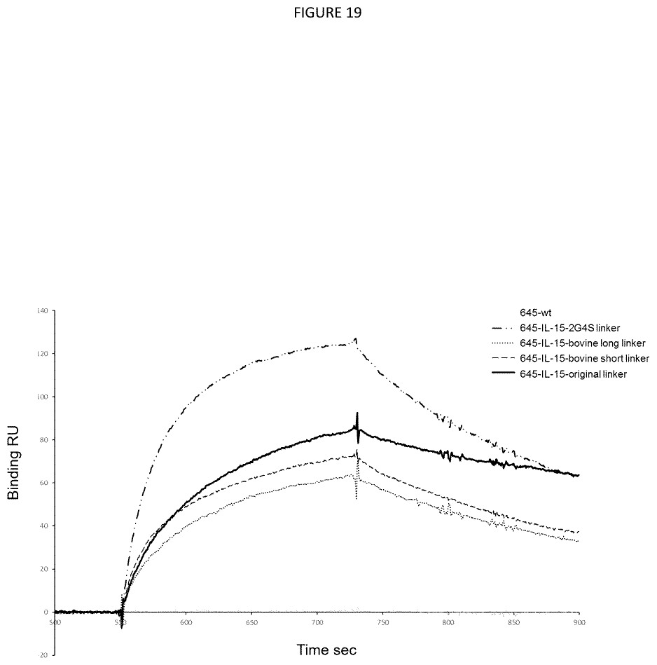

FIG. 19 shows the Biacore binding analysis of the above CA645/IL-15Fwk3 constructs when tested with 100 nM IL-2-Rβ, and compared against the construct in Example 1 (original linker) and the wild type CA645 Fab, which has no IL-15 insert. It was noted that all of the tested constructs showed successful binding.

### Example 13—CA645 Fab Fragment with a dsscFv Inserted into Framework 3

### Gene Design

Two antibodies of the invention were generated and expressed in accordance with the above methods to insert an anti-IL17A CA497 dsscFv (vHvL, or vLvH) into the framework 3 region of the CA645 Fab fragment (scaffold antibody), as shown below. The resulting antibodies may be termed CA645 Fab-Fwk3 CA497 dsscFv (vHvL) or CA645 Fab-Fwk3 CA497 dsscFv (vLvH) respectively. The CA497 dsscFv and the CA645 Fab were generated as controls. Genes encoding light and heavy chain V-regions, including variants with a framework 3 insertion, were designed and constructed by an automated synthesis approach (ATUM). Light chain V-region genes (e.g, coding for the CA645 VL domain of SEQ ID NO: 2) were cloned into expression vectors containing DNA encoding human Cx region (Km3 allotype). Heavy chain V-region genes were cloned into expression vectors containing DNA encoding IgG1 CH1 region.

CA497 dsscFv (vHvL)
(SEQ ID NO: 127)
EVQLVESGGGLVKPGGSLRLSCAASGVIFSDYYMAWVRQAPGKCLEWVAS
INFNADISYYRESVKGRFTISRDDSKNTLYLQMNSLKTEDTAVYYCTTDA
NRQNYDWFAYWGQGTLVTVSSGGGGSGGGGSGGGGSAIQLTQSPSSLSAS
VGDRVTITCKASESVSSSMYSYMHWYQQKPGKAPKLLIYRASNLESGVPS
RFSGSGSGTDFTLTISSLQPEDFATYYCQQSWTAPRTFGCGTKVEIK
CA497 dsscFv (vLvH)
(SEQ ID NO: 128)
AIQLTQSPSSLSASVGDRVTITCKASESVSSSMYSYMHWYQQKPGKAPKL
LIYRASNLESGVPSRFSGSGSGTDFTLTISSLQPEDFATYYCQQSWTAPR
TFGCGTKVEIKGGGGSGGGGSGGGGSEVQLVESGGGLVKPGGSLRLSCAA
SGVIFSDYYMAWVRQAPGKCLEWVASINFNADISYYRESVKGRFTISRDD
SKNTLYLQMNSLKTEDTAVYYCTTDANRQNYDWFAYWGQGTLVTVSS
CA645 Fab heavy chain with CA497 dsscFv (vHvL)
(bold and italics) inserted into framework 3,
linkers underlined
(SEQ ID NO: 129)
EVQLLESGGGLVQPGGSLRLSCAVSGIDLSNYAINWVRQAPGKGLEWIGI
IWASGTTFYATWAKGRFTISRDNSGGGGSGGGGSEVQLVESGGGLVKPGG
SLRLSCAASGVIFSDYYMAWVRQAPGKCLEWVASINFNADISYYRESVKG
RFTISRDDSKNTLYLQMNSLKTEDTAVYYCTTDANRQNYDWFAYWGQGTL
VTVSSGGGGSGGGGSGGGGSAIQLTQSPSSLSASVGDRVTITCKASESVS
SSMYSYMHWYQQKPGKAPKLLIYRASNLESGVPSRFSGSGSGTDFTLTIS
SLQPEDFATYYCQQSWTAPRTFGCGTKVEIKGGGGSGGGGSKNTVYLQMN
SLRAEDTAVYYCARTVPGYSTAPYFDLWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKKVEPKSC
CA645 Fab heavy chain with CA497 dsscFv (vLvH)
(bold and italics) inserted into framework 3,
linkers underlined:
(SEQ ID NO: 130)
EVQLLESGGGLVQPGGSLRLSCAVSGIDLSNYAINWVRQAPGKGLEWIGI
IWASGTTFYATWAKGRFTISRDNSGGGGSGGGGSAIQLTQSPSSLSASVG
DRVTITCKASESVSSSMYSYMHWYQQKPGKAPKLLIYRASNLESGVPSRF
SGSGSGTDFTLTISSLQPEDFATYYCQQSWTAPRTFGCGTKVEIKGGGGS
GGGGSGGGGSEVQLVESGGGLVKPGGSLRLSCAASGVIFSDYYMAWVRQA
PGKCLEWVASINFNADISYYRESVKGRFTISRDDSKNTLYLQMNSLKTED
TAVYYCTTDANRQNYDWFAYWGQGTLVTVSSGGGGSGGGGSKNTVYLQMN
SLRAEDTAVYYCARTVPGYSTAPYFDLWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKKVEPKSC

### Transient Cell Expression

Engineered Fab fragments were transiently expressed in either HEK-293 cells using ExpiFectamine 293 transfection kit (Life Technologies, according to the manufacturer's instructions) or CHO-S XE cells, a CHO-K1 derived cell line (Cain K et al, A CHO cell line engineered to express XBP1 and ERO1-Lα has increased levels of transient protein expression.

Biotechnology Progress 2013; 29: 697-706), using ExpiFectamine CHO transfection kit (Life Technologies, according to the manufacturer's instructions). HEK-293 cells were used for small scale expression (2 ml) to prepare antibody constructs for direct SPR analysis. CHO-S XE cells were used for large scale expression (200 ml) to prepare antibody constructs for purification prior to further characterization.

### Purification

Affinity chromatography was used to purify Fab fragments from culture supernatants. Supernatants containing the antibodies were passed over a HiTrap Protein G column (GE Healthcare). Following a washing step with phosphate buffered saline (PBS) pH 7.4, the bound material was eluted with a gradient of PBS pH 7.4 versus an increasing percentage by volume of 0.1M phosphate citrate buffer pH 2.6. The eluate was neutralized with 2 M Tris-HCl (pH 8). Fractions were pooled, quantified by absorbance at 280 nm, and concentrated using Amicon Ultra centrifugal filters (Merck Millipore). To isolate the monomeric fractions of the constructs, size exclusion chromatography over a HiLoad 16/60, Superdex 200 column (GE Healthcare) equilibrated with PBS pH 7.4, was used. Fractions containing monomeric Fab were pooled, quantified, concentrated and stored at 4° C.

### UPLC (Ultra-High-Performance Liquid Chromatography):

2 μg of sample was injected onto a SEC BEH200 column, pre-equilibrated in PBS, pH7.4 and run for 10 mins. Fluorescence intensity was recorded against elution time and peak areas picked manually in EMpower software.

The UPLC elution profiles were obtained and analysed. Peaks appeared >98% monomeric (99,42% for CA645 Fab-Fwk3 CA497 dsscFv (vLvH), 99,51% for CA645 Fab-Fwk3 CA497 dsscFv (vHvL)). The controls CA497 dsscFv and CA645 Fab showed respectively 99,99% and 98.43% of multimeric forms (data not shown). Accordingly, the purified proteins were homogenous and lacked multimeric forms or aggregates.

### Molecular Stability

The molecular stability was measured by melting temperature (Tm) (measure of unfolding) analysis by Differential Scanning Calorimetry (DSC) method. Thermograms were acquired on an automated MicroCal VP DSC (Malvern Panalytical) from 0-100° C., using a step rate of 1° C./min, with a pre-incubation scan time of 15 min, a filtering period of 5s and feedback set to passive. The data was buffer subtracted before a manual baseline subtraction and concentration correction. The thermograms were fitted to a non-2-state model in Origin7.0.

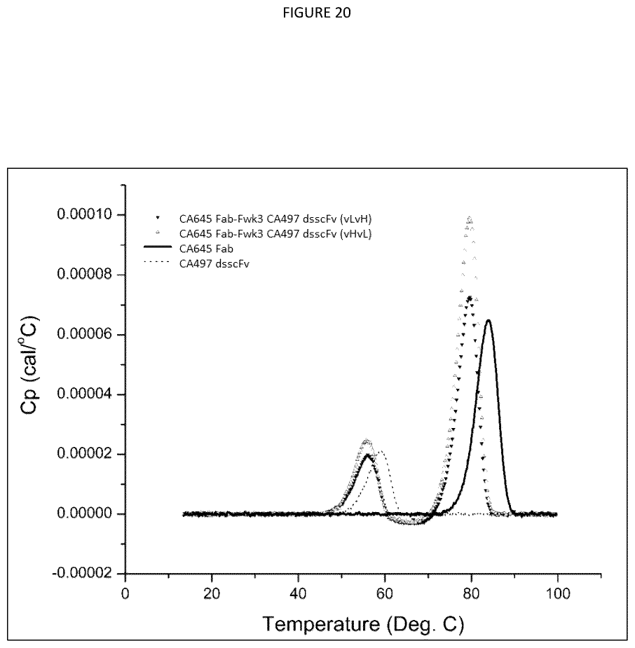

The results are shown in FIG. 20 (DSC curves; vertical axis: (Cp) specific heat; horizontal axis: temperature in Degrees C. (° C.)) and in the table 3 below. CA497 dsscFv and CA645 Fab showed single transitions of 58.40° C. and 83.57° C. respectively. When combined in the Fab-Fwk3 inserted dsscFv format, the CA497 dsscFv Tm is almost identical (difference of 2-3° C.) and the CA645 Fab has a variation of Tm of about 4-5° C. Accordingly, the pairing of CA497 dsscFv and CA645 Fab in this antibody format according to the invention does not impact significantly the conformational stability of either domain.

TABLE 3
Unfolding transition temperatures

| Sample | Tm1 (° C.) | Tm2 (° C.) |
|---|---|---|
| CA645 Fab | 83.57 | |
| CA497 dsscFv | 58.40 | |
| CA645 Fab heavy chain | 55.59 | 78.64 |
| with CA497 dsscFv (vLvH) | | |
| inserted into Fwk3 | | |
| CA645 Fab heavy chain | 55.41 | 78.73 |
| with CA497 dsscFv (vHvL) | | |
| inserted into Fwk3 | | |

### Neutralisation Assay

The potency of antibody CA497 IgG4, CA645 Fab-Fwk3 CA497 dsscFv (vHvL) and CA645 Fab-Fwk3 CA497 dsscFv (vLvH) against human recombinant IL-17A in Hela cells was tested, in presence or in absence of HSA. Hela cells were obtained from the cell bank at ATCC (ATCC CCL-2). Cells were grown in Dulbecco's modified Eagle's medium (DMEM) supplemented with 10% foetal calf serum, penicillin, gentamycin and glutamine. 1×10⁴ cells were plated out into 96 well flat bottomed tissue culture plates. Cells were incubated overnight and washed once in assay buffer. Human IL-17A (25 ng ml⁻¹) was incubated in the presence of a fixed concentration of human TNF-α, and this mixture was preincubated with the tested antibody. The cytokines and antibody were then added to the Hela cells to stimulate the cells which were incubated overnight. In the Hela cell line, IL-17A synergises with TNF-alpha to induce the production of IL-6 which can be quantified, e.g. by ELISA. The production of IL-6 in the cell culture supernatant is proportionate to the amount of IL-17A added to the cells. Human IL-6 levels were measured by ELISA and quantified by comparison with known standard concentrations of human IL-6. In this assay, the inhibition of the secretion of IL-6 is correlated with the inhibition of IL-17A.

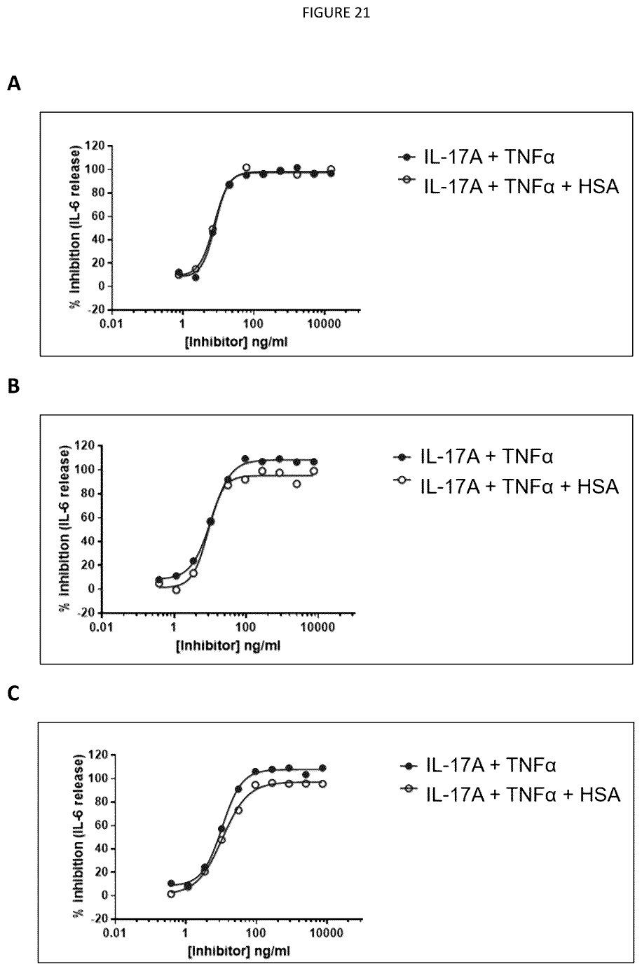

The inhibition curves are shown in FIG. 21 (horizontal axis: antibody (inhibitor) concentration in ng/ml, vertical axis: inhibition of the secretion of IL-6 (%)) and indicate that the parent antibody CA497 IgG4 and both antibody fusion constructs potently neutralised human recombinant IL-17A. In addition, the results indicate that the dsscFv anti-IL-17 retained functionality when inserted into the Fwk3 region of the Fab anti-HSA. The EC50 values calculated from these experiments (table 4 below) indicated that antibody CA645 Fab-Fwk3 CA497 dsscFv (vLvH) and CA645 Fab-Fwk3 CA497 dsscFv (vHvL) gave an EC₅₀ of 10.5 ng/ml and 10.6 ng/ml respectively against human recombinant IL-17A (25 ng.ml⁻¹) and that the neutralisation potency was not affected by the presence of HSA (EC₅₀ of 10.8 and 8.7 ng/ml respectively, in presence of HSA). Also, the CA645 Fab-Fwk3 CA497 dsscFv constructs had a neutralisation potency comparable to the one of the parent CA497 IgG4 antibody.

TABLE 4
EC50 values for the neutralisation of IL-17A in
presence or absence of HSA

| | HSA (2.5%) | IL-17A EC50 (ng/ml) |
|---|---|---|
| CA497 IgG4 | − | 8.1 |
| CA497 IgG4 | + | 7.6 |
| CA645 Fab-Fwk3 CA497 | − | 10.5 |
| dsscFv (vLvH) | | |
| CA645 Fab-Fwk3 CA497 | + | 10.8 |
| dsscFv (vLvH) | | |
| CA645 Fab-Fwk3 CA497 | − | 10.6 |
| dsscFv (vHvL) | | |
| CA645 Fab-Fwk3 CA497 | + | 8.7 |
| dsscFv (vHvL) | | |

### Example 14—Full CA645 IgG with a dsscFv Inserted into Framework 3

A fusion protein was generated and expressed in accordance with the above methods to insert CA497 dsscFv (vH-vL) or CA497 dsscFv (vL-vH) into the framework 3 region of the CA645 IgG4P construct, as shown below. The resulting antibody fusion proteins may be termed CA645 IgG4P Fwk3-CA497 dsscFv (vHvL) and CA645 IgG4P Fwk3-CA497 dsscFv (vLvH).

### Gene Design

Genes encoding light and heavy chain V-regions, including variants with a framework 3 insertion, were designed and constructed by an automated synthesis approach (ATUM). Light chain V-region genes (e.g. coding for the CA645 VL domain of SEQ ID NO: 2) were cloned into expression vectors containing DNA encoding human Cx region (Km3 allotype). Heavy chain V-region genes were cloned into expression vectors containing DNA encoding IgG4P constant regions for full-length antibody.

CA645 IgG4P heavy chain with CA497 dsscFv (vHvL)
(bold and italics) inserted into framework 3,
linkers underlined:
(SEQ ID NO: 131)
EVQLLESGGGLVQPGGSLRLSCAVSGIDLSNYAINWVRQAPGKGLEWIGI
IWASGTTFYATWAKGRFTISRDNSGGGGSGGGGSEVQLVESGGGLVKPGG
SLRLSCAASGVIFSDYYMAWVRQAPGKCLEWVASINFNADISYYRESVKG
RFTISRDDSKNTLYLQMNSLKTEDTAVYYCTTDANRQNYDWFAYWGQGTL
VTVSSGGGGSGGGGSGGGGSAIQLTQSPSSLSASVGDRVTITCKASESVS
SSMYSYMHWYQQKPGKAPKLLIYRASNLESGVPSRFSGSGSGTDFTLTIS
SLQPEDFATYYCQQSWTAPRTFGCGTKVEIKGGGGSGGGGSKNTVYLQMN
SLRAEDTAVYYCARTVPGYSTAPYFDLWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGK
CA645 IgG4P heavy chain with CA497 dsscFv (vLvH)
(bold and italics) inserted intoframework 3,
linkers underlined:
(SEQ ID NO: 132)
EVQLLESGGGLVQPGGSLRLSCAVSGIDLSNYAINWVRQAPGKGLEWIGI
IWASGTTFYATWAKGRFTISRDNSGGGGSGGGGSAIQLTQSPSSLSASVG
DRVTITCKASESVSSSMYSYMHWYQQKPGKAPKLLIYRASNLESGVPSRF
SGSGSGTDFTLTISSLQPEDFATYYCQQSWTAPRTFGCGTKVEIKGGGGS
GGGGSGGGGSEVQLVESGGGLVKPGGSLRLSCAASGVIFSDYYMAWVRQA
PGKCLEWVASINFNADISYYRESVKGRFTISRDDSKNTLYLQMNSLKTED
TAVYYCTTDANRQNYDWFAYWGQGTLVTVSSGGGGSGGGGSKNTVYLQMN
SLRAEDTAVYYCARTVPGYSTAPYFDLWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGK

### Transient Cell Expression

Engineered antibodies were transiently expressed in either HEK-293 cells using ExpiFectamine 293 transfection kit (Life Technologies, according to the manufacturer's instructions) or CHO-S XE cells, a CHO-K1 derived cell line, using ExpiFectamine CHO transfection kit (Life Technologies, according to the manufacturer's instructions). HEK-293 cells were used for small scale expression (2 ml) to prepare antibody constructs for direct SPR analysis. CHO-S XE cells were used for large scale expression (200 ml) to prepare antibody constructs for purification prior to further characterization.

### Purification

Affinity chromatography was used to purify IgG from culture supernatants. Supernatants containing IgG were passed over a MabSelect SuRe column (GE Healthcare). Following a washing step with phosphate buffered saline (PBS) pH 7.4, the bound material was eluted with a gradient of PBS pH 7.4 versus an increasing percentage by volume of 0.1M phosphate citrate buffer pH 2.6. The eluate was neutralized with 2 M Tris-HCl (pH 8). Fractions were pooled, quantified by absorbance at 280 nm, and concentrated using Amicon Ultra centrifugal filters (Merck Millipore). To isolate the monomeric fractions of IgG-based constructs, size exclusion chromatography over a HiLoad 16/60, Superdex 200 column (GE Healthcare) equilibrated with PBS pH 7.4, was used. Fractions containing monomeric IgG constructs were pooled, quantified, concentrated and stored at 4° C.

### Simultaneous Binding to IL-17 and HSA

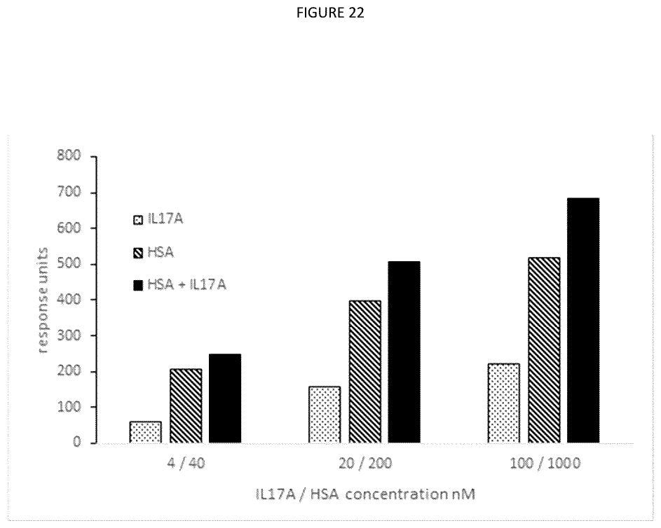

In order to test simultaneous binding of IL-17A and HSA to CA645 IgG4P Fwk3-CA497 dsscFv (vHvL), the latter was immobilised on a Biacore chip in place of polyclonal goat F(ab)₂ in the same procedure as previously. Three concentrations of both IL-17A and HSA were selected to give low, medium and high levels of binding to the immobilized antibody, specifically these corresponded to stoichiometric ratios of ligand to antibody in the ranges 0.15 to 0.3, 0.4 to 0.6 and 0.65 to 0.75 respectively. In the case of IL-17A concentrations were 4, 20 and 100 nM and for HSA concentrations were 40, 200 and 1000 nM respectively. The above low, medium and high concentrations, were tested by SPR as previously described in individual cycles either as separate ligands or as IL17A/HSA mixtures and report points corrected by subtraction of buffer control cycles. The results are shown in FIG. 22 and show that the CA645 IgG4P Fwk3-CA497 dsscFv (vHvL) bind both IL-17A and HSA, in a concentration dependent manner. FIG. 22 further shows that in the presence of both binding partners, the signal is additive, showing that binding to both IL-17A and HSA can occur simultaneously.

### Example 15—Antibodies of the Invention which Bind to Both CD3 and CD28

### Gene Design

The antibodies were generated and expressed in accordance with the methods described above to insert anti-CD3 dsscFv (vH-vL) into the framework 3 region of the anti-CD3 or anti-CD28 scaffold antibody (IgG4P or Fab fragment) as shown below in Table 5. The resulting antibody constructs according to the invention may be termed anti-CD28 Fab Fwk3-anti-CD3 dsscFv (LC of SEQ ID NO: 136, HC of SEQ ID NO: 139), anti-CD3 Fab Fwk3-anti-CD3 dsscFv (LC of SEQ ID NO: 133, HC of SEQ ID NO: 140), anti-CD28 IgG4P Fwk3-anti-CD3 dsscFv (LC of SEQ ID NO: 136, HC of SEQ ID NO: 141), anti-CD3 IgG4P Fwk3-anti-CD3 dsscFv (LC of SEQ ID NO: 133, HC of SEQ ID NO: 142).

TABLE 5
Antibody Constructs

| | Antigen | Antigen | |
|---|---|---|---|
| | specificity | specificity | |
| | (V-domain | (dsscFv | |
| Scaffold | of the | inserted | |
| antibody | scaffold) | into Fwk3) | Sequence |
| Fab | CD3 | none | Light chain of SEQ ID NO: 133 |
| | | | Heavy chain of SEQ ID NO: 134 |
| Fab | CD28 | none | Light chain of SEQ ID NO: 136 |
| | | | Heavy chain of SEQ ID NO: 137 |
| Fab | CD28 | CD3 | Light chain of SEQ ID NO: 136 |
| | | | Heavy chain of SEQ ID NO: 139 |
| Fab | CD3 | CD3 | Light chain of SEQ ID NO: 133 |
| | | | Heavy chain of SEQ ID NO: 140 |
| IgG4P | CD3 | none | Light chain of SEQ ID NO: 133 |
| | | | Heavy chain of SEQ ID NO: 135 |
| IgG4P | CD28 | none | Light chain of SEQ ID NO: 136 |
| | | | Heavy chain of SEQ ID NO: 138 |
| IgG4P | CD28 | CD3 | Light chain of SEQ ID NO: 136 |
| | | | Heavy chain of SEQ ID NO: 141 |
| IgG4P | CD3 | CD3 | Light chain of SEQ ID NO: 133 |
| | | | Heavy chain of SEQ ID NO: 142 |

Anti-CD3 Light chain, muromonab (humanised OKT3)
(Variable domain underlined; CDRs in bold and
italics):
(SEQ ID NO: 133)
DIQMTQSPSSLSASVGDRVTITCSASSSVSYMNWYQQTPGKAPKLLIYD
TSKLASGVPSRFSGSGSGTDYTFTISSLQPEDIATYYCQQWSSNPFTFG
QGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
Anti-CD3 Heavy chain Fab, CH1 from IgG1, muromonab
(humanised OKT3) (Variable domain underlined; CDRs
in bold and italics):
(SEQ ID NO: 134)
QVQLVQSGGGVVQPGRSLRLSCKASGYTFTRYTMHWVRQAPGKGLEWIG
YINPSRGYTNYNQKFKDRFTISRDNSKNTAFLQMDSLRPEDTGVYFCAR
YYDDHYCLDYWGQGTPVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSC
Anti-CD3 Heavy chain IgG4P, muromonab (humanised
OKT3) (Variable domain underlined; CDRs in bold
and italics):
(SEQ ID NO: 135)
QVQLVQSGGGVVQPGRSLRLSCKASGYTFTRYTMHWVRQAPGKGLEWIG
YINPSRGYTNYNQKFKDRFTISRDNSKNTAFLQMDSLRPEDTGVYFCAR
YYDDHYCLDYWGQGTPVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSLGK
Anti-CD28 Light chain, theralizumab (Variable
domain underlined; CDRs in bold and italics):
(SEQ ID NO: 136)
DIQMTQSPSSLSASVGDRVTITCHASQNIYVWLNWYQQKPGKAPKLLIY
KASNLHTGVPSRFSGSGSGTDFTLTISSLQPEDFATYYCQQGQTYPYTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Anti-CD28 Heavy chain Fab, CH1 from IgG1,
theralizumab (Variable domain underlined; CDRs
in bold and italics):
(SEQ ID NO: 137)
QVQLVQSGAEVKKPGASVKVSCKASGYTFTSYYIHWVRQAPGQGLEWIG
CIYPGNVNTNYNEKFKDRATLTVDTSISTAYMELSRLRSDDTAVYFCTR
SHYGLDWNFDVWGQGTTVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSC
Anti-CD28 Heavy chain IgG4P, theralizumab
(Variable domain underlined; CDRs in bold and
italics):
(SEQ ID NO: 138)
QVQLVQSGAEVKKPGASVKVSCKASGYTFTSYYIHWVRQAPGQGLEWIG
CIYPGNVNTNYNEKFKDRATLTVDTSISTAYMELSRLRSDDTAVYFCTR
SHYGLDWNFDVWGQGTTVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGK
Anti-CD28 Fab heavy chain with anti-CD3 dsscFv
(vHvL) (bold and italics) inserted into framework
3, linkers underlined:
(SEQ ID NO: 139)
QVQLVQSGAEVKKPGASVKVSCKASGYTFTSYYIHWVRQAPGQGLEWIG
CIYPGNVNTNYNEKFKDRATLTVDTSGGGGSGGGGSQVQLVQSGGGVVQ
PGRSLRLSCKASGYTFTRYTMHWVRQAPGKCLEWIGYINPSRGYTNYNQ
KFKDRFTISRDNSKNTAFLQMDSLRPEDTGVYFCARYYDDHYCLDYWGQ
GTPVTVSSGGGGSGGGGSGGGGSGGGGSDIQMTQSPSSLSASVGDRVTI
TCSASSSVSYMNWYQQTPGKAPKLLIYDTSKLASGVPSRFSGSGSGTDY
TFTISSLQPEDIATYYCQQWSSNPFTFGCGTKLEIKGGGGSGGGGSIST
AYMELSRLRSDDTAVYFCTRSHYGLDWNFDVWGQGTTVTVSSASTKGPS
VFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSC
Anti-CD3 Fab heavy chain with anti-CD3 dsscFv
(vHvL) (bold and italics) inserted into framework
3, linkers underlined:
(SEQ ID NO: 140)
QVQLVQSGGGVVQPGRSLRLSCKASGYTFTRYTMHWVRQAPGKGLEWIG
YINPSRGYTNYNQKFKDRFTISRDNSGGGGSGGGGSQVQLVQSGGGVVQ
PGRSLRLSCKASGYTFTRYTMHWVRQAPGKCLEWIGYINPSRGYTNYNQ
KFKDRFTISRDNSKNTAFLQMDSLRPEDTGVYFCARYYDDHYCLDYWGQ
GTPVTVSSGGGGSGGGGSGGGGSGGGGSDIQMTQSPSSLSASVGDRVTI
TCSASSSVSYMNWYQQTPGKAPKLLIYDTSKLASGVPSRESGSGSGTDY
TFTISSLQPEDIATYYCQQWSSNPFTFGCGTKLEIKGGGGSGGGGSKNT
AFLQMDSLRPEDTGVYFCARYYDDHYCLDYWGQGTPVTVSSASTKGPSV
FPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSC
Anti-CD28 IgG4P heavy chain with anti-CD3 dsscFv
(vHvL) (bold and italics) inserted into framework
3, linkers underlined:
(SEQ ID NO: 141)
QVQLVQSGAEVKKPGASVKVSCKASGYTFTSYYIHWVRQAPGQGLEWIG
CIYPGNVNTNYNEKFKDRATLTVDTSGGGGSGGGGSQVQLVQSGGGVVQ
PGRSLRLSCKASGYTFTRYTMHWVRQAPGKCLEWIGYINPSRGYTNYNQ
KFKDRFTISRDNSKNTAFLQMDSLRPEDTGVYFCARYYDDHYCLDYWGQ
GTPVTVSSGGGGSGGGGSGGGGSGGGGSDIQMTQSPSSLSASVGDRVTI
TCSASSSVSYMNWYQQTPGKAPKLLIYDTSKLASGVPSRFSGSGSGTDY
TFTISSLQPEDIATYYCQQWSSNPFTFGCGTKLEIKGGGGGGGGSISTA
YMELSRLRSDDTAVYFCTRSHYGLDWNFDVWGQGTTVTVSSASTKGPSV
FPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPC
PPCPAPEFLGGPSVFLFPPKPKDTLMISRTPEVTCVVVDVSQEDPEVQF
NWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNV
FSCSVMHEALHNHYTQKSLSLSLGK
Anti-CD3 IgG4P heavy chain with anti-CD3 dsscFv
(vHvL) (bold and italics) inserted into framework
3, linkers underlined:
(SEQ ID NO: 142)
QVQLVQSGGGVVQPGRSLRLSCKASGYTFTRYTMHWVRQAPGKGLEWIG
YINPSRGYTNYNQKFKDRFTISRDNSGGGGSGGGGSQVQLVQSGGGVVQ
PGRSLRLSCKASGYTFTRYTMHWVRQAPGKCLEWIGYINPSRGYTNYNQ
KFKDRFTISRDNSKNTAFLQMDSLRPEDTGVYFCARYYDDHYCLDYWGQ
GTPVTVSSGGGGSGGGGSGGGGSGGGGSDIQMTQSPSSLSASVGDRVTI
TCSASSSVSYMNWYQQTPGKAPKLLIYDTSKLASGVPSRFSGSGSGTDY
TFTISSLQPEDIATYYCQQWSSNPFTFGCGTKLEIKGGGGSGGGGSKNT
AFLQMDSLRPEDTGVYFCARYYDDHYCLDYWGQGTPVTVSSASTKGPSV
FPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPC
PPCPAPEFLGGPSVFLFPPKPKDTLMISRTPEVTCVVVDVSQEDPEVQF
NWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNV
FSCSVMHEALHNHYTQKSLSLSLGK

### UPLC (Ultra-High-Performance Liquid Chromatography):

2 μg of sample was injected onto a SEC BEH200 column, pre-equilibrated in PBS, pH7.4 and run for 10 mins. Fluorescence intensity was recorded against elution time and peak areas picked manually in EMpower software.

The UPLC elution profiles were obtained and analysed. Peaks appeared >98% monomeric (anti-CD3 IgG4P Fwk3-anti-CD3 dsscFv: 99,02%, anti-CD3 Fab Fwk3-anti-CD3 dsscFv: 99,79%, anti-CD28 Fab Fwk3-anti-CD3 dsscFv: 99,48%) with the exception of a lower molecular weight shoulder of 12.12% in anti-CD28 IgG4P Fwk3-anti-CD3 dsscFv. There was no evidence of multimerization of any format.

### Molecular Stability

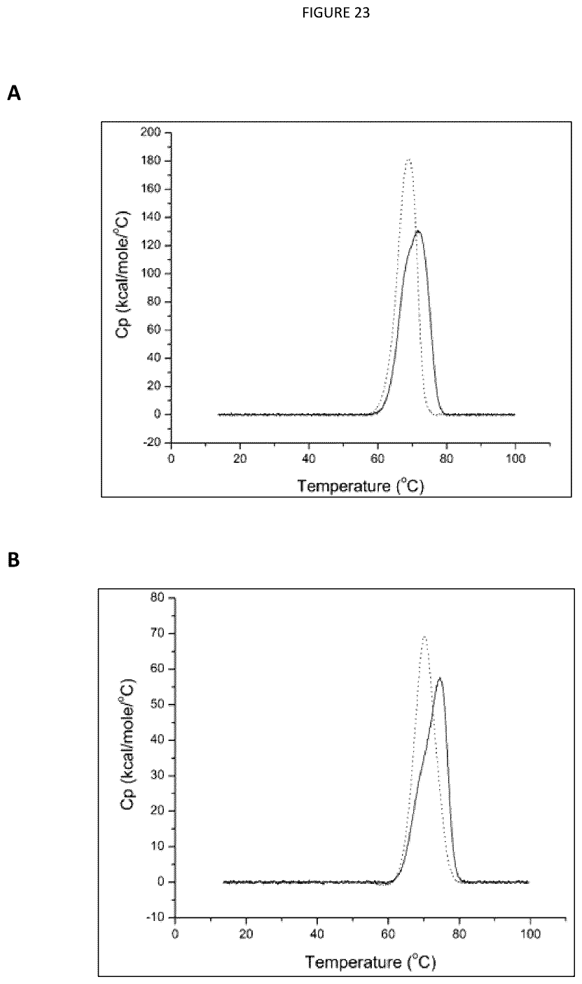

The molecular stability was measured by melting temperature (Tm) (measure of unfolding) analysis by Differential Scanning Calorimetry (DSC) method as described above. The DSC curves are shown in FIG. 23 (A) and (B), and the Tm values presented in Table 6 and 7 below.

TABLE 6
Fab-Fwk3-dsscFv

| Antibody of the invention | Tm1 (° C.) | Tm2 (° C.) |
|---|---|---|
| Anti-CD28 Fab Fwk3-anti | 70.16 | 74.73 |
| CD3 dsscFv | | |
| Anti-CD3 Fab Fwk3-anti- | 70.04 | |
| CD3 dsscFv | | |

The DSC curves for anti-CD3 Fab Fwk3-anti-CD3 dsscFv showed a single unfolding transition at 70.04° C. In contrast, the unfolding of the construct containing anti-CD28 cannot be accurately described by a single transition but fit better to a non-2-state, two transition model; the lowest of which can be attributed to the dsscFv component. The results notably show that inserting the Fab in the framework 3 position resulted in stable constructs, as the values 65° C. −75° C. are in line with that of a typical IgG.

TABLE 7
IgG4P-Fwk3-dsscFv

| Antibody of the invention | Tm1 (° C.) | Tm2 (° C.) |
|---|---|---|
| Anti-CD28 IgG4P Fwk3-anti- | 68.97 | 73.28 |
| CD3 dsscFv | | |
| Anti-CD3 IgG4P Fwk3-anti- | 68.55 | |
| CD3 dsscFv | | |

The DSC curves for anti-CD3 IgG4P Fwk3-anti-CD3 dsscFv showed a single unfolding transition at 68.55° C. In contrast, the unfolding of constructs containing anti-CD28 cannot be accurately described by a single transition but fit better to a non-2-state, two transition model; the lowest of which can be attributed to the dsscFv component. The results notably show that inserting the IgG in the framework 3 position resulted in stable constructs, as the Tm values of 65° C.-75° C. are typical of IgG-like molecules.

Evaluation of Fab and IgG4P Constructs with Framework 3 Inserts in a Human Functional PBMC Assay

Constructs were tested for their ability to stimulate T cell activation in a culture of peripheral blood mononuclear cells (PBMC) according to the method described below.

### Reagents

Antibodies used to detect CD4 and CD8 T cells, distinguish live and dead cells and measure the activation marker CD69 were obtained from BD Biosciences (Anti-CD4 antibody, anti-CD8, anti-CD69 antibody and the antibody distinguishing Live/dead cells were coupled respectively to the fluorophores FITC, PE, BV421 and Near Infrared. All cell culture was performed in RPMI1640 (Thermo) plus 5% heat-inactivated human AB serum (Sigma) and 2 mM glutamine (Thermo). All flow cytometry staining was performed using a standard flow cytometry buffer comprising Dulbecco's PBS (Thermo) plus 1% bovine serum albumin (Sigma), 2 mM EDTA (Sigma) and 0.1% sodium azide (Sigma). Human OKT3 (Ebioscience) and T cell Transact (Miltenyi) a colloidal suspension of anti-CD3 and CD28. All flow cytometry data was acquired using an iQUEplus (Sartorius). T cells were identified using Forecyt software (Sartorius) and data metrics imported into GraphPad Prism 7 (GraphPad) for data transformations and graphical representation.

### Method

Antibody constructs in table 5 were diluted down from 25 μg/ml in eight 1 in 5 dilution steps. In the post analysis step all these values were converted into nM concentrations to allow equal comparisons of all constructs. Into 96 well ‘U’ bottomed tissue culture plates 1.5×10⁵ PBMC (from two different donors anonymised as donor 1 and 2) were mixed with the diluted antibodies. OKT3 (5 μg/ml), Transact (CD3/CD28 diluted 1 in 100) and media alone were reference controls for the experiment. After mixing, plates were incubated at 37 degrees Celsius for 24 hours. After this time, plates were cooled on ice and spun (all subsequent spins were at 500g for 5 mins at 4 degrees Celsius) and supernatant removed from each well. Plates were washed again, supernatant aspirated and 20 μl of an antibody cocktail added to each well (all antibodies were diluted 1:20 in flow cytometry buffer and 1 μl of live/dead stain added per lml of cocktail). Plates were left on ice in the dark for 1 hour. After this time plates were washed a further two times and supernatant removed from all wells. Plates were finally resuspended in 20 μl of flow cytometry buffer and at least 10,000 cells per well acquired.

### Results

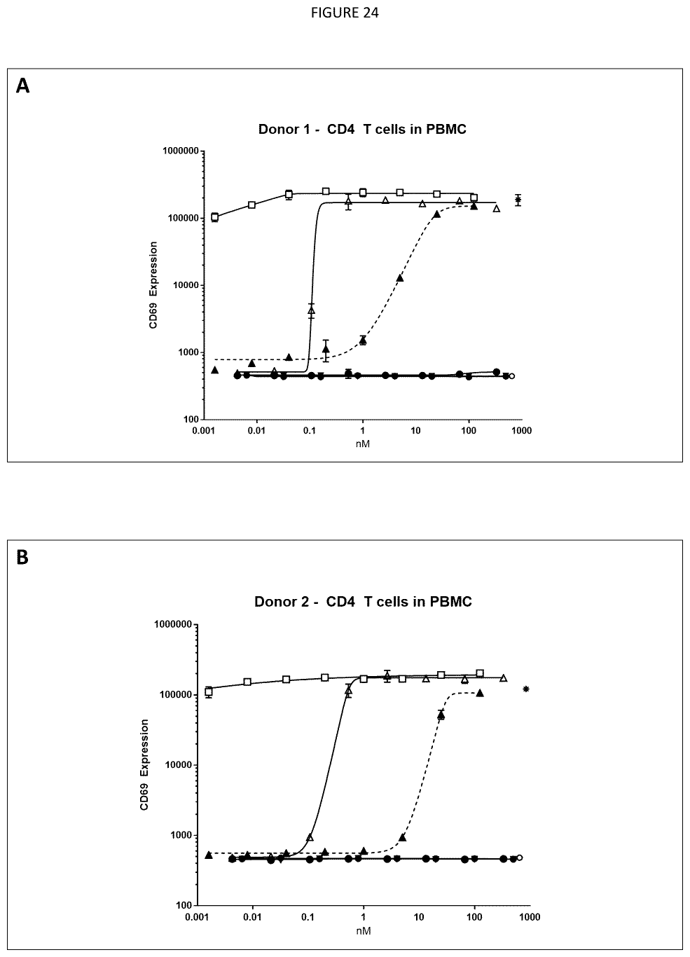

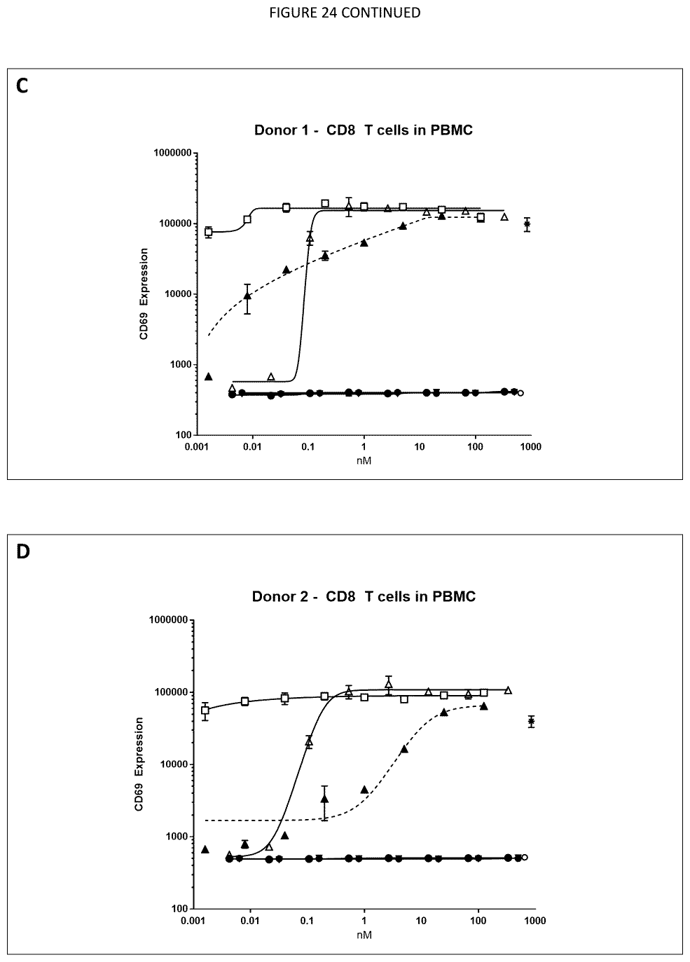

The results are shown in FIG. 24 (A-D; the vertical axis represents the level of expression of CD69 (MFI) and the horizontal axis represents the concentration of antibody construct in nM). The experiment was deemed successful since the positive control (OKT3) induced the early activation marker, CD69, on both CD4 and CD8 T cells. The minimum (cells only) level of activation was determined by the cells only control. As can be seen from FIG. 24(A) and (B), the induction of CD69 on CD4 T cells for the tested constructs was consistent between donors.

The antibodies of the invention containing CD3 and CD28 (either in the Fab or IgG4P format) were the most potent activators of CD4 T cells. As expected in this assay constructs containing an Fc can also bring in added activity via Fc receptors on other (non) T cells, in particular monocytes, which give enhanced costimulatory signals. In this respect the anti-CD3 IgG4P Fwk3-anti-CD3dsscFv also showed activity in this assay (as does the OKT3 control which is a whole IgG anti-CD3 antibody). This data demonstrates that antibodies of the invention which bind to both CD3 and CD28 are functional activators of human CD4 T cells in a mixed PBMC assay, and as such are capable of exercising the function of a bispecific antibody.

In addition, activation of CD8 T cells was also measured in the same assay. As described previously all positive and negative controls performed as expected. As can be seen from FIG. 24 (C) and (D), the induction of CD69 on CD8 T cells for the tested constructs was consistent between donors. The antibodies of the invention containing CD3 and CD28 (either in the Fab or IgG4P format) were the most potent activators of CD8 T cells. The anti-CD3 IgG4P Fwk3 anti-CD3dsscFv also showed activity in this assay (as does the OKT3 control which is a whole IgG anti-CD3 antibody). This data demonstrates that antibodies of the invention which bind to both CD3 and CD28 are functional activators of human CD8 T cells in a mixed PBMC assay, and as such are capable of exercising the function of a bispecific antibody.

The results show that antibodies according to the invention have the functionality of both the scaffold antibody (e.g. anti-CD28 Fab or IgG4P), and the insert polypeptide, i-e the second antibody (e.g. anti-CD3 dsscFv) when the insert polypeptide is present in the Fwk3 region of the scaffold antibody.

Comprising in the context of the present specification is intended to mean including.

Where technically appropriate, embodiments of the invention may be combined. Embodiments are described herein as comprising certain features/elements. The disclosure also extends to separate embodiments consisting or consisting essentially of said features/elements.

It will of course be understood that, although the present invention has been described by way of example, the examples are in no way meant to be limiting, and modifications can be made within the scope of the claims hereinafter. Preferred features of each embodiment of the invention are as for each of the other embodiments mutatis mutandis. All publications, including but not limited to patents and patent applications, cited in this specification are herein incorporated by reference as if each individual publication was specifically and individually indicated to be incorporated by reference herein.

1. An antibody comprising a variable (V) domain and an insert polypeptide, wherein the insert polypeptide is within the framework 3 region of the V domain, wherein the insert polypeptide is a cytokine or a second antibody.

2. The antibody according to claim 1, wherein:
(a) the V domain is a VH domain; or
(b) the V domain is a VH domain, which further comprises a VL domain; or
(c) the V domain is a VL domain; or
(d) the V domain is a VL domain, which further comprises a VH domain.

3-14. (canceled)

15. The antibody according to claim 1, which is:
(a) a full-length antibody;
(b) a full-length IgG: or
(c) a Fab, Fab′, F(ab′)₂, VHH, or scFv.

16-18. (canceled)

19. The antibody according to claim 1, which is humanised.

20. The antibody according to claim 1, wherein said V domain binds to human serum albumin.

21. The antibody according to claim 20, which comprises a VL domain comprising CDR L1 having the sequence set forth in SEQ ID NO: 3; CDR-L2 having the sequence set forth in SEQ ID NO: 4; and CDR-L3 having the sequence set forth in SEQ ID NO: 5.

22. The antibody according to claim 21, which comprises;
(a) a VL domain comprising the sequence set forth in SEQ ID NO: 2 or SEQ ID NO: 102; or
(b) a L chain comprising the sequence set forth in SEQ ID NO: 1.

23. (canceled)

24. The antibody according to claim 20, which comprises;
(a) a VH domain comprising CDR-H1 having the sequence set forth in SEQ ID NO: 8; CDR-H2 having the sequence set forth in SEQ ID NO: 9; and CDR-H3 having the sequence set forth in SEQ ID NO: 10;
(b) a VH domain comprising the sequence set forth in SEQ ID NO: 7 or SEQ ID NO: 103; or
(c) a H chain comprising the sequence set forth in SEQ ID NO: 6.

25-27. (canceled)

28. The antibody according to claim 1, wherein the cytokine is selected from IL-10, IL-15, IL-2, G-CSF, GM-CSF and EPO.

29. (canceled)

30. The antibody according to claim 1, wherein the second antibody is an scFv or a domain antibody.

31. The antibody according to claim 30, wherein the domain antibody is a VHH comprising a sequence selected from SEQ ID NOs: 67, 69 and 89.

32. The antibody according to claim 30, wherein:
(a) the second antibody binds to the same antigen as the V domain; or
(b) the second antibody binds to the same antigen as the V domain and the second antibody binds to a different epitope compared to the V domain.

33. (canceled)

34. The antibody according to claim 30, wherein the second antibody binds to a different antigen to the antibody comprising a V domain.

35. The antibody according to claim 30, wherein the second antibody binds to IL-17 and the antibody comprising a V domain binds to HSA; or wherein the second antibody binds to CD3 and the antibody comprising a V domain binds to CD28; or wherein the second antibody binds to CD28 and the antibody comprising a V domain binds to CD3.

36-39. (canceled)

40. An isolated DNA molecule or a pair of DNA molecules encoding the antibody according to claim 1.

41. A cloning or expression vector or a pair of cloning or expression vectors encoding the antibody according to claim 1.

42. A host cell comprising a cloning or expression vector or a pair of cloning of expression vectors according to claim 41.

43. A process for the production of an antibody as defined in claim 1, comprising culturing the host cell of claim 42 and isolating the antibody.

44. A pharmaceutical composition comprising an antibody as defined in claim 1 in combination with one or more of a pharmaceutically acceptable excipient, diluent or carrier.

45. (canceled)

46. A method of therapy comprising administering a therapeutically effective amount of the antibody as defined in claim 1.

47. (canceled)


SEQUENCE LISTING (144 sequences)

SEQ ID NO: 1 - protein, 217 aa, synthetic construct
  REGION 1..217: note = CA645 Light Chain gL5
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 110 aa, synthetic construct
  REGION 1..110: note = CA645 VL domain (gL5)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIK

SEQ ID NO: 3 - protein, 12 aa, synthetic construct
  REGION 1..12: note = CA645 CDR L1
QSSPSVWSNFLS

SEQ ID NO: 4 - protein, 7 aa, synthetic construct
  REGION 1..7: note = CA645 CDR L2
EASKLTS

SEQ ID NO: 5 - protein, 11 aa, synthetic construct
  REGION 1..11: note = CA645 CDR L3
GGGYSSISDTT

SEQ ID NO: 6 - protein, 224 aa, synthetic construct
  REGION 1..224: note = CA645 Heavy Chain gH5
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 7 - protein, 121 aa, synthetic construct
  REGION 1..121: note = CA645 VH domain (gH5)
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 8 - protein, 10 aa, synthetic construct
  REGION 1..10: note = CA645 CDR H1
GIDLSNYAIN

SEQ ID NO: 9 - protein, 16 aa, synthetic construct
  REGION 1..16: note = CA645 CDR H2
IIWASGTTFYATWAKG

SEQ ID NO: 10 - protein, 13 aa, synthetic construct
  REGION 1..13: note = CA645 CDR H3
TVPGYSTAPYFDL

SEQ ID NO: 11 - protein, 7 aa, synthetic construct
  REGION 1..7: note = Flexible linker
SGGGGSE

SEQ ID NO: 12 - protein, 6 aa, synthetic construct
  REGION 1..6: note = Flexible linker
DKTHTS

SEQ ID NO: 13 - protein, 6 aa, synthetic construct
  REGION 1..6: note = Flexible linker
SGGGGS

SEQ ID NO: 14 - protein, 11 aa, synthetic construct
  REGION 1..11: note = Flexible linker
SGGGGSGGGGS

SEQ ID NO: 15 - protein, 16 aa, synthetic construct
  REGION 1..16: note = Flexible linker
SGGGGSGGGGSGGGGS

SEQ ID NO: 16 - protein, 21 aa, synthetic construct
  REGION 1..21: note = Flexible linker
SGGGGSGGGGSGGGGSGGGGS

SEQ ID NO: 17 - protein, 26 aa, synthetic construct
  REGION 1..26: note = Flexible linker
SGGGGSGGGGSGGGGSGGGGSGGGGS

SEQ ID NO: 18 - protein, 11 aa, synthetic construct
  REGION 1..11: note = Flexible linker
AAAGSGGASAS

SEQ ID NO: 19 - protein, 16 aa, synthetic construct
  REGION 1..16: note = Flexible linker
  VARIANT 7: note = X is any naturally occurring amino acid
AAAGSGXGGGSGASAS

SEQ ID NO: 20 - protein, 21 aa, synthetic construct
  REGION 1..21: note = Flexible linker
  VARIANT 7: note = X is any naturally occurring amino acid
  VARIANT 12: note = X is any naturally occurring amino acid
AAAGSGXGGGSXGGGSGASAS

SEQ ID NO: 21 - protein, 26 aa, synthetic construct
  REGION 1..26: note = Flexible linker
  VARIANT 7: note = X is any naturally occurring amino acid
  VARIANT 12: note = X is any naturally occurring amino acid
  VARIANT 17: note = X is any naturally occurring amino acid
AAAGSGXGGGSXGGGSXGGGSGASAS

SEQ ID NO: 22 - protein, 31 aa, synthetic construct
  REGION 1..31: note = Flexible linker
  VARIANT 7: note = X is any naturally occurring amino acid
  VARIANT 12: note = X is any naturally occurring amino acid
  VARIANT 17: note = X is any naturally occurring amino acid
  VARIANT 22: note = X is any naturally occurring amino acid
AAAGSGXGGGSXGGGSXGGGSXGGGSGASAS

SEQ ID NO: 23 - protein, 13 aa, synthetic construct
  REGION 1..13: note = Flexible linker
  VARIANT 7: note = X is any naturally occurring amino acid
AAAGSGXSGASAS

SEQ ID NO: 24 - protein, 28 aa, synthetic construct
  REGION 1..28: note = Flexible linker
PGGNRGTTTTRRPATTTGSSPGPTQSHY

SEQ ID NO: 25 - protein, 11 aa, synthetic construct
  REGION 1..11: note = Flexible linker
ATTTGSSPGPT

SEQ ID NO: 26 - protein, 6 aa, synthetic construct
  REGION 1..6: note = Flexible linker
ATTTGS

SEQ ID NO: 27 - protein, 21 aa, synthetic construct
  REGION 1..21: note = Flexible linker
EPSGPISTINSPPSKESHKSP

SEQ ID NO: 28 - protein, 15 aa, synthetic construct
  REGION 1..15: note = Flexible linker
GTVAAPSVFIFPPSD

SEQ ID NO: 29 - protein, 15 aa, synthetic construct
  REGION 1..15: note = Flexible linker
GGGGIAPSMVGGGGS

SEQ ID NO: 30 - protein, 15 aa, synthetic construct
  REGION 1..15: note = Flexible linker
GGGGKVEGAGGGGGS

SEQ ID NO: 31 - protein, 15 aa, synthetic construct
  REGION 1..15: note = Flexible linker
GGGGSMKSHDGGGGS

SEQ ID NO: 32 - protein, 15 aa, synthetic construct
  REGION 1..15: note = Flexible linker
GGGGNLITIVGGGGS

SEQ ID NO: 33 - protein, 15 aa, synthetic construct
  REGION 1..15: note = Flexible linker
GGGGVVPSLPGGGGS

SEQ ID NO: 34 - protein, 12 aa, synthetic construct
  REGION 1..12: note = Flexible linker
GGEKSIPGGGGS

SEQ ID NO: 35 - protein, 18 aa, synthetic construct
  REGION 1..18: note = Flexible linker
RPLSYRPPFPFGFPSVRP

SEQ ID NO: 36 - protein, 18 aa, synthetic construct
  REGION 1..18: note = Flexible linker
YPRSIYIRRRHPSPSLTT

SEQ ID NO: 37 - protein, 18 aa, synthetic construct
  REGION 1..18: note = Flexible linker
TPSHLSHILPSFGLPTEN

SEQ ID NO: 38 - protein, 18 aa, synthetic construct
  REGION 1..18: note = Flexible linker
RPVSPFTFPRLSNSWLPA

SEQ ID NO: 39 - protein, 18 aa, synthetic construct
  REGION 1..18: note = Flexible linker
SPAAHFPRSIPRPGPIRT

SEQ ID NO: 40 - protein, 18 aa, synthetic construct
  REGION 1..18: note = Flexible linker
APGPSAPSHRSLPSRAFG

SEQ ID NO: 41 - protein, 18 aa, synthetic construct
  REGION 1..18: note = Flexible linker
PRNSIHFLHPLLVAPLGA

SEQ ID NO: 42 - protein, 18 aa, synthetic construct
  REGION 1..18: note = Flexible linker
MPSLSGVLQVRYLSPPDL

SEQ ID NO: 43 - protein, 18 aa, synthetic construct
  REGION 1..18: note = Flexible linker
SPQYPSPLTLTLPPHPSL

SEQ ID NO: 44 - protein, 18 aa, synthetic construct
  REGION 1..18: note = Flexiible linker
NPSLNPPSYLHRAPSRIS

SEQ ID NO: 45 - protein, 17 aa, synthetic construct
  REGION 1..17: note = Flexible linker
LPWRTSLLPSLPLRRRP

SEQ ID NO: 46 - protein, 18 aa, synthetic construct
  REGION 1..18: note = Flexible linker
PPLFAKGPVGLLSRSFPP

SEQ ID NO: 47 - protein, 18 aa, synthetic construct
  REGION 1..18: note = Flexible linker
VPPAPVVSLRSAHARPPY

SEQ ID NO: 48 - protein, 17 aa, synthetic construct
  REGION 1..17: note = Flexible linker
LRPTPPRVRSYTCCPTP

SEQ ID NO: 49 - protein, 18 aa, synthetic construct
  REGION 1..18: note = Flexible linker
PNVAHVLPLLTVPWDNLR

SEQ ID NO: 50 - protein, 18 aa, synthetic construct
  REGION 1..18: note = Flexible linker
CNPLLPLCARSPAVRTFP

SEQ ID NO: 51 - protein, 11 aa, synthetic construct
  REGION 1..11: note = Rigid linker
GAPAPAAPAPA

SEQ ID NO: 52 - protein, 4 aa, synthetic construct
  REGION 1..4: note = Rigid linker
PPPP

SEQ ID NO: 53 - protein, 112 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIN

SEQ ID NO: 54 - protein, 352 aa, synthetic construct
  REGION 1..352: note = CA645 Fab HC Fwk3 IL-15
EVQLLESGGGLVQPGGSLRLSCAVSGIDLSNYAINWVRQAPGKGLEWIGIIWASGTTFYATWAKGRFTISRDNKYQSGGSGMGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINSGGSYTYKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 133 aa, Homo sapiens [sequence omitted]

SEQ ID NO: 56 - protein, 368 aa, synthetic construct
  REGION 1..368: note = CA645 Fab HC Fwk3 IL-2
EVQLLESGGGLVQPGGSLRLSCAVSGIDLSNYAINWVRQAPGKGLEWIGIIWASGTTFYATWAKGRFTISRDNSGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 57 - protein, 178 aa, Homo sapiens
ATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 58 - protein, 412 aa, synthetic construct
  REGION 1..412: note = CA645 Fab HC Fwk3 G-CSF
EVQLLESGGGLVQPGGSLRLSCAVSGIDLSNYAINWVRQAPGKGLEWIGIIWASGTTFYATWAKGRFTISRDNSGGGGSATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPSGGGGKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 59 - protein, 127 aa, Homo sapiens [sequence omitted]

SEQ ID NO: 60 - protein, 362 aa, synthetic construct
  REGION 1..362: note = CA645 Fab HC Fwk3 GM-CSF
EVQLLESGGGLVQPGGSLRLSCAVSGIDLSNYAINWVRQAPGKGLEWIGIIWASGTTFYATWAKGRFTISRDNSGGGSGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 61 - protein, 359 aa, synthetic construct
  REGION 1..359: note = CA645 Fab HC with GM-CSF at C-terminus of CH1
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 62 - protein, 124 aa, Mus sp.
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 63 - protein, 359 aa, synthetic construct
  REGION 1..359: note = CA645 Fab HC Fwk3 mGM-CSF
EVQLLESGGGLVQPGGSLRLSCAVSGIDLSNYAINWVRQAPGKGLEWIGIIWASGTTFYATWAKGRFTISRDNSGGGSGGGGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - protein, 356 aa, synthetic construct
  REGION 1..356: note = CA645 Fab HC with mGM-CSF at C-terminus of CH1
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 65 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 66 - protein, 401 aa, synthetic construct
  REGION 1..401: note = CA645 Fab HC Fwk3 EPO
EVQLLESGGGLVQPGGSLRLSCAVSGIDLSNYAINWVRQAPGKGLEWIGIIWASGTTFYATWAKGRFTISRDNSGGGSGGGGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 67 - protein, 125 aa, synthetic construct
  REGION 1..125: note = VHH6
DVQFVESGGGSVHAGGSLRLNCATSGYIYSTYCMGWFRQAPGKEREGVAHIYTNSGRTYYADSVKGRFTISQDNAKNTVYLQMNSLKPEDTAIYYCAARPSIRCASFSATEYKDWGQGTQVTVSS

SEQ ID NO: 68 - protein, 369 aa, synthetic construct
  REGION 1..369: note = CA645 Fab HC Fwk3 VHH6
EVQLLESGGGLVQPGGSLRLSCAVSGIDLSNYAINWVRQAPGKGLEWIGIIWASGTTFYATWAKGRFTISRDNSGGGGSGGGGSDVQFVESGGGSVHAGGSLRLNCATSGYIYSTYCMGWFRQAPGKEREGVAHIYTNSGRTYYADSVKGRFTISQDNAKNTVYLQMNSLKPEDTAIYYCAARPSIRCASFSATEYKDWGQGTQVTVSSGGGGSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 69 - protein, 119 aa, synthetic construct
  REGION 1..119: note = VHH15
QVQLVESGGGSVQAGGSLRLSCVAASGYTGCTYDMRWYRQAPGKEREFVSGIDSDGRATYADSVKGRFTISQSNAKIAVYLQMDSLKLEDTAMYYCNLQCLRYPGEYYWGQGTQVTVSS

SEQ ID NO: 70 - protein, 363 aa, synthetic construct
  REGION 1..363: note = CA645 Fab HC Fwk3 VHH15
EVQLLESGGGLVQPGGSLRLSCAVSGIDLSNYAINWVRQAPGKGLEWIGIIWASGTTFYATWAKGRFTISRDNSGGGGSGGGGSQVQLVESGGGSVQAGGSLRLSCVAASGYTGCTYDMRWYRQAPGKEREFVSGIDSDGRATYADSVKGRFTISQSNAKIAVYLQMDSLKLEDTAMYYCNLQCLRYPGEYYWGQGTQVTVSSGGGGSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 71 - protein, 190 aa, Homo sapiens
QGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 72 - protein, 88 aa, synthetic construct
  REGION 1..88: note = Core domain of human sclerostin
CRELHFTRYVTDGPCRSAKPVTELVCSGQCGPARLLPNAIGRGKWWRPSGPDFRCIPDRYRAQRVQLLCPGGEAPRARKVRLVASCKC

SEQ ID NO: 73 - protein, 314 aa, synthetic construct
  REGION 1..314: note = CA645 Fab heavy chain with human sclerostin core domain inserted into framework 3, single S linkers
EVQLLESGGGLVQPGGSLRLSCAVSGIDLSNYAINWVRQAPGKGLEWIGIIWASGTTFYATWAKGRFTISRDNSSCRELHFTRYVTDGPCRSAKPVTELVCSGQCGPARLLPNAIGRGKWWRPSGPDFRCIPDRYRAQRVQLLCPGGEAPRARKVRLVASCKCSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 74 - protein, 333 aa, synthetic construct
  REGION 1..333: note = CA645 Fab heavy chain with human sclerostin core domain inserted into framework 3, Gly-Ser (2X G4S) linkers
EVQLLESGGGLVQPGGSLRLSCAVSGIDLSNYAINWVRQAPGKGLEWIGIIWASGTTFYATWAKGRFTISRDNSGGGGSGGGGSCRELHFTRYVTDGPCRSAKPVTELVCSGQCGPARLLPNAIGRGKWWRPSGPDFRCIPDRYRAQRVQLLCPGGEAPRARKVRLVASCKCSGGGGSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 75 - protein, 323 aa, synthetic construct
  REGION 1..323: note = CA645 Fab heavy chain with human sclerostin core domain inserted into framework 3, rigid linkers
EVQLLESGGGLVQPGGSLRLSCAVSGIDLSNYAINWVRQAPGKGLEWIGIIWASGTTFYATWAKGRFTISRDNSIPFTVCRELHFTRYVTDGPCRSAKPVTELVCSGQCGPARLLPNAIGRGKWWRPSGPDFRCIPDRYRAQRVQLLCPGGEAPRARKVRLVASCKCEYHGLQKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 76 - protein, 425 aa, synthetic construct
  REGION 1..425: note = CA645 Fab heavy chain with human mature full-length sclerostin inserted into framework 3, Gly-Ser (1X G4S) linkers
EVQLLESGGGLVQPGGSLRLSCAVSGIDLSNYAINWVRQAPGKGLEWIGIIWASGTTFYATWAKGRFTISRDNSGGGGSQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAYSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 77 - protein, 10 aa, synthetic construct
  REGION 1..10: note = Flexible linker
KYQSGGSGMG

SEQ ID NO: 78 - protein, 7 aa, synthetic construct
  REGION 1..7: note = Flexible linker
SGGSYTY

SEQ ID NO: 79 - protein, 8 aa, synthetic construct
  REGION 1..8: note = Flexible linker
GGGSGGGG

SEQ ID NO: 80 - protein, 7 aa, synthetic construct
  REGION 1..7: note = Flexible linker
GSSSSGS

SEQ ID NO: 81 - protein, 5 aa, synthetic construct
  REGION 1..5: note = Flexible linker
SGGGG

SEQ ID NO: 82 - protein, 5 aa, synthetic construct
  REGION 1..5: note = Rigid linker
IPFTV

SEQ ID NO: 83 - protein, 347 aa, synthetic construct
  REGION 1..347: note = CA497 Fab heavy chain with human IL-15 inserted into framework 3, Gly-Ser linkers
EVQLVESGGGLVKPGGSLRLSCAASGVIFSDYYMAWVRQAPGKGLEWVASINFNADISYYRESVKGRFTISRDDSGGGSGGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINGGGKNTLYLQMNSLKTEDTAVYYCTTDANRQNYDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 84 - protein, 218 aa, synthetic construct
  REGION 1..218: note = CA497 Fab light chain
AIQLTQSPSSLSASVGDRVTITCKASESVSSSMYSYMHWYQQKPGKAPKLLIYRASNLESGVPSRFSGSGSGTDFTLTISSLQPEDFATYYCQQSWTAPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 346 aa, synthetic construct
  REGION 1..346: note = CA240 Fab heavy chain with human IL-15 inserted into framework 3, Gly-Ser linkers
EVQLVESGGGLVQPGGSLRLSCAASGFNFNDYFMNWVRQAPGKGLEWVAQMRNKNYQYGTYYAESLEGRFTISRDDSGGGSGGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINGGGKNSLYLQMNSLKTEDTAVYYCARESYYGFTSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 86 - protein, 214 aa, synthetic construct
  REGION 1..214: note = CA240 Fab light chain
DIQMTQSPSSLSASVGDRVTITCQASQDIGISLSWYQQKPGKAPKLLIYNANNLADGVPSRFSGSGSGTDFTLTISSLQPEDFATYYCLQHNSAPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 219 aa, synthetic construct
  REGION 1..219: note = HyHEL5 heavy chain
EVQLQQSGAELMKPGASVKISCKASGYTFSDYWIEWVKQRPGHGLEWIGEILPGSGSTNYHERFKGKATFTADTSSSTAYMQLNSLTSEDSGVYYCLHGNYDFDGWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 88 - protein, 212 aa, synthetic construct
  REGION 1..212: note = HyHEL5 light chain
DIVLTQSPAIMSASPGEKVTMTCSASSSVNYMYWYQQKSGTSPKRWIYDTSKLASGVPVRFSGSGSGTSYSLTISSMETEDAATYYCQQWGRNPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 127 aa, synthetic construct
  REGION 1..127: note = D3L11
DVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSS

SEQ ID NO: 90 - protein, 360 aa, synthetic construct
  REGION 1..360: note = Anti-lysozyme HyHEL5 light chain with anti-lysozyme VHH D3L11 inserted into framework 3, Gly-Ser linkers
DIVLTQSPAIMSASPGEKVTMTCSASSSVNYMYWYQQKSGTSPKRWIYDTSKLASGVPVRFSGSGSGSGGGGSDVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSTSYSLTISSMETEDAATYYCQQWGRNPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 352 aa, synthetic construct
  REGION 1..352: note = Anti-lysozyme HyHEL5 light chain with anti-IL-6 VHH15 inserted into framework 3, Gly-Ser linkers
DIVLTQSPAIMSASPGEKVTMTCSASSSVNYMYWYQQKSGTSPKRWIYDTSKLASGVPVRFSGSGSGSGGGGSQVQLVESGGGSVQAGGSLRLSCVAASGYTGCTYDMRWYRQAPGKEREFVSGIDSDGRATYADSVKGRFTISQSNAKIAVYLQMDSLKLEDTAMYYCNLQCLRYPGEYYWGQGTQVTVSSGGGGSGGGGSGGGGSTSYSLTISSMETEDAATYYCQQWGRNPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 365 aa, synthetic construct
  REGION 1..365: note = CA645 light chain with anti-lysozyme VHH D3L11 inserted into framework 3, Gly-Ser linkers
DIQMTQSPSSVSASVGDRVTITCQSSPSVWSNFLSWYQQKPGKAPKLLIYEASKLTSGVPSRFSGSGSGSGGGGSDVQLVESGGGSVQAGGSLRLSCAASGSTDSIEYMTWFRQAPGKAREGVAALYTHTGNTYYTDSVKGRFTISQDKAKNMAYLRMDSVKSEDTAIYTCGATRKYVPVRFALDQSSYDYWGQGTQVTVSSGGGGSGGGGSGGGGSTDFTLTISSLQPEDFATYYCGGGYSSISDTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 360 aa, synthetic construct
  REGION 1..360: note = CA645 Fab heavy chain with human IL-15 inserted into framework 3 with bovine long linker
EVQLLESGGGLVQPGGSLRLSCAVSGIDLSNYAINWVRQAPGKGLEWIGIIWASGTTFYATWAKGRFTISRDNSTSVHQETKKYQSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINSYTYNYEWHVDVKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 94 - protein, 350 aa, synthetic construct
  REGION 1..350: note = CA645 Fab heavy chain with human IL-15 inserted into framework 3 with bovine short linker
EVQLLESGGGLVQPGGSLRLSCAVSGIDLSNYAINWVRQAPGKGLEWIGIIWASGTTFYATWAKGRFTISRDNSETKKYQSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINSYTYNYEKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 95 - protein, 356 aa, synthetic construct
  REGION 1..356: note = CA645 Fab heavy chain with human IL-15 inserted into framework 3 with 2xG4S linker
EVQLLESGGGLVQPGGSLRLSCAVSGIDLSNYAINWVRQAPGKGLEWIGIIWASGTTFYATWAKGRFTISRDNSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINGGGGSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 96 - protein, 12 aa, synthetic construct
  REGION 1..12: note = Linker
TSVHQETKKYQS

SEQ ID NO: 97 - protein, 12 aa, synthetic construct
  REGION 1..12: note = Linker
SYTYNYEWHVDV

SEQ ID NO: 98 - protein, 7 aa, synthetic construct
  REGION 1..7: note = Linker
ETKKYQS

SEQ ID NO: 99 - protein, 7 aa, synthetic construct
  REGION 1..7: note = Linker
SYTYNYE

SEQ ID NO: 100 - protein, 188 aa, Homo sapiens
NCWLRQAKNGRCQVLYKTELSKEECCSTGRLSTSWTEEDVNDNTLFKWMIFNGGAPNCIPCKEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAGEYK

SEQ ID NO: 101 - protein, 627 aa, synthetic construct
  REGION 1..627: note = CA645 Fab heavy chain with two human IL-10 monomers inserted into framework 3
EVQLLESGGGLVQPGGSLRLSCAVSGIDLSNYAINWVRQAPGKGLEWIGIIWASGTTFYATWAKGRFTISRDNSGGGGSGSNCWLRQAKNGRCQVLYKTELSKEECCSTGRLSTSWTEEDVNDNTLFKWMIFNGGAPNCIPCKEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAGEYKGGGGSGGGGSGGGGSNCWLRQAKNGRCQVLYKTELSKEECCSTGRLSTSWTEEDVNDNTLFKWMIFNGGAPNCIPCKEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGGGGSGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 102 - protein, 110 aa, synthetic construct
  REGION 1..110: note = CA645-Cys VL domain (gL5)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIK

SEQ ID NO: 103 - protein, 121 aa, synthetic construct
  REGION 1..121: note = CA645-Cys VH domain (gH5)
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 104 - protein, 6 aa, synthetic construct
  REGION 1..6: note = Rigid linker
EYHGLQ

SEQ ID NO: 105 - protein, 119 aa, synthetic construct
  REGION 1..119: note = CA645 VH domain (gH1)
EVQLLESGGGLVQPGGSLRLSCAVSGIDLSNYAINWVRQAPGKGLEWIGIIWASGTTFYATWAKGRFTISRDSTTVYLQMNSLRAEDTAVYYCARTVPGYSTAPYFDLWGQGTLVTVSS

SEQ ID NO: 106 - protein, 121 aa, synthetic construct
  REGION 1..121: note = CA645 VH domain (gH37)
EVQLLESGGGLVQPGGSLRLSCAVSGIDLSNYAINWVRQAPGKGLEWIGIIWASGTTAYATWAKGRFTISRDNSKNTVYLQMNSLRAEDTAVYYCARTVPGYSTAPYFDLWGQGTLVTVSS

SEQ ID NO: 107 - protein, 120 aa, synthetic construct
  REGION 1..120: note = CA645 VH domain (gH47)
EVQLLESGGGLVQPGGSLRLSCAVSGIDLSNYAINWVRQAPGKGLEWIGIIWASGTTFYATWAKGRFTISRDNSKNTVYLQMNSLRAEDAVYYCARTVPGYSAAPYFDLWGQGTLVTVSS

SEQ ID NO: 108 - protein, 110 aa, synthetic construct
  REGION 1..110: note = CA645 VL domain (gL1)
DIVMTQSPSSVSASVGDRVTITCQSSPSVWSNFLSWYQQKPGKAPKLLIYEASKLTSGVPSRFKGSGSGTDFTLTISSLQPEDFATYYCGGGYSSISDTTFGGGTKVEIK

SEQ ID NO: 109 - protein, 110 aa, synthetic construct; identical to SEQ ID NO: 2
  REGION 1..110: note = CA645 VL domain (gL4)

SEQ ID NO: 110 - protein, 112 aa, synthetic construct
  REGION 1..112: note = CA645-Cys VL domain (gL4)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 111 - protein, 11 aa, synthetic construct
  REGION 1..11: note = Albumin binding peptide
DLCLRDWGCLW

SEQ ID NO: 112 - protein, 11 aa, synthetic construct
  REGION 1..11: note = Albumin binding peptide
DICLPRWGCLW

SEQ ID NO: 113 - protein, 15 aa, synthetic construct
  REGION 1..15: note = Albumin binding peptide
MEDICLPRWGCLWGD

SEQ ID NO: 114 - protein, 20 aa, synthetic construct
  REGION 1..20: note = Albumin binding peptide
QRLMEDICLPRWGCLWEDDE

SEQ ID NO: 115 - protein, 20 aa, synthetic construct
  REGION 1..20: note = Albumin binding peptide
QGLIGDICLPRWGCLWGRSV

SEQ ID NO: 116 - protein, 21 aa, synthetic construct
  REGION 1..21: note = Albumin binding peptide
QGLIGDICLPRWGCLWGRSVK

SEQ ID NO: 117 - protein, 15 aa, synthetic construct
  REGION 1..15: note = Albumin binding peptide
EDICLPRWGCLWEDD

SEQ ID NO: 118 - protein, 18 aa, synthetic construct
  REGION 1..18: note = Albumin binding peptide
RLMEDICLPRWGCLWEDD

SEQ ID NO: 119 - protein, 16 aa, synthetic construct
  REGION 1..16: note = Albumin binding peptide
MEDICLPRWGCLWEDD

SEQ ID NO: 120 - protein, 15 aa, synthetic construct
  REGION 1..15: note = Albumin binding peptide
MEDICLPRWGCLWED

SEQ ID NO: 121 - protein, 18 aa, synthetic construct
  REGION 1..18: note = Albumin binding peptide
RLMEDICLARWGCLWEDD

SEQ ID NO: 122 - protein, 20 aa, synthetic construct
  REGION 1..20: note = Albumin binding peptide
EVRSFCTRWPAEKSCKPLRG

SEQ ID NO: 123 - protein, 20 aa, synthetic construct
  REGION 1..20: note = Albumin binding peptide
RAPESFVCYWETICFERSEQ

SEQ ID NO: 124 - protein, 11 aa, synthetic construct
  REGION 1..11: note = Albumin binding peptide
EMCYFPGICWM

SEQ ID NO: 125 - protein, 224 aa, synthetic construct
  REGION 1..224: note = CA497 Fab Heavy Chain
EVQLVESGGGLVKPGGSLRLSCAASGVIFSDYYMAWVRQAPGKGLEWVASINFNADISYYRESVKGRFTISRDDSKNTLYLQMNSLKTEDTAVYYCTTDANRQNYDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 126 - protein, 223 aa, synthetic construct
  REGION 1..223: note = CA240 Fab Heavy chain
EVQLVESGGGLVQPGGSLRLSCAASGFNFNDYFMNWVRQAPGKGLEWVAQMRNKNYQYGTYYAESLEGRFTISRDDSKNSLYLQMNSLKTEDTAVYYCARESYYGFTSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 127 - protein, 247 aa, synthetic construct
  REGION 1..247: note = CA497 dsscFv (vHvL)
EVQLVESGGGLVKPGGSLRLSCAASGVIFSDYYMAWVRQAPGKCLEWVASINFNADISYYRESVKGRFTISRDDSKNTLYLQMNSLKTEDTAVYYCTTDANRQNYDWFAYWGQGTLVTVSSGGGGSGGGGSGGGGSAIQLTQSPSSLSASVGDRVTITCKASESVSSSMYSYMHWYQQKPGKAPKLLIYRASNLESGVPSRFSGSGSGTDFTLTISSLQPEDFATYYCQQSWTAPRTFGCGTKVEIK

SEQ ID NO: 128 - protein, 247 aa, synthetic construct
  REGION 1..247: note = CA497 dsscFv (vLvH)
AIQLTQSPSSLSASVGDRVTITCKASESVSSSMYSYMHWYQQKPGKAPKLLIYRASNLESGVPSRFSGSGSGTDFTLTISSLQPEDFATYYCQQSWTAPRTFGCGTKVEIKGGGGSGGGGSGGGGSEVQLVESGGGLVKPGGSLRLSCAASGVIFSDYYMAWVRQAPGKCLEWVASINFNADISYYRESVKGRFTISRDDSKNTLYLQMNSLKTEDTAVYYCTTDANRQNYDWFAYWGQGTLVTVSS

SEQ ID NO: 129 - protein, 491 aa, synthetic construct
  REGION 1..491: note = CA645 Fab HC Fwk3 CA497 dsscFv (vHvL)
EVQLLESGGGLVQPGGSLRLSCAVSGIDLSNYAINWVRQAPGKGLEWIGIIWASGTTFYATWAKGRFTISRDNSGGGGSGGGGSEVQLVESGGGLVKPGGSLRLSCAASGVIFSDYYMAWVRQAPGKCLEWVASINFNADISYYRESVKGRFTISRDDSKNTLYLQMNSLKTEDTAVYYCTTDANRQNYDWFAYWGQGTLVTVSSGGGGSGGGGSGGGGSAIQLTQSPSSLSASVGDRVTITCKASESVSSSMYSYMHWYQQKPGKAPKLLIYRASNLESGVPSRFSGSGSGTDFTLTISSLQPEDFATYYCQQSWTAPRTFGCGTKVEIKGGGGSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 130 - protein, 491 aa, synthetic construct
  REGION 1..491: note = CA645 Fab HC Fwk3 CA497 dsscFv (vLvH)
EVQLLESGGGLVQPGGSLRLSCAVSGIDLSNYAINWVRQAPGKGLEWIGIIWASGTTFYATWAKGRFTISRDNSGGGGSGGGGSAIQLTQSPSSLSASVGDRVTITCKASESVSSSMYSYMHWYQQKPGKAPKLLIYRASNLESGVPSRFSGSGSGTDFTLTISSLQPEDFATYYCQQSWTAPRTFGCGTKVEIKGGGGSGGGGSGGGGSEVQLVESGGGLVKPGGSLRLSCAASGVIFSDYYMAWVRQAPGKCLEWVASINFNADISYYRESVKGRFTISRDDSKNTLYLQMNSLKTEDTAVYYCTTDANRQNYDWFAYWGQGTLVTVSSGGGGSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 131 - protein, 718 aa, synthetic construct
  REGION 1..718: note = CA645 IgG4P HC Fwk3 CA497 dsscFv (vHvL)
EVQLLESGGGLVQPGGSLRLSCAVSGIDLSNYAINWVRQAPGKGLEWIGIIWASGTTFYATWAKGRFTISRDNSGGGGSGGGGSEVQLVESGGGLVKPGGSLRLSCAASGVIFSDYYMAWVRQAPGKCLEWVASINFNADISYYRESVKGRFTISRDDSKNTLYLQMNSLKTEDTAVYYCTTDANRQNYDWFAYWGQGTLVTVSSGGGGSGGGGSGGGGSAIQLTQSPSSLSASVGDRVTITCKASESVSSSMYSYMHWYQQKPGKAPKLLIYRASNLESGVPSRFSGSGSGTDFTLTISSLQPEDFATYYCQQSWTAPRTFGCGTKVEIKGGGGSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 718 aa, synthetic construct
  REGION 1..718: note = CA645 IgG4P HC Fwk3 CA497 dsscFv (vLvH)
EVQLLESGGGLVQPGGSLRLSCAVSGIDLSNYAINWVRQAPGKGLEWIGIIWASGTTFYATWAKGRFTISRDNSGGGGSGGGGSAIQLTQSPSSLSASVGDRVTITCKASESVSSSMYSYMHWYQQKPGKAPKLLIYRASNLESGVPSRFSGSGSGTDFTLTISSLQPEDFATYYCQQSWTAPRTFGCGTKVEIKGGGGSGGGGSGGGGSEVQLVESGGGLVKPGGSLRLSCAASGVIFSDYYMAWVRQAPGKCLEWVASINFNADISYYRESVKGRFTISRDDSKNTLYLQMNSLKTEDTAVYYCTTDANRQNYDWFAYWGQGTLVTVSSGGGGSGGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 213 aa, synthetic construct
  REGION 1..213: note = Anti-CD3 Light chain
DIQMTQSPSSLSASVGDRVTITCSASSSVSYMNWYQQTPGKAPKLLIYDTSKLASGVPSRFSGSGSGTDYTFTISSLQPEDIATYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 222 aa, synthetic construct
  REGION 1..222: note = Anti-CD3 Fab HC
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 135 - protein, 446 aa, synthetic construct
  REGION 1..446: note = Anti-CD3 IgG4P HC
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 214 aa, synthetic construct
  REGION 1..214: note = Anti-CD28 Light Chain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 223 aa, synthetic construct
  REGION 1..223: note = Anti-CD28 Fab HC
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 138 - protein, 447 aa, synthetic construct
  REGION 1..447: note = Anti-CD28 IgG4P HC
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - protein, 488 aa, synthetic construct
  REGION 1..488: note = Anti-CD28 Fab HC Fwk3 anti-CD3 dsscFv (vHvL)
QVQLVQSGAEVKKPGASVKVSCKASGYTFTSYYIHWVRQAPGQGLEWIGCIYPGNVNTNYNEKFKDRATLTVDTSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYCLDYWGQGTPVTVSSGGGGSGGGGSGGGGSGGGGSDIQMTQSPSSLSASVGDRVTITCSASSSVSYMNWYQQTPGKAPKLLIYDTSKLASGVPSRFSGSGSGTDYTFTISSLQPEDIATYYCQQWSSNPFTFGCGTKLEIKGGGGSGGGGSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 140 - protein, 487 aa, synthetic construct
  REGION 1..487: note = Anti-CD3 Fab HC Fwk3 anti-CD3 dsscFv (vHvL)
QVQLVQSGGGVVQPGRSLRLSCKASGYTFTRYTMHWVRQAPGKGLEWIGYINPSRGYTNYNQKFKDRFTISRDNSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYCLDYWGQGTPVTVSSGGGGSGGGGSGGGGSGGGGSDIQMTQSPSSLSASVGDRVTITCSASSSVSYMNWYQQTPGKAPKLLIYDTSKLASGVPSRFSGSGSGTDYTFTISSLQPEDIATYYCQQWSSNPFTFGCGTKLEIKGGGGSGGGGSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 141 - protein, 712 aa, synthetic construct
  REGION 1..712: note = Anti-CD28 IgG4P HC Fwk3 anti-CD3 dsscFv (vHvL)
QVQLVQSGAEVKKPGASVKVSCKASGYTFTSYYIHWVRQAPGQGLEWIGCIYPGNVNTNYNEKFKDRATLTVDTSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYCLDYWGQGTPVTVSSGGGGSGGGGSGGGGSGGGGSDIQMTQSPSSLSASVGDRVTITCSASSSVSYMNWYQQTPGKAPKLLIYDTSKLASGVPSRFSGSGSGTDYTFTISSLQPEDIATYYCQQWSSNPFTFGCGTKLEIKGGGGSGGGGSISTAYMELSRLRSDDTAVYFCTRSHYGLDWNFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 142 - protein, 711 aa, synthetic construct
  REGION 1..711: note = Anti-CD3 IgG4P HC Fwk3 anti-CD3 dsscFv (vHvL)
QVQLVQSGGGVVQPGRSLRLSCKASGYTFTRYTMHWVRQAPGKGLEWIGYINPSRGYTNYNQKFKDRFTISRDNSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYCLDYWGQGTPVTVSSGGGGSGGGGSGGGGSGGGGSDIQMTQSPSSLSASVGDRVTITCSASSSVSYMNWYQQTPGKAPKLLIYDTSKLASGVPSRFSGSGSGTDYTFTISSLQPEDIATYYCQQWSSNPFTFGCGTKLEIKGGGGSGGGGSKNTAFLQMDSLRPEDTGVYFCARYYDDHYCLDYWGQGTPVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 143 - protein, 251 aa, synthetic construct
  REGION 1..251: note = CA645 scFv (VH-VL)
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGQGTKVEIK

SEQ ID NO: 144 - protein, 251 aa, synthetic construct
  REGION 1..251: note = CA645 dsscFv (VH-VL)
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIK